SEQUENCE LISTING (38 sequences)

SEQ ID NO: 1 - protein, 124 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNEN

SEQ ID NO: 2 - protein, 369 aa, Artificial Sequence
  Fusion polypeptide - Human CD47/FC mutein
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENSKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 277 aa, variola minor virus
MLRVRILLIYLCTFVVITSTKTIEYTACNDTIIIPCTIDNPTKYIRWKLDNHNILTYNKTSKTIILSKWHTSAKLHSLSDNDVSLIIKYKDILPGTYTCEDNTGIKSTVKLVQRHTNWFNDHHTMLMFIFTGITLFLLFLEIAYTSISVVFSTNLGILQVFGCIIAMIELCGAFLFYPSMFTLRHIIGLLMMTLPSIFLIITKVFSFWLLCKLSCAVHLIIYYQLAGYILTVLGLGLSLKECVDGTLLLSGLGTIMVSEHFSLLFLVCFPSTQRDYY

SEQ ID NO: 4 - protein, 105 aa, variola minor
KTIEYTACNDTIIIPCTIDNPTKYIRWKLDNHNILTYNKTSKTIILSKWHTSAKLHSLSDNDVSLIIKYKDILPGTYTCEDNTGIKSTVKLVQRHTNWFNDHHTM

SEQ ID NO: 5 - protein, 351 aa, Artificial Sequence
  Human CD47/FC mutein fusion polypeptide
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENSKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 228 aa, Homo sapiens
CDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 226 aa, Artificial Sequence
  Mutated human sequence
KTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 228 aa, Artificial Sequence
  Mutated human sequence
CDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 10 - protein, 305 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 11 - protein, 142 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNEN

SEQ ID NO: 12 - protein, 124 aa, Artificial Sequence
  Mutated human sequence
QLLFNKTKSVEFTFSNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNEN

SEQ ID NO: 13 - DNA, 5313 bp, Homo sapiens
ggggagcaggcgggggagcgggcgggaagcagtgggagcgcgcgtgcgcgcggccgtgcagcctgggcagtgggtcctgcctgtgacgcgcggcggcggtcggtcctgcctgtaacggcggcggcggctgctgctccagacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtc

SEQ ID NO: 14 - DNA, 5346 bp, Homo sapiens
ggggagcaggcgggggagcgggcgggaagcagtgggagcgcgcgtgcgcgcggccgtgcagcctgggcagtgggtcctgcctgtgacgcgcggcggcggtcggtcctgcctgtaacggcggcggcggctgctgctccagacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtc

SEQ ID NO: 15 - protein, 305 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 16 - DNA, 5288 bp, Homo sapiens
ggggagcaggcgggggagcgggcgggaagcagtgggagcgcgcgtgcgcgcggccgtgcagcctgggcagtgggtcctgcctgtgacgcgcggcggcggtcggtcctgcctgtaacggcggcggcggctgctgctccagacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtc

SEQ ID NO: 17 - protein, 312 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNE

SEQ ID NO: 18 - protein, 18 aa, Artificial Sequence
  Signal peptide of human CD47
MWPLVAALLLGSACCGSA

SEQ ID NO: 19 - DNA, 5346 bp, Homo sapiens; identical to SEQ ID NO: 14

SEQ ID NO: 20 - DNA, 1273 bp, Homo sapiens
tgcctgtgacgcgcggcggcggtcggtcctgcctgtaacggcggcggcggctgctgctccagacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaaaaaaaaaaaaaaaaaaaaaaaaaaaaaaaaaaa

SEQ ID NO: 21 - protein, 6 aa, Artificial Sequence
  Residues in the amino terminal portion of the CH2 domain that contribute to IgG Fc receptor binding
LLGGPS

SEQ ID NO: 22 - protein, 18 aa, Artificial Sequence
  Amino acid sequence at amino terminal end of an exemplary mutein Fc polypeptide.
KTHTCPPCPAPEAEGAPS

SEQ ID NO: 23 - protein, 227 aa, Artificial Sequence
  Mutated human mutein FC
SKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 8 aa, Artificial Sequence
  FLAG(r) epitope tag
DYKDDDDK

SEQ ID NO: 25 - protein, 8 aa, Artificial Sequence
  XPRESS (TM) epitope tag
DLYDDDDK

SEQ ID NO: 26 - protein, 20 aa, variola virus
MLRVRILLIYLCTFVVITST

SEQ ID NO: 27 - protein, 305 aa, Homo sapiens; identical to SEQ ID NO: 15

SEQ ID NO: 28 - protein, 608 aa, Artificial Sequence
  Human fusion polypeptide
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENKTHTCPPCPAPELLGGPSVFLFPPKPKDTLMISRTPEVTCVVVDVSHEDPEVKFNWYVDGVEVHNAKTKPREEQYSTYRVVSVLTVLHQDWLNGKEYKMWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENKTHTCPPCPAPELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 367 aa, Artificial Sequence
  Human fusion polypeptide
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 5 aa, Artificial Sequence
  Gly-Ser linker sequence
GGGGS

SEQ ID NO: 31 - protein, 11 aa, Artificial Sequence
  Gly-Ser linker sequence
GGGGSGGGGGS

SEQ ID NO: 32 - protein, 372 aa, Artificial Sequence
  Human fusion polypeptide
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 377 aa, Artificial Sequence
  Human fusion polypeptide
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 106 aa, Artificial Sequence
  Peptide sequence of combined affinity tags
AGGPGGYPYDVPDYAEDQVDPRLIDGKMDEKTTGWRGGHVVEGLAGELEQLRARLEHHPQGQREPGSGMDEKTTGWRGGHVVEGLAGELEQLRARLEHHPQGQREP

SEQ ID NO: 35 - DNA, 327 bp, Artificial Sequence
  Nucleotide sequence of combined affinity tags
gcggccgctggcggccccggcggctacccctacgacgtgcccgactacgccgaggaccaggtggacccccggctgatcgacggcaagatggacgagaagaccaccggctggcggggcggccacgtggtggagggcctggccggcgagctggagcagctgcgggcccggctggagcaccacccccagggccagcgggagcccggaagcggtatggatgaaaaaactactggttggagagggggacatgtagtcgaaggtctggccggcgagttagaacaattaagagctagattggaacatcatccacaaggtcaaagagaaccttag

SEQ ID NO: 36 - DNA, 1928 bp, Mus musculus
cccgggcagcctgggcggccgctcctgcctgtcactgctgcggcgctgctggtcggtcgtttcccttgaaggcagcagcggaggcggcggctgctccagacacctgcggcggcgaccccccggcggcgcggagatgtggcccttggcggcggcgctgttgctgggctcctgctgctgcggttcagctcaactactgtttagtaacgtcaactccatagagttcacttcatgcaatgaaactgtggtcatcccttgcatcgtccgtaatgtggaggcgcaaagcaccgaagaaatgtttgtgaagtggaagttgaacaaatcgtatattttcatctatgatggaaataaaaatagcactactacagatcaaaactttaccagtgcaaaaatctcagtctcagacttaatcaatggcattgcctctttgaaaatggataagcgcgatgccatggtgggaaactacacttgcgaagtgacagagttatccagagaaggcaaaacagttatagagctgaaaaaccgcacggccttcaacactgaccaaggatcagcctgttcttacgaggaggagaaaggaggttgcaaattagtttcgtggttttctccaaatgaaaagatcctcattgttattttcccaattttggctatactcctgttctggggaaagtttggtattttaacactcaaatataaatccagccatacgaataagagaatcattctgctgctcgttgccgggctggtgctcacagtcatcgtggttgttggagccatccttctcatcccaggagaaaagcccgtgaagaatgcttctggacttggcctcattgtaatctctacggggatattaatactacttcagtacaatgtgtttatgacagcttttggaatgacctctttcaccattgccatattgatcactcaagtgctgggctacgtccttgctttggtcgggctgtgtctctgcatcatggcatgtgagccagtgcacggcccccttttgatttcaggtttggggatcatagctctagcagaactacttggattagtttatatgaagtttgtcgcttccaaccagaggactatccaacctcctaggaataggtgaagggaagtgacggactgtaacttggaagtcagaaatggaagaatacagttgtctaagcaccaggtcttcacgactcacagctggaaggaacagacaacagtaactgacttccatccaggaaaacatgtcacataaatgattactaagtttatattcaaagcagctgtactttacataataaaaaaaatatgatgtgctgtgtaaccaattggaatcccatttttctattgtttctactcaactaggggcaaacgtttcaggggcaacttccaagaatgatgcttgttagatcctagagtctctgaacactgagtttaaattgattccgagtgagactcgccaagcactaacctgagggttagttacccagagatacctatgaaaaacagtggtatccagcaagccttagtaaactcaggttgccagcagctttgccacttccgctgctagctgaataacaagactgccacttctgggtcatagtgatagagactgaagtagaaaaacgaatgtggttgggcaaatcccgtgtggcccctctgtgtgctatgatattgatggcactggtgtcttcattcttgggggttgccatcattcacacacacccctttgacatacagtgcaccccagttttgaatacattttttttgcaccctgtcccgttctgctactttgatttgcgttatgatatatatatatatatataataccttttctcctctttaaacatggtcctgtgacacaatagtcagttgcagaaaggagccagacttattcgcaaagcactgtgctcaaactcttcagaaaaaaaaaaaaaaaa

SEQ ID NO: 37 - protein, 324 aa, Mus musculus
MWPLAAALLLGSCCCGSAQLLFSNVNSIEFTSCNETVVIPCIVRNVEAQSTEEMFVKWKLNKSYIFIYDGNKNSTTTDQNFTSAKISVSDLINGIASLKMDKRDAMVGNYTCEVTELSREGKTVIELKNRTAFNTDQGSACSYEEEKGGCKLVSWFSPNEKILIVIFPILAILLFWGKFGILTLKYKSSHTNKRIILLLVAGLVLTVIVVVGAILLIPGEKPVKNASGLGLIVISTGILILLQYNVFMTAFGMTSFTIAILITQVLGYVLALVGLCLCIMACEPVHGPLLISGLGIIALAELLGLVYMKFVASNQRTIQPPRNR

SEQ ID NO: 38 - protein, 303 aa, Mus musculus
MWPLAAALLLGSCCCGSAQLLFSNVNSIEFTSCNETVVIPCIVRNVEAQSTEEMFVKWKLNKSYIFIYDGNKNSTTTDQNFTSAKISVSDLINGIASLKMDKRDAMVGNYTCEVTELSREGKTVIELKNRTVSWFSPNEKILIVIFPILAILLFWGKFGILTLKYKSSHTNKRIILLLVAGLVLTVIVVVGAILLIPGEKPVKNASGLGLIVISTGILILLQYNVFMTAFGMTSFTIAILITQVLGYVLALVGLCLCIMACEPVHGPLLISGLGIIALAELLGLVYMKFVASNQRTIQPPRNR

(19) United States
(12) Patent Application Publication
(10) Pub. No.: US 2008/0131431 A1
(43) Pub. Date: Jun. 5, 2008

(54) CD47 related compositions and methods for treating immunological diseases and disorders

(75) Inventors: Craig A. Smith, Seattle, WA (US); Steven Wiley, Seattle, WA (US); Ajamete Kaykas, Seattle, WA (US); Peter Probst, Seattle, WA (US)

Correspondence Address: SEED INTELLECTUAL PROPERTY LAW GROUP PLLC, 701 FIFTH AVE, SUITE 5400, SEATTLE, WA, 98104, US

(73) Assignee: Viral Logic Systems Technology Corp., Seattle, WA (US)

(21) Appl. No.: 11/804,992

(22) Filed: May 15, 2007

Related U.S. Application Data
(60) Provisional application No. 60/800,643, filed on May 15, 2006.

(51) Int. Cl. A61K 39/00; C07K 16/00; C07H 21/04; C12N 5/06; G01N 33/53; C12N 5/10; C12N 15/00; C12N 5/00

(52) U.S. Cl. 424/134.1; 530/387.3; 536/23.4; 435/320.1; 435/325; 435/375; 530/387.1; 435/346; 424/130.1; 435/7.21

(57) ABSTRACT

Provide herein are fusion polypeptides that comprise a CD47 extracellular domain or a variant thereof that is fused to a Fc polypeptide. The fusion polypeptides are useful for treating an immunological disease or disorder in a subject according to the methods described herein. The fusion polypeptides are capable of suppressing immunoresponsiveness of an immune cell, inhibiting production of proinflammatory cytokines, including inhibiting immune complex-induced production of cytokines.

### CROSS-REFERENCE TO RELATED APPLICATION

This application claims the benefit of U.S. Provisional Application No. 60/800,643 filed May 15, 2006, which is incorporated herein by reference in its entirety.

### STATEMENT REGARDING SEQUENCE LISTING SUBMITTED ON CD-ROM

The Sequence Listing associated with this application is provided on CD-ROM in lieu of a paper copy, and is hereby incorporated by reference into the specification. Three CD-ROMs are provided, containing identical copies of the sequence listing: CD-ROM No. 1 is labeled COPY 1, contains the file 405.app.txt which is 86 KB and created on May 15, 2007; CD-ROM No. 2 is labeled COPY 2, contains the file 405.app.txt which is 86 KB and created on May 15, 2007; CD-ROM No. 3 is labeled CRF (Computer Readable Form), contains the file 405.app.txt which is 86 KB and created on May 15, 2007.

### BACKGROUND

1. Field

Provided herein are CD47 fusion polypeptides and related compositions that may be useful for treating immunological diseases and disorders, including autoimmune diseases and disorders. The fusion polypeptides described herein alter immunoresponsiveness of the immune cell, such as by inhibiting production of cytokines by the immune cell.

2. Description of the Related Art

Viruses, such as members of poxvirus families, have the capability to evolve and/or the capability to acquire genes from the host that modulate an immune response of the host to the virus and/or that facilitate viral replication (Bugert and Darai, Virus Genes 21: 111 (2000); Alcami et al., Semin. Virol. 8:419 (1998); McFadden and Barry, Semin. Virol. 8:429 (1998)). A cellular component in the host that is a ligand for a viral virulence factor may, therefore, be an important immunomodulatory target. Poxviruses form a group of double-stranded DNA viruses that replicate in the cytoplasm of a cell and that have adapted to replicate in numerous different hosts. Poxviruses, including variola virus, the causative agent of smallpox, and vaccinia virus, a prototype poxvirus widely used as a smallpox vaccine, have large genomes of nearly 190 kilobases, which could potentially encode more than 200 proteins (see, e.g., Goebel et al., Virology 179:247 (1990)).

Identification of poxvirus virulence factors that are capable of suppressing the host's immune system is useful for identifying cellular polypeptides that are effectors or modulators of an immune response and that can be modulated in a manner that is beneficial for treating immunological disorders, such as, for example, inflammatory diseases and autoimmune diseases, including multiple sclerosis, rheumatoid arthritis, and systemic lupus erythematosus (SLE).

Immunological diseases and disorders afflict more than twenty million people in the United States. Many immunological diseases are debilitating and chronic and thus affect a patient's productivity, well-being, as well as general health. A need exists to identify and develop compositions that can be used for treatment and prophylaxis of such immunological diseases and disorders.

### BRIEF SUMMARY

In one embodiment, a fusion polypeptide is provided wherein the fusion polypeptide comprises an extracellular domain of CD47 fused to a human IgG1 Fc polypeptide, wherein the Fc polypeptide comprises a substitution or a deletion of a cysteine residue in the hinge region, wherein the substituted or deleted cysteine residue is the cysteine residue most proximal to the amino terminus of the hinge region of the Fc portion of a wildtype human IgG1 immunoglobulin, and wherein the Fc polypeptide further comprises a substitution or deletion of an aspartate residue immediately adjacent to the cysteine residue most proximal to the amino terminus of the hinge region of the Fc portion of a wildtype human IgG1 immunoglobulin, wherein the fusion polypeptide alters the immunoresponsiveness of an immune cell. In another embodiment, the fusion polypeptide comprises an amino acid sequence at least 85% identical to the amino acid sequence set forth in SEQ ID NO:2. In another specific embodiment, the fusion polypeptide comprises an amino acid sequence at least 90% identical to the amino acid sequence set forth in SEQ ID NO:2. In yet another embodiment, the fusion polypeptide comprises an amino acid sequence at least 95% identical to the amino acid sequence set forth in SEQ ID NO:2. In a particular embodiment, the extracellular domain of human CD47 comprises an amino acid sequence at least 95% identical to the amino acid sequence set forth in SEQ ID NO:1 wherein the cysteine residues located at positions corresponding to positions 15, 23, and 96 of SEQ ID NO:1 are retained. In another embodiment, the extracellular domain of human CD47 comprises the amino acid sequence set forth in SEQ ID NO:1. In yet another embodiment, the extracellular domain of human CD47 comprises an amino acid sequence at least 95% identical to the sequence set forth in SEQ ID NO:11 wherein the cysteine residues located at positions corresponding to positions 33, 41, and 114 of SEQ ID NO:11 are retained. In a more specific embodiment, the extracellular domain of human CD47 comprises the amino acid sequence set forth in SEQ ID NO:11.

In certain embodiments, the fusion polypeptide described above and herein is capable of inhibiting immune complex-induced cytokine production in the immune cell. In a particular embodiment, production of the cytokine IL-23 is inhibited. In yet another specific embodiment, production of at least one of cytokine selected from IL-23, IL-12, IL-6, and TNF-α is inhibited.

In other specific embodiments, the fusion polypeptide, the Fc polypeptide moiety of the fusion polypeptide comprises at least one amino acid substitution to remove a glycosylation site. In a particular embodiment, the Fc polypeptide moiety is aglycosylated. In other specific embodiments, the fusion polypeptide further comprises a polypeptide spacer between the Fc polypeptide and the extracellular domain of CD47. In one certain embodiment, the polypeptide spacer comprises from 5 to 100 amino acid residues independently selected from glycine, asparagine, serine, threonine, and alanine. In another specific embodiment, the polypeptide spacer comprises 5 to 20 amino acid residues. In a particular embodiment, the polypeptide spacer comprises (Gly₄Ser)ₙ wherein n=1-12.

In yet other specific embodiments, the human IgG1 Fc polypeptide moiety of the fusion polypeptide as described above and herein further comprises substitution of (a) at least one amino acid in the CH2 domain of the Fc polypeptide; (b) at least two amino acid residues in the CH2 domain of the Fc polypeptide or (c) at least three amino acid residues in the CH2 domain of the Fc polypeptide, such that the capability of the fusion polypeptide to bind to an IgG Fc receptor is reduced.

In other particular embodiment, the fusion polypeptide described above and herein forms a dimer of two fusion polypeptide monomers, wherein the dimer comprises a disulfide bond between each of the extracellular CD47 domain moieties of each of the two fusion polypeptide monomers. In a certain embodiment, the disulfide bond between each of the extracellular domain moieties is formed between a cysteine residue of each extracellular CD47 domain moiety, which cysteine residue of each extracellular CD47 domain moiety is most proximal to the amino terminus.

In particular embodiments, the CD47 extracellular domain moiety, and a variant of the CD47 extracellular domain, retains the capability to bind at least one CD47 ligand selected from SIRP-α, SIRP-beta-2, thrombospondin-1, αᵥβ₃ integrin, and α₂β₁ integrin. In another embodiment, the fusion polypeptide (a) competitively inhibits binding of at least one CD47 ligand to a CD47 polypeptide expressed on the cell surface of a cell and (b) competitively inhibits binding of a viral CD47-like polypeptide to at least one CD47 ligand. In certain embodiments, the at least one CD47 ligand is selected from SIRP-α, SIRP-beta-2, thrombospondin-1, αᵥβ₃ integrin, and α₂β₁ integrin. In specific embodiments, the fusion polypeptide competitively inhibits binding of at least one CD47 ligand to a cellular CD47 polypeptide by binding to the at least one CD47 ligand. Also as described above and herein, the fusion polypeptide the fusion polypeptide alters the immunoresponsiveness of an immune cell wherein altering immunoresponsiveness of the immune cell comprises at least one of (a) altering cell migration; (b) inhibiting production of at least one cytokine selected from TNF-α, IL-12, IL-23, IFN-γ, GM-CSF, and IL-6; (c) inhibiting maturation of a dendritic cell; (d) impairing development of a naïve T cell into a Th1 effector cell; (e) inhibiting activation of the immune cell wherein the immune cell expresses SIRP-α on the cell surface; (f) inhibiting production of a chemokine; and (g) suppressing a proinflammatory response by the immune cell. In certain specific embodiments, the immune cell expresses SIRP-α on the cell surface, and altering immunoresponsiveness of the immune cell comprises at least one of (a) inhibiting production of at least one cytokine in the immune cell wherein the cytokine is selected from TNF-α, IL-12, and IL-23; (b) inhibiting immune complex-induced cytokine production in the immune cell; and (c) inhibiting production of at least one chemokine, wherein the chemokine is MIP-1α. In another specific embodiment, the immune cell is a dendritic cell.

In certain embodiments, the fusion polypeptide is a recombinant fusion polypeptide, wherein the recombinant fusion polypeptide comprises the extracellular domain of CD47 fused in frame with the Fc polypeptide.

Also provided herein is a polynucleotide encoding any of the fusion polypeptides described above and herein. Also provided herein is a recombinant expression construct comprising the polynucleotide that is operatively linked to an expression control sequence. In another embodiment, is provided a host cell that is transformed or transfected with the recombinant expression construct.

In another embodiment, a composition is provided wherein the composition comprises any of the aforementioned CD47-Fc fusion polypeptides or any fusion polypeptide described herein and a pharmaceutically suitable carrier. In yet another embodiment, a method of altering an immune response in a subject is provided wherein the method comprises administering to the subject the composition, thereby altering the immune response in the subject. In a particular embodiment, altering the immune response comprises suppressing the immune response.

In yet another embodiment, a method of activating an immune cell is provided wherein the method comprises contacting the immune cell with any one of the aforementioned fusion polypeptides or any CD47 extracellular domain fusion polypeptide described herein, under conditions and a time sufficient to permit the immune cell and the fusion polypeptide to interact, wherein a CD47 ligand is present on the cell surface of the immune cell, thereby activating the immune cell. In particular embodiments, the CD47 ligand is at least one of SIRP-α, SIRP-beta-2, thrombospondin-1, αᵥβ₃ integrin, and α₂β₁ integrin. In a particular embodiment, the CD47 ligand is SIRP-α. In yet another embodiment, activating the immune cell comprises inhibiting Fc-mediated cytokine production. In a more specific embodiment, inhibiting Fc-mediated chemokine production comprises inhibiting immune complex-induced cytokine production. In another specific embodiment, activating the immune cell comprises inhibiting Fc-mediated chemokine production. In yet another particular embodiment, the immune cell is a dendritic cell, a monocyte, a granulocyte, or a bone marrow stem cell.

In one embodiment, a method of inhibiting immune complex-induced cytokine production in an immune cell is provided wherein the method comprises contacting an immune cell with any of the above-mentioned fusion polypeptides or any CD47 extracellular domain fusion polypeptide described herein, under conditions and for a time sufficient to inhibit immune complex-induced cytokine production in the immune cell. In still another embodiment, the immune cell expresses a CD47 ligand on the cell surface.

In particular embodiments, the CD47 ligand is selected from SIRP-α, SIRP-beta-2, thrombospondin-1, αᵥβ₃ integrin, and α₂β₁ integrin. In another specific embodiment, the immune cell is a dendritic cell, a monocyte, a granulocyte, or a bone marrow stem cell. In a more specific embodiment, the immune cell is a dendritic cell and the CD47 ligand is SIRP-α. In a particular embodiment, production of at least one of cytokine selected from IL-23, IL-12, IL-6, and TNF-α is inhibited. In another particular embodiment, the fusion polypeptide inhibits binding of the immune complex to the immune cell.

In one embodiment, a method is provided for treating an immunological disease or disorder in a subject who has or who is at risk of developing the immunological disease or disorder, wherein the method comprises administering to the subject the composition any one of the aforementioned fusion polypeptides, or any CD47 extracellular domain fusion polypeptide described herein, and a pharmaceutically suitable carrier. In a particular embodiment, the immunological disease or disorder is an autoimmune disease or an inflammatory disease. In another particular embodiment, the autoimmune or inflammatory disease is multiple sclerosis, rheumatoid arthritis, a spondyloarthropathy, systemic lupus erythematosus, an antibody-mediated inflammatory or autoimmune disease, graft versus host disease, sepsis, diabetes, psoriasis, atherosclerosis, Sjogren's syndrome, progressive systemic sclerosis, scleroderma, acute coronary syndrome, ischemic reperfusion, Crohn's Disease, endometriosis, glomerulonephritis, myasthenia gravis, idiopathic pulmonary fibrosis, asthma, acute respiratory distress syndrome (ARDS), vasculitis, or inflammatory autoimmune myositis. In yet another specific embodiment, the spondyloarthropathy is selected from ankylosing spondylitis, reactive arthritis, enteropathic arthritis associated with inflammatory bowel disease, psoriatic arthritis, isolated acute anterior uveitis, undifferentiated spondyloarthropathy, Behcet's syndrome, and juvenile idiopathic arthritis. In one embodiment, the immunological disease or disorder is caused by or exacerbated by binding of an immune complex to an immune cell. In a certain particular embodiment, the immune cell is a dendritic cell.

In another embodiment, a method of manufacture is provided for producing any one of the aforementioned fusion polypeptides or any CD47 extracellular domain fusion polypeptide described herein.

Also provided in another embodiment, is an isolated antibody, or antigen-binding fragment thereof, (a) that specifically binds to CD47 and (b) that competitively inhibits binding of a CD47 ligand (i) to CD47 and (ii) to a viral CD47-like polypeptide. In one embodiment, the viral CD47-like polypeptide is a poxvirus CD47-like polypeptide. In a specific embodiment, the poxvirus CD47-like polypeptide is a variola virus CD47-like polypeptide comprising the amino acid sequence set forth in SEQ ID NO:3. In another particular embodiment, the CD47 ligand is selected from SIRP-α, SIRP-beta-2, thrombospondin-1, αᵥβ₃ integrin, and α₂β₁ integrin. In a specific embodiment, the antibody, or antigen-binding fragment thereof, inhibits Fc-mediated cytokine production or chemokine production by an immune cell. In a particular embodiment, inhibiting Fc-mediated cytokine production or chemokine production comprises inhibiting immune complex-induced cytokine production or chemokine production by an immune cell. In specific embodiments, the immune cell is a dendritic cell and the cytokine is selected from IL-12, IL-6, IL-23, and TNF-α. In other specific embodiments, the chemokine is MIP-1α. In another specific embodiment, the antibody is a monoclonal antibody or a polyclonal antibody. In certain embodiments, the monoclonal antibody is selected from a mouse monoclonal antibody, a human monoclonal antibody, a rat monoclonal antibody, and a hamster monoclonal antibody. In other certain embodiments, the antibody is a humanized antibody or a chimeric antibody. In another embodiment, an isolated antibody is provided that comprises an anti-idiotype antibody, or antigen-binding fragment thereof, that specifically binds to the aforementioned antibody, or antigen-binding fragment thereof. In certain specific embodiments, the anti-idiotype antibody is a polyclonal antibody or a monoclonal antibody. Also provided is a host cell that expresses any of the aforementioned antibodies or antigen-binding fragments thereof, including the anti-idiotype antibody. In certain particular embodiments, the host cell is a hybridoma cell. In certain embodiments, the antigen-binding fragment of any of the aforementioned antibodies is selected from F(ab′)₂, Fab′, Fab, Fd, and Fv. In other particular embodiments, the antigen-binding fragment is of human, mouse, chicken, or rabbit origin. In another embodiment, the antigen-binding fragment is a single chain Fv (scFv). Also provided herein in another embodiment, is a composition comprising any of the aforementioned antibodies, or antigen-binding fragment thereof, and a pharmaceutically suitable carrier. In another embodiment, a method of altering an immune response in a subject is provided wherein the method comprises administering to the subject the composition comprising the antibody, or antigen binding fragment thereof, thereby altering the immune response of the subject. In a particular embodiment, altering the immune response comprises suppressing the immune response. In one embodiment, a method is provided for treating an immunological disease or disorder in a subject comprising administering to the subject the composition comprising the antibody, or antigen binding fragment thereof and a pharmaceutically acceptable carrier. In a particular embodiment, the immunological disease or disorder is an autoimmune disease or an inflammatory disease. In another particular embodiment, the immunological disease or disorder is multiple sclerosis, rheumatoid arthritis, a spondyloarthropathy, systemic lupus erythematosus, graft versus host disease, an antibody-mediated inflammatory or autoimmune disease or disorder, sepsis, diabetes, psoriasis, atherosclerosis, Sjogren's syndrome, progressive systemic sclerosis, scleroderma, acute coronary syndrome, ischemic reperfusion, Crohn's Disease, endometriosis, glomerulonephritis, myasthenia gravis, idiopathic pulmonary fibrosis, asthma, acute respiratory distress syndrome (ARDS), vasculitis, or inflammatory autoimmune myositis. In yet another particular embodiment, the spondyloarthropathy is selected from ankylosing spondylitis, reactive arthritis, enteropathic arthritis associated with inflammatory bowel disease, psoriatic arthritis, isolated acute anterior uveitis, undifferentiated spondyloarthropathy, Behcet's syndrome, and juvenile idiopathic arthritis. Also provided herein in another embodiment, is a method of manufacture for producing the antibody, or antigen-binding fragment thereof, described above and herein.

In another embodiment provided herein is an agent that specifically binds to CD47 and that inhibits binding of a viral CD47-like polypeptide to at least one CD47 ligand. In a particular embodiment, the viral CD47-like polypeptide is a poxvirus CD47-like polypeptide. In yet another specific embodiment, the poxvirus CD47-like polypeptide is a variola CD47-like polypeptide comprising the amino acid sequence set forth in SEQ ID NO:3. In another embodiment, the at least one CD47 ligand is selected from SIRP-α, SIRP-beta 2, thrombospondin-1, αᵥβ₃ integrin, and α₂β₁ integrin. In yet another embodiment, the agent is selected from a small molecule; an aptamer; and a peptide-Fc fusion polypeptide. Also provided is a composition comprising the agent according and a pharmaceutically suitable carrier. In another embodiment, a method of altering an immune response in a subject is provided wherein the method comprises administering to the subject the composition comprising the agent, thereby altering the immune response of the subject. In a particular embodiment, altering the immune response comprises enhancing the immune response.

In another embodiment, a method for identifying an agent that alters immunoresponsiveness of an immune cell is provided wherein the method comprises: (a) contacting (i) a candidate agent; (ii) a viral CD47-like polypeptide; and (iii) a CD47 ligand, under conditions and for a time sufficient to permit interaction between the CD47 ligand and the viral CD47-like polypeptide; (b) determining a level of binding of the viral CD47-like polypeptide to the CD47 ligand in the presence of the candidate agent and comparing a level of binding of the viral CD47-like polypeptide to the CD47 ligand in the absence of the candidate agent, wherein a decrease in the level of binding of the viral CD47-like polypeptide to the CD47 ligand in the presence of the candidate agent indicates that the candidate agent inhibits binding of the viral CD47-like polypeptide to the CD47 ligand; (c) contacting (i) the candidate agent; (ii) a CD47 ligand; and (iii) an immune cell that expresses CD47, under conditions and for a time sufficient to permit interaction between a CD47 ligand and CD47; and (d) determining a level of binding of the CD47 ligand to the immune cell in the presence of the candidate agent and comparing a level of binding of the CD47 ligand to the immune cell in the absence of the candidate agent, wherein a decrease in the level of binding of the CD47 ligand to the immune cell in the presence of the candidate agent indicates that the candidate agent alters immunoresponsiveness of the immune cell. In particular embodiments, the viral CD47-like polypeptide is a poxvirus CD47-like polypeptide. In yet another specific embodiment, the poxvirus CD47-like polypeptide is a variola virus CD47-like polypeptide comprising the sequence set forth in SEQ ID NO:3. In another embodiment of the method, the CD47 ligand is selected from SIRP-α, SIRP-beta 2, thrombospondin-1, αᵥβ₃ integrin, and α₂β₁ integrin.

In another embodiment, a method is provided for treating an immunological disease or disorder in a subject comprising administering to the subject a composition that comprises (a) a pharmaceutically suitable carrier; and either (i) an agent that specifically binds to CD47 and that impairs binding of a viral CD47-like polypeptide to a CD47 ligand or (ii) an agent that specifically binds to a CD47 ligand and specifically impairs binding of a viral CD47-like polypeptide to the CD47 ligand. In a particular embodiment, the viral CD47-like polypeptide is a poxvirus CD47-like polypeptide. In still another particular embodiment, the poxvirus CD47-like polypeptide is a variola virus CD47-like polypeptide comprising the amino acid sequence set forth in SEQ ID NO:3. In one specific embodiment, the agent is selected from an antibody, or antigen-binding fragment thereof; a small molecule; an aptamer; and a peptide-Fc fusion polypeptide. In yet another specific embodiment, the agent that specifically binds to a CD47 ligand is an antibody, or antigen-binding fragment thereof, that specifically binds to the CD47 ligand, wherein the ligand is SIRP-α, SIRP-beta-2, thrombospondin-1, αᵥβ₃ integrin, or α₂β₁ integrin.

Also provided herein in another embodiment, is a method of treating a disease or disorder associated with alteration of at least one of cell migration, cell proliferation, and cell differentiation in a subject, wherein the method comprises administering to the subject a pharmaceutically suitable carrier and either (a) an agent that specifically binds to CD47 and that impairs binding of a viral CD47-like polypeptide to a CD47 ligand or (b) an agent that specifically binds to a CD47 ligand and specifically impairs binding of a viral CD47-like polypeptide to the CD47 ligand. In one certain embodiment, the viral CD47-like polypeptide is a poxvirus CD47-like polypeptide. In another certain embodiment, the poxvirus CD47-like polypeptide is a variola virus CD47-like polypeptide comprising the amino acid sequence set forth in SEQ ID NO:3. In another embodiment, the CD47 ligand is selected from SIRP-α, SIRP-beta 2, thrombospondin-1, αᵥβ₃ integrin, and α₂β₃ integrin. In still another specific embodiment, the disease or disorder is an immunological disease or disorder, a cardiovascular disease or disorder, a metabolic disease or disorder, or a proliferative disease or disorder. In another embodiment, the immunological disease or disorder is an autoimmune disease or an inflammatory disease. In yet certain embodiments, the immunological disease or disorder is multiple sclerosis, rheumatoid arthritis, a spondyloarthropathy, systemic lupus erythematosus, graft versus host disease, an antibody-mediated inflammatory or autoimmune disease or disorder, sepsis, diabetes, psoriasis, atherosclerosis, Sjogren's syndrome, progressive systemic sclerosis, scleroderma, acute coronary syndrome, ischemic reperfusion, Crohn's Disease, endometriosis, glomerulonephritis, myasthenia gravis, idiopathic pulmonary fibrosis, asthma, acute respiratory distress syndrome (ARDS), vasculitis, or inflammatory autoimmune myositis. In a particular embodiment, the spondyloarthropathy is selected from ankylosing spondylitis, reactive arthritis, enteropathic arthritis associated with inflammatory bowel disease, psoriatic arthritis, isolated acute anterior uveitis, undifferentiated spondyloarthropathy, Behcet's syndrome, and juvenile idiopathic arthritis.

In a particular embodiment, the cardiovascular disease or disorder is atherosclerosis, endocarditis, hypertension, or peripheral ischemic disease. In one particular embodiment, the agent is selected from an antibody, or antigen-binding fragment thereof; a small molecule; an aptamer; and a peptide-Fc fusion polypeptide. In another particular embodiment, the agent that specifically binds to a CD47 ligand is an antibody, or antigen-binding fragment thereof, that specifically binds to the CD47 ligand, wherein the ligand is SIRP-α, SIRP-beta-2, thrombospondin-1, αᵥβ₃ integrin, or α₂β₁ integrin. In one specific embodiment, the antibody specifically binds to SIRP-α; in another specific embodiment, an antibody, or antigen-binding fragment thereof, specifically binds SIRP-beta-2; in another specific embodiment, an antibody, or antigen-binding fragment thereof, specifically binds thrombospondin-1; in another specific embodiment, an antibody, or antigen-binding fragment thereof, specifically binds αᵥβ₃ integrin; and in yet another specific embodiment, an antibody, or antigen-binding fragment thereof, specifically binds α₂β₁.

In another embodiment, a method of activating an immune cell is provided wherein the method comprises contacting the immune cell with either (a) an agent that specifically binds to CD47 and that impairs binding of a viral CD47-like polypeptide to a CD47 ligand, or (b) an agent that specifically binds to a CD47 ligand and that impairs binding of a viral CD47-like polypeptide to a CD47 ligand under conditions and a time sufficient to permit the immune cell and the agent to interact, wherein the CD47 ligand is present on the cell surface of the immune cell, thereby activating the immune cell. In one specific embodiment, the CD47 ligand is selected from SIRP-α, SIRP-beta-2, thrombospondin-1, αᵥβ₃ integrin, and α₂β₁ integrin. In a particular specific embodiment, the CD47 ligand is SIRP-α. In another embodiment, activating the immune cell comprises inhibiting Fc-mediated cytokine production. In a certain specific embodiment, inhibiting Fc-mediated chemokine production comprises inhibiting immune complex-induced cytokine production. In another specific embodiment, activating the immune cell comprises inhibiting Fc-mediated chemokine production. In certain embodiments, the immune cell is a dendritic cell, a monocyte, a granulocyte, or a bone marrow stem cell. In other specific embodiments, the agent is selected from an antibody, or antigen-binding fragment thereof; a small molecule; an aptamer; and a peptide-Fc fusion polypeptide. In yet other specific embodiments, the agent that specifically binds to a CD47 ligand is an antibody, or antigen-binding fragment thereof, that is specific for the CD47 ligand, wherein the ligand is SIRP-α, SIRP-beta-2, thrombospondin-1, αᵥβ₃ integrin, or α₂β₁ integrin.

Also provided in another embodiment, is a method of manufacture for producing an agent that suppresses immunoresponsiveness of an immune cell, comprising: (a) identifying an agent that alters immunoresponsiveness of an immune cell, wherein the step of identifying comprises: (I) contacting (i) a candidate agent; (ii) a viral CD47-like polypeptide; and (iii) a CD47 ligand, under conditions and for a time sufficient to permit interaction between the CD47 ligand and the viral CD47-like polypeptide; and (II) determining a level of binding of the viral CD47-like polypeptide to the CD47 ligand in the presence of the candidate agent and comparing a level of binding of the viral CD47-like polypeptide to the CD47 ligand in the absence of the candidate agent, wherein a decrease in the level of binding of the viral CD47-like polypeptide to the CD47 ligand in the presence of the candidate agent indicates that the candidate agent inhibits binding of the viral CD47-like polypeptide to the CD47 ligand; (III) contacting (i) the candidate agent; (ii) a CD47 ligand; and (2) an immune cell that expresses CD47, under conditions and for a time sufficient to permit interaction between the CD47 ligand and CD47; and (IV) determining a level of binding of the CD47 ligand to the immune cell in the presence of the candidate agent and comparing a level of binding of the CD47 ligand to the immune cell in the absence of the candidate agent, wherein a decrease in the level of binding of the CD47 ligand to the immune cell in the presence of the candidate agent indicates that the candidate agent alters immunoresponsiveness of the immune cell; and (b) producing the agent identified in step (a). In one embodiment, the viral CD47-like polypeptide is a poxvirus CD47-like polypeptide. In a specific embodiment, the poxvirus CD47-like polypeptide is a variola virus CD47-like polypeptide comprising the amino acid sequence set forth in SEQ ID NO:3. In another particular embodiment, the CD47 ligand is selected from SIRP-α, SIRP-beta 2, thrombospondin-1, αᵥβ₃ integrin, and α₂β₁ integrin. In one specific embodiment, the agent is selected from an antibody, or antigen-binding fragment thereof; a small molecule; an aptamer; and a peptide-Fc fusion polypeptide. In another specific embodiment, the agent is an antibody, or antigen-binding fragment thereof. In still yet another embodiment, the antibody, or antigen-binding fragment thereof, is selected from an antibody, or antigen-binding fragment thereof, that specifically binds to SIRP-α, SIRP-beta-2, thrombospondin-1, αᵥβ₃ integrin, or α₂β₁ integrin.

In one embodiment, a fusion polypeptide comprising an extracellular domain of a viral CD47 polypeptide fused to an Fc polypeptide is provided. In a particular embodiment, the viral CD47 polypeptide is a poxvirus CD47-like polypeptide. In yet another particular embodiment, the poxvirus CD47-like polypeptide is selected from a myxoma, a orthopoxvirus, a chordopoxvirus, a capripoxvirus, a leporipoxvirus, a suipoxvirus, a yatapoxvirus, and a deerpox virus. In another embodiment, the Fc polypeptide is a human IgG1 Fc polypeptide, or variant thereof.

As used herein, the term “isolated” means that a material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such a nucleic acid could be part of a vector and/or such nucleic acid or polypeptide could be part of a composition, and still be isolated in that the vector or composition is not part of the natural environment for the nucleic acid or polypeptide. The term “gene” means the segment of DNA involved in producing a polypeptide chain; it includes regions preceding and following the coding region “leader and trailer” as well as intervening sequences (introns) between individual coding segments (exons). Amino acids may be referred to herein according to the single letter and three letter codes, which are common textbook knowledge in the art, and therefore with which a person skilled in the art is familiar. The term “fusion polypeptide” used herein may also be used interchangeably with “fusion protein,” and unless specifically indicated otherwise, the two terms are not meant to indicate molecules that have distinguishable properties or characteristics.

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells and equivalents thereof known to those skilled in the art, and so forth. The term “comprising” (and related terms such as “comprise” or “comprises” or “having” or “including”) is not intended to exclude that, for example, any composition of matter, composition, method, or process, or the like, described herein may “consist of” or “consist essentially of” the described features.

All U.S. patents, U.S. patent application publications, U.S. patent applications, foreign patents, foreign patent applications, and non-patent publications referred to in this specification and/or listed in the Application Data Sheet are incorporated herein by reference, in their entirety.

### BRIEF DESCRIPTION OF THE DRAWINGS

FIG. 1 presents a schematic of human CD47 expressed on a cell surface and a viral CD47-like polypeptide expressed on a cell surface.

FIG. 2 presents an alignment of human CD47 (SEQ ID NO:9) (GenBank Accession No. NP_{—}001768.1) with vCD47 (SEQ ID NO:3) from Variola minor virus. The asterisk (*) indicates amino acid identity between the sequences and “:” indicates amino acid similarity. The cysteine residues that form an intramolecular disulfide bond are indicated in boldface type. The key indicates that the amino acids of the signal peptide of each polypeptide are italicized; the Ig domain cysteine loop is indicated by a single underline; the intracellular portions of the transmembrane portion (shaded in gray) and the intracytoplasmic tail are indicated by double underlining; and the extracellular portion of the transmembrane portion of the CD47 and viral CD47-like polypeptides are indicated by bold underlining.

FIGS. 3A and 3B show that a human CD47 extracellular domain-Fc fusion polypeptide (CD47-hFc) inhibits Staphylococcus aureus Cowan strain (SAC)-induced TNF-alpha (TNF-α) (FIG. 3A) and IL-12p70 (FIG. 3B) production in human dendritic cells. Eight day-old human monocyte-derived DC (2×10⁴ cells/96-well) from three donors were treated with the indicated concentrations of hCD47-Fc and human IgG (IgG) in the presence of IFN-γ. Then, the dendritic cells were stimulated with 0.01% SAC. TNF-α and IL12p70 concentrations were determined in supernatants removed from the cells after 18 h.

FIG. 4 illustrates the concentration of TNF-α in supernatants from DCs stimulated with HuS-SAC (human sera-SAC). Eight day-old human monocyte-derived DC (2×10⁴ cells/96-well) were treated with the indicated concentrations of hCD47-Fc and hFc-Stub in the presence of IFN-γ. Then, DC were stimulated with 0.01% PBS-SAC or 0.01% HuS-SAC. TNF-α was determined in supernatants removed from the cultures after 18 h.

FIG. 5 the concentration of IL-23 in supernatants from DCs stimulated with HuS-SAC (human sera-SAC). Eight day-old human monocyte-derived DC (2×10⁴ cells/96-well) were treated with the indicated concentrations of hCD47-Fc and hFc-Stub (lacking the CD47 moiety) in the presence of IFN-γ. Then, DC were stimulated with 0.01% FBS (fetal bovine sera)-SAC or 0.01% HuS-SAC. IL-23 was determined in supernatants removed from the cultures after 18 h.

FIG. 6 illustrates that a human CD47 extracellular domain-Fc polypeptide fusion polypeptide (hCD47-Fc) inhibited IgG-mediated TNF-α production in human dendritic cells. Human dendritic cells were combined with hCD47-Fc or hFc-Stub in 96-well plates that had previously been coated with anti-human Fc donkey IgG (right side: immobilized hCD47-Fc) or human dendritic cells were combined with hCD47-Fc or hFc-Stub in the absence of donkey IgG (left side: soluble hCD47-Fc). The dendritic cells were stimulated with 0.1 ng/ml FSL-1 and the presence of TNF-α in the cell supernatants was determined.

FIG. 7 shows human CD47 extracellular domain-Hac polypeptide (non-immunoglobulin) fusion polypeptide (hCD47-Hac) inhibited IgG-mediated TNF-α production in human dendritic cells. Human dendritic cells were combined with hCD47-Hac or a control construct (gluc-Hac: Gaussia luciferase-Hac) in 96-well plates that had previously been coated with mouse IgG (right side: 50 μg/ml mouse IgG) or human dendritic cells were combined with hCD47-Hac or gluc-Hac in the absence of mouse IgG (left side: no mouse IgG). The dendritic cells were stimulated with 0.1 ng/ml FSL-1 and the presence of TNF-α in the cell supernatants was determined.

FIG. 8 presents the effect of a murine CD47 extracellular domain-murine Fc polypeptide fusion protein on cytokine production of stimulated murine dendritic cells. After activating 9 day-old bone marrow-derived DC (2×10⁴/well) with IFN-γ (1000 U/ml) overnight, cells were treated for 1 h with the indicated concentrations of mCD47-Fc and mIgG2a. Then, DC were stimulated with 0.01% IgG2a-SAC. The level of TNF-α and IL-12 was determined in supernatants removed from the cells after 18 h.

FIG. 9 presents the effect of mCD47-Fc in a murine model of collagen antibody induced arthritis (CAIA). PBS, mIgG (500 μg), or mCD47-mFc (500 μg) was administered intravenously to male DBA/1J mice on days 0, 2, 4, 6, and 8. 4 mg of ArthitoMAB™ antibody cocktail was administered intravenously on Day 0. Mice were boosted intraperitoneally with 50 μg of LPS on days 6 and 13. Total disease scores for individual mice are shown for days 8, 13, and 18.

### DETAILED DESCRIPTION

Described herein are fusion polypeptides that comprise a CD47 extracellular domain polypeptide, or a variant thereof, fused to an immunoglobulin Fc polypeptide, including a human IgG1 Fc polypeptide. The CD47-Fc polypeptide fusion polypeptides described herein are capable of altering the immunoresponsiveness of an immune cell. The CD47-Fc polypeptide fusion proteins described herein, may modulate or alter the immune response of a host, and may particularly inhibit, suppress, or decrease the extent of, an immune response exhibited in an immunological disease or disorder, for example, an inflammatory or autoimmune disease or disorder. In certain embodiments, the CD47-Fc polypeptide fusion proteins inhibit cytokine and/or chemokine production by immune cells, which reduces the inflammatory response of the immune cells.

The CD47-Fc polypeptide fusion proteins described herein may be capable of interacting with a CD47 ligand that is present on the cell surface of an immune cell, and thereby stimulating or inducing the CD47 ligand to deliver a signal to the immune cell, resulting in activation of one or more biological functions of the cell. Such CD47 ligands include but are not limited to SIRP-α, SIRP-beta-2, thrombospondin-1, αᵥβ₃ integrin, and α₂β₁ integrin, which are described in greater detail herein.

In particular embodiments, the fusion polypeptides are capable of inhibiting Fc-mediated activation of an immune cell, and thus may inhibit cytokine and/or chemokine production of immune cells, particularly immune cells that express a CD47 ligand on the cell surface. An Fc-mediated activity includes immune complex-induced immunoresponsiveness of an immune cell. As described herein, the presence of an immune complex (i.e., an antigen-antibody complex) interacting with the immune cell activates the immune cell and induces cytokine production by the immune cell, which can be inhibited by the CD47-Fc fusion polypeptides described herein. Immune complexes can damage tissue by triggering Fc-receptor mediated inflammation, a process implicated in several human immunological diseases, for example, systemic lupus erythematosus, rheumatoid arthritis, and Sjoergen's syndrome. Thus, the fusion polypeptides described herein may be useful for altering immunoresponsiveness of an immune cell and thereby may be useful for treating or preventing an immunological disease or disorder, cardiovascular disease or disorder, metabolic disease or disorder, or a proliferative disease or disorder.

The extracellular CD47 domain moiety of the fusion polypeptide includes a CD47 extracellular domain variant. An exemplary variant comprises the extracellular domain of human CD47 that is truncated or that comprises at least one substitution, insertion, or deletion of an amino acid in the extracellular domain. In other certain embodiments, two fusion polypeptides form a dimer. The two fusion polypeptides dimerize, at least in part, via one or more interchain disulfide bonds between the Fc polypeptide moieties of each of two fusion polypeptides. As described in greater detail herein a fusion polypeptide may also dimerize via the CD47 moieties of each of the two fusion polypeptides, via the formation of an interchain disulfide bond between cysteine residues of the CD47 moieties.

As described herein, a CD47-Fc polypeptide fusion polypeptide may be useful for treating or preventing, inhibiting, slowing the progression of, or reducing the symptoms associated with, an immunological disease or disorder, a cardiovascular disease or disorder, a metabolic disease or disorder, or a proliferative disease or disorder. An immunological disorder includes an inflammatory disease or disorder and an autoimmune disease or disorder. While inflammation or an inflammatory response is a host's normal and protective response to an injury, inflammation can cause undesired damage. For example, atherosclerosis is, at least in part, a pathological response to arterial injury and the consequent inflammatory cascade. A cardiovascular disease or disorder that may be treated, which may include a disease or disorder that is also considered an immunological disease/disorder, includes for example, atherosclerosis, endocarditis, hypertension, or peripheral ischemic disease. A metabolic disease or disorder includes diabetes, obesity, and diseases and disorders associated with abnormal or altered mitochondrial function.

An immunological disease or disorder may be an autoimmune disease or an inflammatory disease. In certain embodiments, the immunological disease or disorder is multiple sclerosis, rheumatoid arthritis, a spondyloarthropathy, systemic lupus erythematosus, graft versus host disease, an antibody-mediated inflammatory or autoimmune disease or disorder, sepsis, diabetes, psoriasis, atherosclerosis, Sjogren's syndrome, progressive systemic sclerosis, scleroderma, acute coronary syndrome, ischemic reperfusion, Crohn's Disease, endometriosis, glomerulonephritis, myasthenia gravis, idiopathic pulmonary fibrosis, asthma, acute respiratory distress syndrome (ARDS), vasculitis, or inflammatory autoimmune myositis. A spondyloarthropathy includes, for example, ankylosing spondylitis, reactive arthritis, enteropathic arthritis associated with inflammatory bowel disease, psoriatic arthritis, isolated acute anterior uveitis, undifferentiated spondyloarthropathy, Behcet's syndrome, and juvenile idiopathic arthritis. The fusion polypeptides described herein may also be useful for treating a cardiovascular disease or disorder, such as atherosclerosis, endocarditis, hypertension, or peripheral ischemic disease. In other certain embodiments, the fusion polypeptides described herein may be used for treating a proliferative disease, such as cancer. A cancer or malignancy includes, but is not limited to, a leukemia (e.g., B-cell chronic lymphocytic leukemia), lymphoma, breast cancer, renal cancer, and ovarian cancer.

Also described herein are methods for identifying and making CD47 extracellular domain variants that are capable of altering an immune response and the immunoresponsiveness of an immune cell. Such methods include determining interactions among CD47 ligands and a viral virulence factor that is a CD47 ortholog. Viruses have evolved numerous mechanisms to evade detection and elimination by the immune system of an infected host by encoding proteins that are viral homologues of cell cytokines and chemokines and their receptors. For example, the genomes of poxviruses encode a soluble viral tumor necrosis factor (TNF) receptor, which binds to and inhibits the inflammation-inducing cytokine, TNF. Other cellular polypeptides targeted by viral polypeptides or that are homologues of viral polypeptides include interleukin 1, various chemokines, CD30, and CD47. Genes that encode a CD47-like polypeptide have been identified in certain poxvirus family members including orthopoxvirus, capripoxvirus, leporipoxvirus, suipoxvirus, and yatapoxvirus (see, e.g., Cameron et al., Virology 264:298-318 (1999); Cameron et al., Virology 337:55-67 (2005); Alfonso et al., J. Virol. 79:966-977 (2005); Seet et al., Annu. Rev. Immunol. 21:377-423 (2003)). This CD47-like polypeptide virulence factor is referred to herein as viral CD47 (vCD47). In certain embodiments, as described in greater detail herein, the fusion polypeptides comprising CD47 extracellular domain, or a variant thereof, alter an immune response by interacting with at least one CD47 ligand in the same manner as viral CD47 interacts with the CD47 ligand.

### CD47

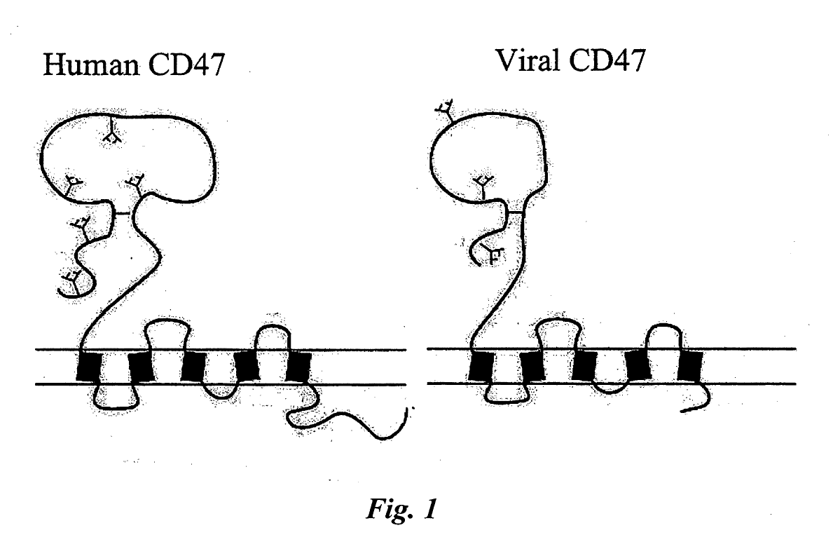

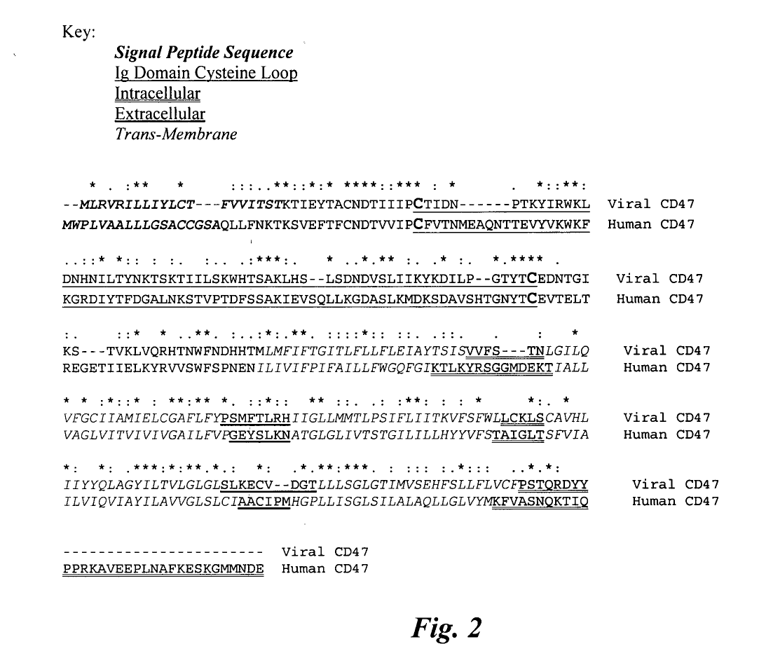

CD47 (also known in the art as integrin-associated protein (IAP)) is a mammalian cell surface receptor glycoprotein of approximately 50 kiloDaltons (kD). Structurally, CD47 has an extracellular Ig-like domain, five transmembrane portions, and a short cytoplasmic domain (see FIG. 1 and FIG. 2). CD47 is associated with β₃ and β₁ integrins, primarily αᵥβ₃ integrin, which is the vitronectin receptor, and with α₂β₁, which is a collagen and laminin receptor. CD47 is involved with several cellular processes including, for example, neutrophil phagocytosis, T cell activation, T and B cell apoptosis, platelet activation, stroma-supported erythropoiesis, immune cell (e.g., neutrophils) transmigration, and cell adhesion (see, e.g., Latour et al., J. Immunol. 167:2547-54 (2001); Fukunaga et al., J. Immunol. 172:4091-99 (2004); Parkos et al., J. Cell Biol. 132:437-50 (1996); Liu et al., J. Biol. Chem. 277:10028-36 (2002); Lamy et al., J. Biol. Chem. 278:23915-21 (2003); Cooper et al., Proc. Natl. Acad. Sci. USA 92:3978-82 (1995); Motegi et al., EMBO J. 22:2634-44 (2003); Manna et al., Cancer Res. 64:1026-36 (2004); PCT International Publication No. WO 99/40940; PCT International Publication No. WO 97/27873).

Human CD47 may be present on the surface of a cell as one of four major isoforms (Reinhold et al., J. Cell Sci. 108:3419-25 (1995)). Exemplary CD47 nucleotide sequences and the amino acid sequences of the encoded CD47 polypeptides are provided in publicly available databases (see, e.g., GenBank Accession No. NP_{—}001768.1 (SEQ ID NO:9), isoform 1 (see also GenBank Accession No. NM_{—}001777.3 (SEQ ID NO:14)); GenBank Accession No. NP_{—}942088.1 (SEQ ID NO:15), isoform 2 (see also GenBank Accession No. NM_{—}198793.2 (SEQ ID NO:16)); GenBank Accession No. NP_{—}001020250.1 (SEQ ID NO:17), isoform 3 (see also GenBank Accession No. NM_{—}001025079.1 (SEQ ID NO:13)); see also, e.g., GenBank Accession Nos. BT006907.1; BC012884.1; BC010016.2; BC037306.1; BC045593.1; BC053959.1; and BC042889.1). The isoforms are splice variants mapping in the intracytoplasmic domain (see, e.g., Lindberg et al., J. Cell Biol. 123:485-96 (1993). Nucleotide sequences that encode CD47 of other mammals, including murine and rat CD47, and CD47 polypeptide sequences of other mammals are also known in the art and readily available to persons skilled in the art in sequence databases (e.g., GenBank, Swissprot, and the like).

Each of the exemplary CD47 sequences provided herein may comprise a nucleotide sequence that encodes a signal peptide. By way of example, the signal peptide of certain human CD47 isoforms is reported to be eighteen amino acids (see, e.g., SEQ ID NO:11). Signal peptides are not exposed on the cell surface of a secreted or transmembrane protein because either the signal peptide is cleaved during translocation of the protein or the signal peptide remains anchored in the outer cell membrane (such a peptide is also called a signal anchor) (see, e.g., Nielsen et al., Protein Engineering 10:1-6 (1997); Nielsen et al., in J. Glasgow et al., eds., Proc. Sixth Int. Conf. on Intelligent Systems for Molecular Biology, 122-30 (AAAI Press 1998)). The signal peptide sequence of CD47 is believed to be cleaved from the precursor CD47 polypeptide in vivo. Cleavage of a signal peptide, whether the signal peptide is a specific CD47 signal peptide or a heterologous signal peptide, may be imprecise such that the N-terminal amino acid residue may vary.

CD47 is expressed by cells in many different tissues. CD47 is expressed on most, if not all, hematopoietic cells, including thymocytes, T and B cells, monocytes, platelets, and erythrocytes. CD47 is also expressed on epithelia cells, endothelial cells, fibroblasts, sperm, certain tumor cell lines, mesenchymal cells, and certain neuronal cells.

### CD47 Fusion Polypeptides

CD47 Extracellular Domain and Variants Thereof

In one embodiment, a CD47 fusion polypeptide comprises a CD47 extracellular domain variant fused to a moiety capable of multimer formation (e.g., dimer formation), including for example, an Fc polypeptide. Described herein are CD47 fusion polypeptides that comprise the extracellular domain of CD47, or a variant thereof, fused to an Fc polypeptide (and variants thereof), as described herein, and that may be used for altering the immunoresponsiveness of an immune cell and for treating immunological diseases and disorders. Alternatively, a fusion polypeptide comprising the extracellular domain of CD47 may be fused to another moiety that is capable of multimer formation. In other particular embodiments, the extracellular domain of CD47 may be fused to a heterologous moiety, wherein that moiety is incapable of multimer formation with another CD47 fusion polypeptide comprising the same moiety. In yet another embodiment, a CD47 fusion polypeptide comprising the extracellular domain of CD47 fused to a first heterologous polypeptide such as a first Fc polypeptide, may be combined with a second CD47 fusion polypeptide comprising the extracellular domain of CD47 fused to a second heterologous polypeptide, such as a second Fc polypeptide, such that the two different fusion polypeptides form heterodimers or heteromultimers.

An Fc polypeptide, or portion thereof (such as at least one immunogloblulin constant region domain, for example, the CH2 domain or CH3 domain) when fused to a peptide or polypeptide of interest acts, at least in part, as a vehicle or carrier moiety that prevents degradation and/or increases half-life, reduces toxicity, reduces immunogenicity, and/or increases biological activity of the peptide such as by forming dimers or other multimers (see, e.g., U.S. Pat. Nos. 6,018,026; 6,291,646; 6,323,323; 6,300,099; 5,843,725). (See also, e.g., U.S. Pat. No. 5,428,130; U.S. Pat. No. 6,660,843; U.S. Patent Application Publication Nos. 2003/064480; 2001/053539; 2004/087778; 2004/077022; 2004/071712; 2004/057953/ 2004/053845/ 2004/044188; 2004/001853; 2004/082039). Alternative moieties to an immunoglobulin constant region, such as an Fc polypeptide, that may be linked or fused to a CD47 extracellular domain and that contribute to retention of the capability of the CD47 moiety to alter the immunoresponsiveness of an immune cell include, for example, a linear polymer (e.g., polyethylene glycol, polylysine, dextran, etc.; see, for example, U.S. Pat. No. 4,289,872; International Patent Application Publication No. WO 93/21259); a lipid; a cholesterol group (such as a steroid); a carbohydrate or oligosaccharide. In addition, also contemplated and described herein, a CD47 moiety from one species may be fused to an Fc polypeptide (or other heterologous polypeptide moiety) derived from a different species.

In other certain particular embodiments, for example, when more rapid clearance or increased half life of a CD47 extracellular domain (or dimer or other multimer thereof) may be desirable, the CD47 extracellular domain as a monomer or as a dimer or other multimeric form may be used. Thus in these embodiments, the CD47 extracellular domain is not fused to a heterologous moiety.

Isoforms of CD47 have been identified, and the amino acid sequences of several different isoforms have been deduced. The isoforms of CD47 differ from each other primarily in the amino acid sequence of the intracellular portion of CD47. The amino acid sequences of the extracellular portion (also herein called the extracellular domain) of different CD47 isoforms, such as the exemplary isoforms described herein, are highly similar, and in certain exemplary CD47 polypeptides, the amino acid sequences of the extracellular domains of different CD47 isoforms are identical.

In one embodiment, an exemplary fusion polypeptide comprises the extracellular domain of human CD47 fused to a human IgG1 Fc polypeptide. Typically, in the immunoglobulin art, an Fc polypeptide comprises the hinge region that is between the CH1 and CH2 domains. The hinge region comprises cysteine residues that form interchain disulfide bonds between two immunoglobulin chains (one cysteine residue of a heavy chain forms a disulfide bond with a cysteine residue in the light chain and the remaining cysteine residues in the hinge contribute to disulfide bond formation between two heavy chains). In human IgG1 immunoglobulins, the hinge portion of the constant region has three cysteine residues. In certain embodiments, the Fc polypeptides described herein comprise a substitution or a deletion of a cysteine residue in the hinge region. In a particular embodiment, the substituted or deleted cysteine residue is the cysteine residue most proximal to the amino terminus of the hinge region of the Fc portion of a wildtype human IgG1 immunoglobulin. The Fc polypeptide may further comprise a substitution or deletion of an aspartate residue immediately adjacent to the cysteine residue most proximal to the amino terminus of the hinge region of the Fc portion of a wildtype human IgG1 immunoglobulin. The amino acid sequences of the hinge region from IgG1 immunoglobulins, and other human immunoglobulins as well as from other species, are readily available in public databases and also are described in Kabat et al. (in Sequences of Proteins of Immunological Interest, 4th ed (U.S. Dept. of Health and Human Services, U.S. Government Printing Office, 1991); also available by license via the Internet).

An exemplary amino acid sequence of such a fusion polypeptide is provided in SEQ ID NO:2. In certain embodiments, the fusion polypeptide is a variant of the amino sequence set forth in SEQ ID NO:2 and comprises an amino acid sequence at least 65%-75%, 75%-80%, 80-85%, 85%-90%, or 95%-99% identical (or to any percent identity not specifically enumerated between 70% to 100%) to the amino acid sequence set forth in SEQ ID NO:2. A CD47-Fc polypeptide fusion protein variant may comprise one or more substitutions, deletions, or insertions of an amino acid in the CD47 moiety of the protein and/or one or more substitutions, deletions, or insertions of an amino acid in the Fc polypeptide moiety. Exemplary variants are described in further detail herein. The CD47-Fc polypeptide and variants thereof retain the capability to alter (i.e., increase or decrease in a statistically significant or biologically significant manner) the immunoresponsiveness of an immune cell.

In other embodiments, the CD47 fusion proteins described herein include fusion proteins that have been modified. By way of example, one or more amino acids of either the CD47 moiety or the heterologous moiety, such as the Fc polypeptide in a CD47-Fc fusion polypeptide, may be pegylated or may be glycosylated. Pegylation is the process by which polyethylene glycol (PEG) chains are attached to a polypeptide. In certain instances, pegylation increases the circulating half-life and reducing clearance of a polypeptide. Methods for pegylating proteins and peptides are understood in the art (see, e.g., Harris et al., Nature Reviews (Drug Discovery) 2:214-21 (2003); U.S. Pat. No. 5,770,577; International Patent Application Publication WO 92/16221).

Altering immunoresponsiveness of the immune cell includes any one or more of altering immune cell migration; inhibiting production of at least one cytokine, including but not limited to, TNF-α, IL-12, IL-23, IFN-γ, GM-CSF, and IL-6; inhibiting production of a chemokine, including but not limited to MIP-1α; inhibiting maturation of an immune cell such as a dendritic cell; impairing development of a naïve T cell into a Th1 effector cell; and suppressing a proinflammatory response by the immune cell. In another embodiment, the fusion polypeptide alters immunoresponsiveness of an immune cell by interacting with (i.e., binding to) a cell surface receptor that is a CD47 ligand. Altering immunoresponsiveness of a cell by the CD47 fusion polypeptides described herein may also include inhibiting (likely competitively inhibiting) binding of at least one CD47 ligand to a cellular CD47 polypeptide (that is, a CD47 full-length polypeptide that is expressed by a cell and that is present on the cell surface of the cell) and thus inhibiting at least one biological function attributable to cellular CD47 activation or stimulation, and/or inhibiting at least one biological function attributable to the CD47 ligand and thus inhibiting at least one biological function attributable to the CD47 ligand activation or stimulation; For example, the CD47-Fc fusion polypeptide may interact with SIRP-α that is present on an immune cell such as a dendritic cell, a monocyte, a macrophage, a granulocyte, or a bone marrow derived stem cell, thereby stimulating or inducing SIRP-α to signal the immune cell. The fusion polypeptides described herein may interfere with or inhibit an Fc-mediated or immune complex induced activity, such as cytokine or chemokine production, by an immune cell, including an immune cell that expresses SIRP-α.

The fusion polypeptides described herein may alter immunoresponsiveness of an immune cell, such as an immune cell that expresses a CD47 ligand. In a certain specific embodiment, the CD47-Fc fusion polypeptides described herein interact with an immune cell, for example, a dendritic cell, that expresses a CD47 ligand, such as SIRPα, which interaction may result in inhibiting production of at least one cytokine such as TNF-α, IL-12, IL-6, and IL-23. The CD47-Fc fusion polypeptides may activate an immune cell, such as an immune cell that has SIRPα present on the cell surface, by interacting with the immune cell and with SIRPα. Such activation or stimulation of the immune cell may comprise inhibiting an Fc-mediated biological effect (or activity), including cytokine and/or chemokine production by the immune cell. In other embodiments, activation or stimulation of the immune cell may comprise inhibiting an immune-complex induced effect, such as inhibiting immune complex-induced cytokine and/or chemokine production. Thus, cytokine production may be inhibited by interfering with or inhibiting the interaction between an immune complex and the immune cell, which can reduce or inhibit production of one or more cytokines by the immune cell. The CD47-Fc polypeptide, and variants thereof, also may retain the capability to bind at least one CD47 ligand, for example, SIRP-α, SIRP-beta-2, thrombospondin-1, αᵥβ₃ integrin, and α₂β₁ integrin.

A person skilled in the art will readily appreciate that CD47-Fc polypeptide fusion proteins can be made using the extracellular domain of the CD47 polypeptide from a species and fused to an Fc polypeptide from an immunoglobulin from the same or different species. By way of example, the extracellular domain of a murine CD47 is fused to a murine IgG Fc polypeptide, such as an Fc polypeptide derived from an IgG2a or IgG2b immunoglobulin. As described in further detail herein, a CD47 extracellular domain may be fused to other moieties, including other polypeptides.

In certain embodiments, a CD47 fusion polypeptide comprises the extracellular domain of CD47, including the signal peptide (SEQ ID NO:18), such that the extracellular portion of CD47 is typically 142 amino acids in length, and has the amino acid sequence set forth in SEQ ID NO:11. The fusion polypeptides described herein also include CD47 extracellular domain variants that comprise an amino acid sequence at least 65%-75%, 75%-80%, 80-85%, 85%-90%, or 95%-99% (or any percent identity not specifically enumerated between 65% to 100%), which variants retain the capability to alter the immunoresponsiveness of an immune cell and/or to bind at least one CD47 ligand.

In certain embodiments, the signal peptide amino acid sequence may be substituted with a signal peptide amino acid sequence that is derived from another polypeptide (e.g., for example, an immunoglobulin or CTLA4). For example, unlike full-length CD47, which is a cell surface polypeptide that traverses the outer cell membrane, the CD47 fusion polypeptides are secreted; accordingly, a polynucleotide encoding a CD47 fusion polypeptide may include a nucleotide sequence encoding a signal peptide that is associated with a polypeptide that is normally secreted from a cell.

In other embodiments, the CD47 fusion polypeptide comprises an extracellular domain of CD47 that lacks the signal peptide. In an exemplary embodiment, the CD47 extracellular domain lacking the signal peptide has the amino acid sequence set forth in SEQ ID NO:1 (124 amino acids). As described herein, signal peptides are not exposed on the cell surface of a secreted or transmembrane protein because either the signal peptide is cleaved during translocation of the protein or the signal peptide remains anchored in the outer cell membrane (such a peptide is also called a signal anchor). The signal peptide sequence of CD47 is believed to be cleaved from the precursor CD47 polypeptide in vivo.

In other embodiments, a CD47 fusion polypeptide comprises a CD47 extracellular domain variant. Such a CD47 fusion polypeptide retains the capability to bind specifically to at least one CD47 ligand, which includes SIRP-α, SIRP-beta 2, thrombospondin-1, αᵥβ₃ integrin, and α₂β₁ integrin, for example. The CD47 extracellular domain variant may have an amino acid sequence that is at least 65%-75%, 75%-80%, 80-85%, 85%-90%, or 95%-99% identical (which includes any percent identity between any one of the described ranges) to SEQ ID NO:1.

In certain particular embodiments, the CD47 extracellular domain variants described herein retain all cysteine residues. For example, cysteine residues that correspond to the cysteine residues at position 15, at position 23, and position 96 of SEQ ID NO:1 are retained. The cysteine residues at position 23 and position 96 correspond to the cysteine residues in the full-length CD47 molecule that typically form an intramolecular (or intrachain) disulfide bond. When the extracellular domain of CD47 comprises the signal peptide, the cysteine residues retained are located at positions 33, 41, and 114 of SEQ ID NO: 11, and the cysteine residues at positions 41 and 114 typically form an intramolecular disulfide bond. Without wishing to be bound by any particular theory, the cysteine residue nearest the amino terminal end of the extracellular CD47 domain, that is, the cysteine residue at position 15 of SEQ ID NO:1 and at position 33 of SEQ ID NO:11 may form an interchain disulfide bond such that the CD47 moieties of two CD47-Fc polypeptide fusion proteins form a CD47 dimer, thus forming a fusion polypeptide dimer.

Without wishing to be bound by theory, the intramolecular disulfide bond appears to contribute to the conformation of the immunoglobulin-like domain structure of CD47 expressed on a cell surface and to the retention of the immunoglobulin-like structure of the CD47 fusion polypeptides described herein. Persons skilled in the art will appreciate that the cysteine residues that typically form an intramolecular disulfide bond in CD47 may be located at positions that differ from the numbered positions of the cysteine residues in SEQ ID NO:1 or SEQ ID NO:11 and that determining the location of corresponding cysteine residues in another amino acid sequence that form an intramolecular disulfide bond in a CD47 polypeptide is well within the routine practice of the skilled artisan using alignment programs and molecular modeling programs described herein and used in the art. Similarly, determining the corresponding amino acids of CD47 molecules compared to particular amino acids provided in the exemplary amino acid sequences described herein can be readily determined using alignment tools described herein and routinely used by persons skilled in the art.

In another embodiment, the CD47 extracellular domain variant comprises a substitution or a deletion of the cysteine residue that is most proximal to the amino terminal end of the CD47 extracellular domain. This cysteine residue is located at a position corresponding to the cysteine residue at position 15 of SEQ ID NO:1 and is located at position 33 of SEQ ID NO:11. In a particular embodiment, the cysteine residue may be substituted with any amino acid, for example the cysteine residue may be substituted with a serine residue. In another particular embodiment, the cysteine residue may be substituted with one, two, or three, or four amino acids. For example, the cysteine residue most proximal to the amino terminal end of CD47 (i.e., the cysteine at position 15 of SEQ ID NO:1 or position 33 of SEQ ID NO:11) may be substituted with a tripeptide that is a potential glycosylation site, for example, a tripeptide that has the sequence Asn-X-Ser wherein X may be any amino acid except cysteine. Substitution or deletion of the cysteine residues most proximal to the amino terminus of the CD47 extracellular domain moiety eliminates the ability of a CD47 extracellular domain moiety of one fusion polypeptide to form a dimer with another CD47 extracellular domain moiety via formation of a disulfide bond between the cysteine residues of the CD47 extracellular domain moieties of two CD47 fusion polypeptides.

The percent identity of the amino acid sequence of a CD47 extracellular domain variant to the amino acid sequence set forth in either SEQ ID NO:1 or SEQ ID NO:11, or the percent identify between a fusion polypeptide comprising the amino acid sequence set forth in SEQ ID NO:2 and another fusion polypeptide, can be readily determined by persons skilled in the art by sequence comparison. As used herein, two amino acid sequences have 100% amino acid sequence identity if the amino acid residues of the two amino acid sequences are the same when aligned for maximal correspondence. Sequence comparisons of polypeptides and polynucleotides (for example, the polynucleotides that encode the polypeptides described herein) can be performed using any method such as those that use computer algorithms well known to persons having ordinary skill in the art. Such algorithms include Align or the BLAST algorithm (see, e.g., Altschul, J. Mol. Biol. 219:555-565, 1991; Henikoff and Henikoff, Proc. Natl. Acad. Sci. USA 89:10915-10919, 1992), which are available at the NCBI website (see [online] Internet at ncbi.nlm.nih.gov/cgi-bin/BLAST). Default parameters may be used. In addition, standard software programs are available, such as those included in the LASERGENE bioinformatics computing suite (DNASTAR, Inc., Madison, Wis.); CLUSTALW program (Thompson et al., Nucleic Acids Res. 22:4673-80 (1991)); and “GeneDoc” (Nicholas et al., EMBNEW News 4:14 (1991)). Other methods for comparing two amino acid sequences by determining optimal alignment are practiced by persons having skill in the art (see, for example, Peruski and Peruski, The Internet and the New Biology: Tools for Genomic and Molecular Research (ASM Press, Inc. 1997); Wu et al. (eds.), “Information Superhighway and Computer Databases of Nucleic Acids and Proteins,” in Methods in Gene Biotechnology, pages 123-151 (CRC Press, Inc. 1997); and Bishop (ed.), Guide to Human Genome Computing, 2nd Ed. (Academic Press, Inc. 1998)).

A CD47 extracellular domain variant may differ from a wildtype CD47 amino acid sequence (such as the amino acid sequence set forth in SEQ ID NO:1 or SEQ ID NO:11) due to an insertion, deletion, addition, and/or substitution of at least one amino acid and may differ due to the insertion, deletion, addition, and/or substitution of at least two, three, four, five, six, seven, eight, nine, or ten amino acids or may differ by any number of amino acids between 10 and 45 amino acids. A CD47 extracellular domain variant includes, for example, a naturally occurring polymorphism (i.e., allelic variant) or a recombinantly manipulated or engineered CD47 extracellular domain variant.

A CD47 extracellular domain variant that differs from the amino acid sequence set forth in SEQ ID NO:1 includes a variant with at least one deletion from either the amino terminal end or carboxy terminal end or from both the amino terminal end and the carboxy terminal end of the CD47 extracellular domain. Such a CD47 extracellular domain variant may also be referred to herein as a truncated CD47 extracellular domain. The truncation may include a deletion of at least 1, 2, 3, 4, 5, 6, 7, 8, 9, 10, 12, or 15-20 amino acids from the amino terminal end or carboxy terminal end of the CD47 extracellular domain or may include a deletion of between 1-20 amino acids from each terminal end. A truncated CD47 extracellular domain may retain the cysteine residues that correspond to residues at positions 23 and 96 of SEQ ID NO:1 (or at positions 41 and 114 of SEQ ID NO:11) so that the intramolecular disulfide bond is formed in the truncated CD47 extracellular domain variant.

A CD47 fusion polypeptide comprising a CD47 extracellular domain variant that retains the capability to bind specifically to at least one CD47 ligand means that the capability of the variant to bind the CD47 ligand is statistically and/or biologically similar to the capability of the wild type CD47 extracellular domain to bind the CD47 ligand. For example, when the binding affinity and/or other kinetic parameters (e.g., Vmax, kₒₙ, k_{off}) of the variant polypeptide and the wildtype (or non-variant) polypeptide are compared, the binding affinity and/or other kinetic parameters are substantially similar (i.e., within experimental error and variation) between the variant and the wild type polypeptides. Alternatively, or in addition to, a CD47 fusion polypeptide comprising a CD47 extracellular domain variant that retains the capability to bind specifically to at least one CD47 ligand has the capability to effect a biological activity or function that occurs when wild-type CD47 extracellular domain binds the CD47 ligand. Exemplary biological activities are described in detail herein.

Assays for assessing whether a CD47 extracellular domain variant folds into a conformation comparable to the non-variant polypeptide or fragment include, for example, the ability of the protein to react with mono- or polyclonal antibodies that are specific for native or unfolded epitopes, the retention of ligand-binding functions, and the sensitivity or resistance of the mutant protein to digestion with proteases (see Sambrook et al., supra). CD47 extracellular domain variants as described herein can be identified, characterized, and/or made according to these methods described herein or other methods known in the art, which are routinely practiced by persons skilled in the art.

A CD47 fusion polypeptide that comprises a CD47 extracellular domain variant that retains the capability to bind to at least one CD47 ligand and/or the capability to alter (i.e., increase or decrease in a statistically significant or biologically significant manner) immunoresponsiveness of an immune cell include variants that contain conservative amino acid substitutions. A variety of criteria known to persons skilled in the art indicate whether an amino acid that is substituted at a particular position in a peptide or polypeptide is conservative (or similar). For example, a similar amino acid or a conservative amino acid substitution is one in which an amino acid residue is replaced with an amino acid residue having a similar side chain. Similar amino acids may be included in the following categories: amino acids with basic side chains (e.g., lysine, arginine, histidine); amino acids with acidic side chains (e.g., aspartic acid, glutamic acid); amino acids with uncharged polar side chains (e.g., glycine, asparagine, glutamine, serine, threonine, tyrosine, cysteine, histidine); amino acids with nonpolar side chains (e.g., alanine, valine, leucine, isoleucine, proline, phenylalanine, methionine, tryptophan); amino acids with beta-branched side chains (e.g., threonine, valine, isoleucine), and amino acids with aromatic side chains (e.g., tyrosine, phenylalanine, tryptophan). Proline, which is considered more difficult to classify, shares properties with amino acids that have aliphatic side chains (e.g., leucine, valine, isoleucine, and alanine). In certain circumstances, substitution of glutamine for glutamic acid or asparagine for aspartic acid may be considered a similar substitution in that glutamine and asparagine are amide derivatives of glutamic acid and aspartic acid, respectively. As understood in the art “similarity” between two polypeptides is determined by comparing the amino acid sequence and conserved amino acid substitutes thereto of the polypeptide to the sequence of a second polypeptide (e.g., using GENEWORKS, Align, the BLAST algorithm, or other algorithms described herein and practiced in the art).

In certain embodiments, a fusion polypeptide comprising a CD47 extracellular domain, or a variant thereof, is recombinantly expressed. For instance, a CD47 extracellular domain, or variant thereof, fused in frame with an Fc polypeptide, as described in detail herein, may be recombinantly expressed. A recombinant expression construct may be prepared for the expression of a fusion polypeptide according to standard techniques and methods practiced by a skilled person in the molecular biology art. In order to obtain efficient transcription and translation, the polynucleotide sequence in each construct should include at least one appropriate expression control sequence (also called a regulatory sequence), such as and leader sequence and particularly a promoter operatively linked to the nucleotide sequence encoding the CD47 extracellular domain, or variant thereof. Alternatively, the at least one expression control sequence, such as a promoter, may be operatively linked to a nucleotide sequence encoding the signal peptide sequence located at the amino terminal end of the CD47 extracellular domain.

Particular methods for producing polypeptides recombinantly are generally well known and routinely used. For example, molecular biology procedures are described by Sambrook et al. (Molecular Cloning, A Laboratory Manual, 2nd ed., Cold Spring Harbor Laboratory, New York, 1989; see also Sambrook et al., 3rd ed., Cold Spring Harbor Laboratory, New York, (2001)). DNA sequencing can be performed as described in Sanger et al. (Proc. Natl. Acad. Sci. USA 74:5463 (1977)) and the Amersham International plc sequencing handbook and including improvements thereto. Recombinant expression of the fusion polypeptides is described in greater detail herein.

Exemplary nucleotide sequences that encode CD47 from which the nucleotide sequence encoding the CD47 extracellular domain portion of a CD47 fusion polypeptide can be readily obtained are provided herein and are readily available from public databases (see, e.g., GenBank Accession Nos. NM_{—}001777.3 (SEQ ID NO:19); NM_{—}198793.2 (SEQ ID NO:16); NM_{—}001025079.1; BT006907.1; BC012884.1; BC010016.2; BC037306.1; BC045593.1; BC053959.1; and BC042889.1). The nucleotide sequence of a CD47 extracellular domain variant can be determined and/or identified by comparing the nucleotide sequence of a polynucleotide encoding the variant with a polynucleotide described herein or known in the art that encodes a CD47 polypeptide using any one of the alignment algorithms described herein and used in the art. The percent identity between two polynucleotides may thus be readily determined. Polynucleotides have 100% nucleotide sequence identity if the nucleotide residues of the two sequences are the same when aligned for maximal correspondence. In particular embodiments, the nucleotide sequence of a CD47 extracellular domain variant-encoding polynucleotide is at least 70%, 75%, 80%, 85%, 90%, 95%, or 98% identical to one or more of the polynucleotide sequences that encode a CD47 extracellular domain, which are described herein. Polynucleotide variants also include polynucleotides that differ in nucleotide sequence identity due to the degeneracy of the genetic code and encode a CD47 extracellular domain having an amino acid sequence disclosed herein or known in the art. A polynucleotide that encodes a CD47 fusion polypeptide as described herein also includes a polynucleotide that is complementary to such a polynucleotide. Certain polynucleotides that encode a CD47 extracellular domain, variant, or fragment thereof may also be used as probes, primers, short interfering RNA (siRNA), or antisense oligonucleotides. Polynucleotides may be single-stranded DNA or RNA (coding or antisense) or double-stranded RNA (e.g., genomic or synthetic) or DNA (e.g., cDNA or synthetic).

Polynucleotide variants may be identified by alignment procedures described herein and also may be identified by hybridization methods. Hybridization of two polynucleotides may be performed using methods that incorporate the use of suitable moderately stringent conditions, for example, pre-washing in a solution of 5×SSC, 0.5% SDS, 1.0 mM EDTA (pH 8.0); hybridizing at 50° C.-70° C., 5×SSC for 1-16 hours; followed by washing once or twice at 22-65° C. for 20-40 minutes with one or more each of 2×, 0.5×, and 0.2×SSC containing 0.05-0.1% SDS. For additional stringency, conditions may include a wash in 0.1×SSC and 0.1% SDS at 50-60° C. for 15 minutes. As understood by persons having ordinary skill in the art, variations in stringency of hybridization conditions may be achieved by altering the time, temperature, and/or concentration of the solutions used for pre-hybridization, hybridization, and wash steps. Suitable conditions may also depend in part on the particular nucleotide sequences of the probe used (i.e., for example, the guanine plus cytosine (G/C) versus adenine plus thymidine (A/T) content). Accordingly, a person skilled in the art will appreciate that suitably stringent conditions can be readily selected without undue experimentation when a desired selectivity of the probe is identified.

CD47 extracellular domain variants may be readily prepared by genetic engineering and recombinant molecular biology methods and techniques. Analysis of the primary and secondary amino acid sequence of a CD47 polypeptide and of the CD47 extracellular domain and computer modeling of same to analyze the tertiary structure of the polypeptide may aid in identifying specific amino acid residues that can be substituted, added, or deleted without altering the structure and as a consequence, potentially the function, of the CD47 polypeptide. Modification of a polynucleotide, such as DNA, encoding a CD47 extracellular domain variant may be performed by a variety of methods, including site-specific or site-directed mutagenesis of the DNA, which methods include DNA amplification using primers to introduce and amplify alterations in the DNA template, such as PCR splicing by overlap extension (SOE). Mutations may be introduced at a particular location by synthesizing oligonucleotides containing a mutant sequence, flanked by restriction sites enabling ligation to fragments of the native sequence. Following ligation, the resulting reconstructed sequence encodes a variant (or derivative) having the desired amino acid insertion, substitution, or deletion.

Site directed mutagenesis of a polynucleotide such that it encodes a CD47 extracellular domain variant may be performed according to any one of numerous methods described herein and practiced in the art (Kramer et al., Nucleic Acids Res. 12:9441 (1984); Kunkel Proc. Natl. Acad. Sci. USA 82:488-92 (1985); Kunkel et al., Methods Enzymol. 154:367-82 (1987)).

Random mutagenesis methods to identify residues that, when mutated (e.g., substituted or deleted), alter binding of the CD47 extracellular domain to a ligand, or that alter binding of the CD47 extracellular domain variant to a CD47-specific antibody, can also be performed according to procedures that are routinely practiced by a person skilled in the art (e.g., alanine scanning mutagenesis; error prone polymerase chain reaction mutagenesis; and oligonucleotide-directed mutagenesis (see, e.g., Sambrook et al. Molecular Cloning. A Laboratory Manual, 3rd ed., Cold Spring Harbor Laboratory Press, NY (2001)). In certain embodiments, a CD47 extracellular domain variant retains the capability to bind to at least one CD47 ligand (e.g., SIRP-α, SIRP-beta-2, thrombospondin-1, αᵥβ₃ integrin, and α₂β₁ integrin). In certain other embodiments, a CD47 fusion polypeptide comprising a CD47 extracellular domain variant retains the capability to bind to at least two, three, four, or five CD47 ligands. Further, as described herein, the CD47 extracellular domain variant and the fusion polypeptide comprising the variant retain at least one biological function, which are described herein, of the wild type CD47 moiety.

Site directed mutagenesis techniques may also be used to make a CD47 fusion polypeptide comprising a CD47 extracellular domain variant that exhibits an alteration (i.e., statistically or biologically significant increase or decrease) in the capability of the variant to bind specifically to a CD47 ligand when compared with the wildtype CD47 polypeptide. Such a CD47 extracellular domain variant may, for example, have at least one substitution, deletion, or addition of an amino acid such that the variant retains the capability to bind at least one CD47 ligand and exhibits a decreased (i.e., reduced, diminished) capability to bind specifically to at least one second CD47 ligand when compared with CD47 without the mutation. In other certain embodiments, the CD47 extracellular domain variant retains the capability to bind to at least two, three, or four, CD47 ligands and exhibits a reduced or decreased capability to bind to at least one CD47 ligand.

### CD47 Ligands

CD47 ligands include various polypeptides, such as signal regulatory proteins (SIRPs, such as SIRP-α, SIRP-beta-2); thrombospondin-1 and fragments and portions thereof such as the carboxy terminal portion of thrombospondin-1; and certain integrins, particularly β₃ and β₁ integrins, for example, αᵥβ₃ integrin and α₂β₁ integrin. CD47 ligands also include antibodies that specifically bind to CD47, particularly antibodies that bind to the extracellular domain of CD47.

As used herein, a CD47 fusion polypeptide is said to be “specific for” or to “specifically bind” a CD47 ligand if the CD47 extracellular domain moiety reacts at a detectable level with the ligand, preferably with an affinity constant, Kₐ, of greater than or equal to about 10⁴ M⁻¹, or greater than or equal to about 10⁵ M⁻¹, greater than or equal to about 10⁶ M⁻¹, greater than or equal to about 10⁷ M⁻¹, or greater than or equal to 10⁸ M⁻¹. The ability of the CD47 fusion polypeptide to bind to a CD47 ligand may also be expressed as a dissociation constant K_{D}, and a CD47 fusion polypeptide specifically binds to a CD47 ligand if it binds with a K_{D} of less than or equal to 10⁻⁴ M, less than or equal to about 10⁻⁵ M, less than or equal to about 10⁻⁶ M, less than or equal to 10⁻⁷ M, or less than or equal to 10⁻⁸ M.

Affinities of CD47, including the affinities of the extracellular domain of CD47, and the CD47 fusion polypeptides described herein, and its cognate ligands can be readily determined using conventional techniques, for example, those described by Scatchard et al. (Ann. N.Y. Acad. Sci. USA 51:660 (1949)) and by surface plasmon resonance (SPR; BIAcore™, Biosensor, Piscataway, N.J.). For surface plasmon resonance, target molecules are immobilized on a solid phase and exposed to ligands in a mobile phase running along a flow cell. If ligand binding to the immobilized target occurs, the local refractive index changes, leading to a change in SPR angle, which can be monitored in real time by detecting changes in the intensity of the reflected light. The rates of change of the surface plasmon resonance signal can be analyzed to yield apparent rate constants for the association and dissociation phases of the binding reaction. The ratio of these values gives the apparent equilibrium constant (affinity) (see, e.g., Wolff et al., Cancer Res. 53:2560-65 (1993)).

Binding of a fusion polypeptide (which also includes fusion polypeptide dimers) comprising a CD47 extracellular domain, or variant thereof, as described herein, to a CD47 ligand may prevent interaction between any one or more of the aforementioned CD47 ligands with CD47 expressed on the surface of a cell. Without wishing to be bound by theory, interaction between the fusion polypeptide and the CD47 ligand may alter a biological function of cell surface-expressed CD47 by preventing or inhibiting the cell surface CD47 from interacting with the CD47 ligand. In addition to, or alternatively, interaction between the fusion polypeptide and the CD47 ligand may initiate or stimulate a biological function of the CD47 ligand. For example, the CD47 fusion polypeptides described herein may alter the interaction, likely inhibit (i.e., prevent, diminish, reduce, decrease) binding of CD47 to β₃ and β₁ integrins, such as αᵥβ₃ integrin and αIIbβ₃, and α₂β₁ integrin, respectively, which have been described as CD47 ligands (see, e.g., Lindberg et al., J. Cell Biol. 123:485-96 (1993); Lindberg et al., J. Cell Biol. 134:1313-22 (1996); Wang et al., Mol. Biol. Cell 9:865-74 (1998); Wang et al., J. Cell Biol. 147:389-400 (1999); Brown et al., J. Cell Biol. 111:2785-94 (1990)). The integrin αᵥβ₃ is a cell receptor that is expressed by many cell types and that binds to a variety of different polypeptides via the RGD (arginine-glycine-aspartic acid) sequence (Brown et al., J. Cell Biol. 111:2785-94 (1990); Blystone et al., J. Cell Biol. 130:745-54 (1995)).

In another embodiment, a fusion polypeptide comprising a CD47 extracellular domain, or variant thereof, as described herein, may bind to one or more members of a family of transmembrane glycoproteins referred to as SIRPs (signal regulatory proteins) such as SIRPα and SIRP-beta-2. Interaction between the fusion polypeptide and a SIRP polypeptide may stimulate at least one biological function of a SIRP. For instance, the fusion polypeptides may alter the interaction between CD47 expressed on the surface of a cell and SIRPα (signal regulatory protein-α), which is also referred to in the art as SHPS-1 (Src homology 2 domain-containing protein tyrosine phosphatase-1 (SHP) substrate-1). SIRPs including SIRPα are expressed in hematopoietic cells including monocytes, granulocytes, dendritic cells, and CD34⁺CD38⁻CD133⁺ bone marrow stem cells, and has been reported to be expressed on smooth muscle cells (see, e.g., Latour et al., J. Immunol. 167:2547-54 (2001); Liu et al., J. Biol. Chem. 277:10028-36 (2002); Seiffert et al., Blood 94:3633-43 (1999); Seiffert et al., Blood 97:2741-49 (2001); Jiang et al. J. Biol. Chem. 274:559-62 (1999); Vernon-Wilson et al., Eur. J. Immunol. 30:2130-37 (2000); Oshima et al., FEBS Lett. 519:1-8 (2002)). SIRPα is believed to be an important immune inhibitor receptor on macrophages, and its interaction with CD47 prevents autologous phagocytosis (see, e.g., Oldenborg et al., J. Exp. Med. 193:855-62 (2001); Okazawa et al., J. Immunol. 174:2004-11 (2005)).

Interaction between a cell that expresses CD47 and a cell that expresses SIRPα can regulate cell migration, such as polymorphonuclear (PMN) cell transmigration and migration of Langerhans cells (see, e.g., Liu et al., supra; Motegi et al., EMBO J. 22:2634-44 (2003); Fukunaga et al., J. Immunol. 172:4091-99 (2004); Parkos et al., J. Cell Biol. 132:437-50 (1996)). A CD47-Fc fusion polypeptide described herein may inhibit migration of a cell that expresses SIRPα on the cell surface (i.e., the migrating cell) by inhibiting (i.e., preventing, blocking, or interfering with) the interaction between the migrating cell and a cell that has cellular CD47 present on the cell surface (such as an epithelial or endothelial cell).

Binding of a fusion polypeptide comprising a CD47 extracellular domain, or variant thereof, to SIRPα may alter the immunoresponsiveness of immune cells by inhibiting, decreasing, reducing, or preventing migration of immune cells and thus may inhibit, suppress, or decrease an inflammatory response in a host. SIRP polypeptides, such as SIRPα, are believed to act as negative regulatory cell receptors, such that, for example, stimulation of SIRP may inhibit Fc-mediated activation that may otherwise result in cytokine and/or chemokine production by an immune cell. Without wishing to be bound by theory, interaction between the CD47-Fc fusion polypeptides described herein and a SIRP polypeptide expressed by an immune cell induces a negative signal via SIRP signaling that may result in inhibition or decrease in an inflammatory response, such as production of cytokines, such as IL-6, IL-12, IL-23, TNF-α.

In another embodiment, binding of a fusion polypeptide comprising a CD47 extracellular domain, or variant thereof, to a CD47 ligand, may alter expression and/or secretion of cytokines, such as IL-12, IL-23, TNF-α, IFN-γ, IL-6, GM-CSF, and IL-10. Binding of a fusion polypeptide described herein to a CD47 ligand, such as SIRPα, may suppress release of cytokines such as IL-12, IL-23, TNF-α, IL-6, and IL-10. See, e.g., Latour et al., supra; Hermann et al., J. Cell Biol. 144:767-75 (1999); Armant et al., J. Exp. Med. 190:1175-81 (1999); Demeure et al., J. Immunol. 164:2193-99 (2000).

In another embodiment, a fusion polypeptide comprising a CD47 extracellular domain, or variant thereof, as described herein, may bind to thrombospondin-1, and in a particular embodiment may bind to the carboxy terminal portion of thrombospondin-1, also called Tp-47 in the art (see, e.g., Latour et al., supra). Thrombospondin-1 is an extracellular matrix protein that is released by platelets upon activation and is also produced by macrophages and monocytes. Binding of thrombospondin to cell-surface CD47 negatively regulates IL-12 production by antigen presenting cells (APC) and inhibits development of naïve T cells into Th1 effector cells (see, e.g., Armant et al., J. Exp. Med. 190:1175 (1999); Demeure et al., J. Immunol. 164:2193 (2000); Avice et al., J. Immunol. 165:4624 (2000)). The cytokine IL-12 can act as an inflammatory mediator, and uncontrolled IL-12 production and responsiveness are associated with certain immunological diseases, such as autoimmune diseases. Binding of the fusion polypeptides described herein to thrombospondin-1 may alter immunoresponsiveness of an immune cell by down regulating or facilitating decreased expression of IL-12.

Interaction between thrombospondin-1 and CD47 expressed on the cell surface also may induce apoptosis of activated T cells, which may be mediated by BNIP3 (Bcl-2 homology 3 (BH3)-only protein 19 kDa interacting protein-3) (Lamy et al., J. Biol. Chem. 278 (23915-21 (2003)). A fusion polypeptide comprising a CD47 extracellular domain, or variant thereof, as described herein, may inhibit binding of thrombospondin-1 to CD47 expressed by T cells, thereby altering apoptosis in lymphocytes. Accordingly, the fusion polypeptides described herein may be used for treating proliferative diseases, such as cancer.

The term IL-12 refers to the cytokine IL-12 produced by immune cells, including dendritic cells, and includes IL-12 related monomers and dimers that are described in the art. A bioactive form of IL-12 is a heterodimer called IL12p70 that is comprised of independently regulated subunits called p40 (IL12p40) and p35 (IL12p35) having an approximate molecular weight of 40 kilodaltons and 35 kilodaltons, respectively. IL12p40 may also exists as a dimer (IL12(p40)₂). See, for example, Gately et al., Annu. Rev. Immunol. 16:495 (1998); Hildens et al., Blood 90:1920 (1997); Kalinski et al., J. Immunol. 165:1877-81 (2000). IL-23 also comprises the p40 subunit of IL12 and is produced, for example, by dendritic cells and may act on memory T cells (see, e.g., Oppmann et al., Immunity 13:715-25 (2000); Wiekowski et al., J. Immunol. 166:7563-7570 (2001); Aggarwal, et al., J. Biol. Chem. 17:278:1910-14 (2003), Epub 2002 Nov. 3). The effect of IL-23 production and inflammatory diseases has recently led to the observation that expression of a receptor for IL-23 is associated with inflammatory diseases such as Crohn's disease and ulcerative colitis (see, e.g., Duerr et al., Science 314:1461-63 (2006); see also, e.g., Mannon et al., N. Engl. J. Med. 351:2069 (2004); Becker et al., J. Immunol. 177:2760-64 (2006); Lankford et al., J. Leukoc. Biol. 73:49-56 (2003)).

The CD47-Fc polypeptide fusion proteins described herein may also affect the capability of an immune complex to induce production of cytokines by an immune cell; thus the fusion proteins described herein may inhibit induction by an immune complex of cytokine production in an immune cell (i.e., inhibit immune complex-induced cytokine production by an immune cell). An immune cell includes but is not limited to an immune cell that expresses a CD47 ligand, for example, that expresses SIRPα. As described herein, such immune cells include, for example, a dendritic cell, a monocyte, macrophage, granulocyte, and a bone-derived stem cell. Thus, in certain specific embodiments, the CD47-Fc polypeptide fusion proteins described herein are capable of altering the immunoresponsiveness of a dendritic cell. Interaction of a CD47-Fc polypeptide fusion protein with an immune cell, for example, a dendritic cell, or with a ligand or other cell that interacts with the immune cell, such as the dendritic cell, alters (generally inhibits) the production of cytokines, including but not limited to IL-6, IL-12, IL-23, and TNF-α, by the dendritic cell. In other certain embodiments, the CD47-Fc polypeptide fusion proteins described herein alter, and in certain particular embodiments, inhibit or decrease, the capability of an immune complex to induce production of cytokines by an immune cell. In particular embodiments, the CD47-Fc fusion polypeptides described herein inhibit immune complex-induced cytokine production by an immune cell, such as a dendritic cell. Thus, and as discussed herein, the CD47-Fc polypeptide fusion proteins may be used for treatment of immunological diseases or disorders, such as autoimmune diseases (by way of example, arthritis). In addition, a CD47-Fc polypeptide fusion protein may inhibit or prevent maturation of a dendritic cell. The CD47 Fc fusion polypeptides described herein may also interact with SIRPα present on the cell surface of a neuronal, and consequently affect signaling of a neuronal cell function.

### Viral CD47 Polypeptides and Extracellular Domain Fragments Thereof

In another embodiment, the CD47 extracellular domain is a viral CD47-like polypeptide, for example, a poxvirus CD47-like polypeptide such as a variola minor poxvirus CD47-like polypeptide (see, e.g., SEQ ID NOS: 3 and 4). By encoding proteins that are viral homologues of cell cytokines and chemokines and their receptors, members of the poxvirus family have evolved numerous mechanisms to evade detection and elimination by an infected host's immune system. One viral virulence factor includes a polypeptide that shares structural homology with CD47. The viral CD47 polypeptides (also called viral CD47-like polypeptides or vCD47) have amino acid sequences that share 30% or less primary sequence homology; however, comparison of structural features of viral CD47 to immunomodulatory polypeptides indicates that the viral CD47 polypeptides have an N-terminal signal sequence, five to six transmembrane domains, and a short intracellular (i.e., cytoplasmic) tail, similar to CD47 (see, e.g., Cameron et al., Virology 264:298-318 (1999); Cameron et al., Virology 337:55-67 (2005); Alfonso et al., J. Virol. 79:966-77 (2005); Seet et al., Annu. Rev. Immunol. 21:377-423 (2003)).

Viral genomic sequences that encode viral CD47-like polypeptides have been identified in several poxviruses, including myxoma and orthopoxviruses as well as chordopoxvirus, a capripoxvirus, a leporipoxvirus, a suipoxvirus, a yatapoxvirus, and a deerpox virus. Exemplary amino acid sequences of viral CD47-like polypeptides include the A44L polypeptide encoded by the genome of Variola minor virus (GenBank Accession No. CAB54747.1 (SEQ ID NO:3)); Vaccinia virus-Western Reserve (GenBank Accession No. AA089441.1); Vaccinia virus (Acambis 3000 modified virus Ankara) (GenBank Accession No. AAT10547.1); Vaccinia virus (GenBank Accession No. YP_{—}233044.1); and the M128L polypeptide encoded by the genome of the Lausanne strain of Myxoma virus (GenBank Accession Nos. NP_{—}051842.1 and AAF15016.1). By way of example, an alignment of the amino acid sequences of the A44L polypeptide and a human CD47 isoform (see FIG. 2) indicates that the primary sequences of the two polypeptides are approximately 25% identical and approximately 60% similar.

In certain embodiments, a fusion polypeptide is provided herein that comprises a viral CD47 (vCD47) extracellular domain fused to a fused to a moiety capable of multimer formation (e.g., dimer formation), including for example, an Fc polypeptide and variants thereof. In one embodiment, the viral CD47 extracellular domain moiety is derived from the A44L amino acid sequence and comprises 105 amino acids (SEQ ID NO:4), which lacks the 20-amino acid signal peptide sequence (amino acids at positions 1-20 of SEQ ID NO:3) (see also FIG. 2). In certain other embodiments, a fusion polypeptide comprises a viral CD47 extracellular domain derived from another poxvirus CD47-like polypeptide described herein or known in the art. Persons skilled in the art can readily determine the portion of a viral CD47-like polypeptide that comprises the signal peptide, extracellular domain, transmembrane domains, and intracellular domains using alignment methods and other methods, such as von Heijne transmembrane plots described herein and used in the art

In other embodiments, a vCD47 fusion polypeptide comprises a vCD47 extracellular domain variant. Such a vCD47 fusion polypeptide retains the ability to bind specifically to at least one CD47 ligand. Such ligands are described herein and include SIRP-α, SIRP-beta 2, thrombospondin-1, αᵥβ₃ integrin, and α₂β₁ integrin, for example. The vCD47 fusion polypeptide that comprises a vCD47 variant also retains the capability to competitively inhibit binding of CD47 to at least one CD47 ligand. The vCD47 extracellular domain variant may have an amino, acid sequence that is at least 65%-75%, 75%-80%, 80-85%, 85%-90%, or 95%-99% identical (which includes any percent identity between any one of the described ranges) to the amino acid sequence of the corresponding wildtype poxvirus CD47-like polypeptide, such as a Variola minor vCD47 extracellular domain as set forth in SEQ ID NO:4. Such vCD47 extracellular domain variants may retain cysteine residues that form the intramolecular disulfide bond, which confers an immunoglobulin domain-like structure. By way of example, the cysteine residues in the Variola minor vCD47 (A44L) amino acid sequence that typically form an intramolecular disulfide bond are located at positions that correspond to the cysteine residues at position 16 and position 79 of SEQ ID NO:4. Persons skilled in the art will appreciate that the cysteine residues that typically form an intramolecular disulfide bond in one vCD47 may be located at positions that differ from the numbered positions of the cysteine residues in SEQ ID NO:4 and that determining the location of corresponding cysteine residues in another amino acid sequence that form an intramolecular disulfide bond in a vCD47 polypeptide is well within the routine practice of the skilled artisan using alignment programs and molecular modeling programs described herein and used in the art.

The percent identity of a vCD47 extracellular domain variant compared with the wildtype vCD47 amino acid sequence can be readily determined by persons skilled in the art by sequence comparison. As used herein, two amino acid sequences have 100% amino acid sequence identity if the amino acid residues of the two amino acid sequences are the same when aligned for maximal correspondence. Sequence comparisons of polypeptides and polynucleotides can be performed using any method including using computer algorithms well known to persons having ordinary skill in the art, and include algorithms described herein for alignment of CD47 polypeptides and the polynucleotides that encode the CD47 polypeptides.

A vCD47 extracellular domain variant may differ from a wildtype vCD47 amino acid sequence (e.g., the amino acid sequence set forth in SEQ ID NO:4) due to an insertion, deletion, addition, and/or substitution of at least one amino acid and may differ due to the insertion, deletion, addition, and/or substitution of at least two, three, four, five, six, seven, eight, nine, or ten amino acids or may differ by any number of amino acids between 10 and 45 amino acids. A vCD47 extracellular domain variant includes, for example, a naturally occurring polymorphism that occurs due to a mutation introduced into the viral genome during infection and/or replication in a host or a recombinantly manipulated or engineered vCD47 extracellular domain variant.

A vCD47 extracellular domain variant that differs from the amino acid sequence set forth in SEQ ID NO:4, for example, includes a variant with at least one deletion from either the amino terminal end or carboxy terminal end or from both the amino terminal end and the carboxy terminal end of the vCD47 extracellular domain. Such a vCD47 extracellular domain variant may also be referred to herein as a truncated vCD47 extracellular domain. The truncation may include a deletion of at least 1, 2, 3, 4, 5, 6, 7, 8, 9, 10, 12, or more amino acids from the amino terminal end or carboxy terminal end of the CD47 extracellular domain or may include a deletion of between 1-20 amino acids from each terminal end. In particular embodiments, a truncated vCD47 extracellular domain variant retains the cysteine residues that form the intramolecular disulfide bond.

A vCD47 fusion polypeptide that comprises a vCD47 extracellular domain variant that retains the capability to bind to at least one CD47 ligand and/or the capability to competitively inhibit binding of CD47 to a CD47 ligand includes variants that contain conservative amino acid substitutions. A variety of criteria known to persons skilled in the art indicate whether an amino acid that is substituted at a particular position in a peptide or polypeptide is conservative (or similar). For example, a similar amino acid or a conservative amino acid substitution is one in which an amino acid residue is replaced with an amino acid residue having a similar side chain. Similar amino acids may be included in the following categories: amino acids with basic side chains (e.g., lysine, arginine, histidine); amino acids with acidic side chains (e.g., aspartic acid, glutamic acid); amino acids with uncharged polar side chains (e.g., glycine, asparagine, glutamine, serine, threonine, tyrosine, cysteine, histidine); amino acids with nonpolar side chains (e.g., alanine, valine, leucine, isoleucine, proline, phenylalanine, methionine, tryptophan); amino acids with beta-branched side chains (e.g., threonine, valine, isoleucine), and amino acids with aromatic side chains (e.g., tyrosine, phenylalanine, tryptophan). Proline, which is considered more difficult to classify, shares properties with amino acids that have aliphatic side chains (e.g., leucine, valine, isoleucine, and alanine). In certain circumstances, substitution of glutamine for glutamic acid or asparagine for aspartic acid may be considered a similar substitution in that glutamine and asparagine are amide derivatives of glutamic acid and aspartic acid, respectively. As understood in the art “similarity” between two polypeptides is determined by comparing the amino acid sequence and conserved amino acid substitutes thereto of the polypeptide to the sequence of a second polypeptide (e.g., using GENEWORKS, Align, the BLAST algorithm, or other algorithms described herein and practiced in the art).

In certain embodiments, a fusion polypeptide comprising a vCD47 extracellular domain, or a variant thereof, is recombinantly expressed. For instance, a vCD47 extracellular domain, or variant thereof, is fused in frame with a Fc polypeptide, or variant thereof, as described in detail herein. A recombinant expression construct may be prepared for the expression of a vCD47 fusion polypeptide according to standard techniques and methods practiced by a skilled person in the molecular biology art. In order to obtain efficient transcription and translation, the polynucleotide sequence in each construct should include appropriate regulatory sequences, particularly a promoter and leader sequence operatively linked to a nucleotide sequence encoding the vCD47 extracellular domain, or variant thereof, or the promoter may be operatively linked to the nucleotide sequence encoding the signal peptide sequence located at the amino terminal end of the vCD47 extracellular domain. Particular methods for producing polypeptides recombinantly are generally well known and routinely used. For example, molecular biology procedures are described by Sambrook et al. (Molecular Cloning, A Laboratory Manual, 2nd ed., Cold Spring Harbor Laboratory, New York, 1989; see also Sambrook et al., 3rd ed., Cold Spring Harbor Laboratory, New York, (2001)). DNA sequencing can be performed as described in Sanger et al. (Proc. Natl. Acad. Sci. USA 74:5463 (1977)) and the Amersham International plc sequencing handbook and including improvements thereto.

Exemplary nucleotide sequences that encode the vCD47 extracellular domain portion of a vCD47 fusion polypeptide are provided herein and are readily available from public databases that provide the genomic sequences of various poxviruses, including, for example, Myxoma virus (GenBank Accession No. AF170726.2); Variola minor virus (GenBank Accession No. Y16780.1); Yaba monkey tumor virus (GenBank Accession Nos. NC_{—}005179.1; AY386371.1); Yaba-like disease virus (GenBank Accession Nos. AJ293568.1; NC_{—}002642.1); mule deerpox virus (NC_{—}006966.1).

The nucleotide sequence of a vCD47 extracellular domain variant can be determined and/or identified by comparing the nucleotide sequence of a polynucleotide encoding the variant with a polynucleotide described herein or known in the art that encodes a vCD47 polypeptide using any one of the alignment algorithms described herein and used in the art. The percent identity between two polynucleotides may thus be readily determined. Polynucleotides have 100% nucleotide sequence identity if the nucleotide residues of the two sequences are the same when aligned for maximal correspondence. In particular embodiments, the nucleotide sequence of a vCD47 extracellular domain variant-encoding polynucleotide is at least 70%, 75%, 80%, 85%, 90%, 95%, or 98% identical to one or more of the polynucleotide sequences that encode a wild type vCD47 extracellular domain, which are described herein. Polynucleotide variants also include polynucleotides that differ in nucleotide sequence identity due to the degeneracy of the genetic code but encode a vCD47 extracellular domain having an amino acid sequence disclosed herein or known in the art. A polynucleotide that encodes a vCD47 fusion polypeptide as described herein also includes a polynucleotide that is complementary to such a polynucleotide. Certain polynucleotides that encode a vCD47 extracellular domain, variant, or fragment thereof may also be used as probes and primers. Polynucleotides may be single-stranded DNA or RNA (coding or antisense) or double-stranded RNA (e.g., genomic or synthetic) or DNA (e.g., cDNA or synthetic). Polynucleotide variants may be identified by alignment procedures described herein and also may be identified by hybridization methods as described herein for identifying and characterizing polynucleotide variants that encode CD47.

vCD47 extracellular domain variants may be readily prepared by genetic engineering and recombinant molecular biology methods and techniques. Analysis of the primary and secondary amino acid sequence of a vCD47 polypeptide and of the vCD47 extracellular domain and computer modeling of same to analyze the tertiary structure of the polypeptide may aid in identifying specific amino acid residues that can be substituted, added, or deleted without altering the structure and as a consequence, potentially the function, of the vCD47 polypeptide. Modification of DNA encoding a vCD47 extracellular domain variant may be performed by a variety of methods, including site-specific or site-directed mutagenesis of the DNA, which methods include DNA amplification using primers to introduce and amplify alterations in the DNA template, such as PCR splicing by overlap extension (SOE). Mutations may be introduced at a particular location by synthesizing oligonucleotides containing a mutant sequence, flanked by restriction sites enabling ligation to fragments of the native sequence. Following ligation, the resulting reconstructed sequence encodes a variant (or derivative) having the desired amino acid insertion, substitution, or deletion.

Site directed mutagenesis of a polynucleotide to encode a vCD47 extracellular domain variant may be performed according to any one of numerous methods described herein and practiced in the art (Kramer et al., Nucleic Acids Res. 12:9441 (1984); Kunkel Proc. Natl. Acad. Sci. USA 82:488-92 (1985); Kunkel et al., Methods Enzymol. 154:367-82 (1987)). Random mutagenesis methods to identify residues that, when mutated (e.g., substituted or deleted), alter the binding affinity of the vCD47 extracellular domain to a CD47 ligand or that alter the capability the vCD47 extracellular domain variant to competitively inhibit binding of CD47 to a CD47 ligand can also be performed according to procedures that are routinely practiced by a person skilled in the art (e.g., alanine scanning mutagenesis; error prone polymerase chain reaction mutagenesis; and oligonucleotide-directed mutagenesis (see, e.g., Sambrook et al. Molecular Cloning. A Laboratory Manual, 3rd ed., Cold Spring Harbor Laboratory Press, NY (2001)). In certain embodiments, a vCD47 extracellular domain variant retains the capability to bind to at least one CD47 ligand (e.g., SIRP-α, SIRP-beta-2, thrombospondin-1, αᵥβ₃ integrin, and α₂β₁ integrin). In certain other embodiments, a vCD47 fusion polypeptide comprising a vCD47 extracellular domain variant that retains the capability to bind to at least two, three, four, or five, CD47 ligands.

Site directed mutagenesis techniques may also be used to make a fusion polypeptide comprising a vCD47 extracellular domain variant that exhibits an alteration (i.e., statistically or biologically significant increase or decrease) in the capability of the variant to bind specifically to a CD47 ligand when compared with the wildtype vCD47 polypeptide. Such a vCD47 extracellular domain variant may, for example, have at least one substitution, deletion, or addition of an amino acid such that the variant retains the capability to bind at least one CD47 ligand and exhibits a decreased (i.e., reduced, diminished) capability to bind specifically to at least one second CD47 ligand when compared with vCD47 without the mutation. In other certain embodiments, the vCD47 extracellular domain variant retains the capability to bind to at least two, three, or four, CD47 ligands and exhibits a reduced or decreased capability to bind to at least one CD47 ligand.

In another embodiment, a fusion polypeptide comprises a vCD47 extracellular domain variant fused to a Fc polypeptide (or variant thereof), wherein the vCD47 extracellular domain variant comprises an amino acid sequence at least 65%-75%, 75%-80%, 80-85%, 85%-90%, or 95%-99% identical to SEQ ID NO:4 and retains the capability to bind at least one CD47 ligand (e.g., SIRP-α, SIRP-beta-2, thrombospondin-1, αᵥβ₃ integrin, and α₂β₁ integrin). In another particular embodiment, the vCD47 extracellular domain variant comprises a substitution or a deletion of the cysteine residue that is most proximal to the amino terminal end of the vCD47 extracellular domain. In certain embodiments, this cysteine residue is located at a position corresponding to the cysteine residue at position 8 of SEQ ID NO:4. In a particular embodiment, the cysteine residue may be substituted with any amino acid, for example the cysteine residue may be substituted with a serine residue. In another particular embodiment, the cysteine residue may be substituted with one, two, or three, or four amino acids. For example, the cysteine residue most proximal to the amino terminal end of CD47, which corresponds to the cysteine at position 8 of SEQ ID NO:4, may be substituted with a tripeptide that is a potential glycosylation site, for example, a tripeptide that has the sequence Asn-X-Ser wherein X may be any amino acid except cysteine. If desired, substitution or deletion of a cysteine residue that corresponds to the cysteine at position 8 of SEQ ID NO:4 abrogates the possibility that a vCD47 extracellular domain moiety of one fusion polypeptide will form a dimer with another vCD47 extracellular domain moiety via formation of a disulfide bond between the cysteine residues that are located most proximal to the amino terminal end of the vCD47 extracellular domain amino acid sequence.

A fusion polypeptide comprising a vCD47 extracellular domain, or a variant thereof, may be used in competition binding assays to identify a binding site of CD47 that interacts with a CD47 ligand. The fusion polypeptide comprising a vCD47 extracellular domain, or a variant thereof, may be also used to alter (i.e., increase or decrease in a statistically significant or biologically significant manner) immunoresponsiveness of an immune cell. Such a fusion polypeptide may therefore also be used to alter the immune response of a subject and thereby may be useful for treating an immunological disease or disorder.

In one embodiment, a fusion polypeptide comprising vCD47 extracellular domain or a variant thereof as described herein may be used for treating a patient who presents an acute immune response. For example, a fusion polypeptide comprising vCD47 extracellular domain or a variant thereof may suppress an immune response associated with a disease or condition such as acute respiratory distress syndrome (ARDS). ARDS, which may develop in adults and in children, often follows a direct pulmonary or systemic insult (for example, sepsis, pneumonia, aspiration) that injures the alveolar-capillary unit. Several cytokines are associated with development of the syndrome, including, for example, tumor necrosis factor-alpha (TNF-α), interleukin-beta (IL-β), IL-10, and soluble intercellular adhesion molecule 1 (sICAM-1). The increased or decreased level of these factors and cytokines in a biological sample may be readily determined by methods and assays described herein and practiced routinely in the art to monitor the acute state and to monitor the effect of treatment.

To reduce or minimize the possibility or the extent of an immune response that is specific for vCD47, or fragment thereof, the fusion polypeptide may be administered in a limited number of doses, may be produced or derived in a manner that alters glycosylation of vCD47, and/or may be administered under conditions that reduce or minimize antigenicity of vCD47. For example, a fusion polypeptide comprising vCD47 extracellular domain or a variant thereof may be administered prior to, concurrently with, or subsequent to the administration in the host of a second composition that suppresses an immune response, particularly a response that is specific for vCD47.

### Use of Fusion Polypetides Comprising a CD47 Extracellular Domain, or Variant Thereof, for Altering Immunoresponsiveness of an Immune Cell

In one embodiment, any one of the fusion polypeptides comprising a CD47 extracellular domain, or variant thereof, or any one of the fusion polypeptides comprising a vCD47 extracellular domain as described herein may be used to alter (enhance or suppress in a statistically significant or biologically significant manner) the immunoresponsiveness of an immune cell. Any one of the fusion polypeptides described herein may alter or affect the immunoresponsiveness of an immune cell by effecting a biological function or action, including any one or more (or at least one of) the following: inhibiting maturation of dendritic cells; impairing development of naïve T cells into Th1 effector cells; suppressing cytokine release by dendritic cells; altering cell migration; inhibiting production of at least one cytokine, for example, at least one of TNF-α, IL-12, IL-23, IFN-γ, GM-CSF, and IL-6; inhibiting immune complex-induced production of at least one cytokine by an immune cell, such, for example, a dendritic cell; inhibiting activation of an immune cell that expresses a CCD47 ligand, for example SIRP-alpha, inhibiting production of a chemokine by an immune cell; inhibiting Fc-mediated cytokine production; and suppressing a proinflammatory response.

An immune cell is any cell of the immune system, including a lymphocyte and a non-lymphoid cell such as accessory cell. Lymphocytes are cells that specifically recognize and respond to foreign antigens, and accessory cells are those that are not specific for certain antigens but are involved in the cognitive and activation phases of immune responses. For example, mononuclear phagocytes (macrophages), other leukocytes (e.g., granulocytes, including neutrophils, eosinophils, basophils), and dendritic cells function as accessory cells in the induction of an immune response. Immune cells include cells that express a CD47 ligand. For example, an immune cell includes a cell that expresses SIRP-α, which includes a monocyte, macrophage, dendritic cell, granulocyte, and a CD34⁺CD38⁻CD133⁺ bone marrow stem/progenitor cell (see, g., Seiffert et al., Blood 94:3633 (1999); Seiffert et al., Blood 97:2741 (2001)). The activation of lymphocytes by a foreign antigen leads to induction or elicitation of numerous effector mechanisms that function to eliminate the antigen. Accessory cells such as mononuclear phagocytes that effect or are involved with the effector mechanisms are also called effector cells.

Major classes of lymphocytes include B lymphocytes (B cells), T lymphocytes (T cells), and natural killer (NK) cells, which are large granular lymphocytes. B cells are capable of producing antibodies. T lymphocytes are further subdivided into helper T cells (CD4+) and cytolytic or cytotoxic T cells (CD8+). Helper cells secrete cytokines that promote proliferation and differentiation of the T cells and other cells, including B cells and macrophages, and recruit and activate inflammatory leukocytes. Another subgroup of T cells, called regulatory T cells or suppressor T cells actively suppress activation of the immune system and prevent pathological self-reactivity, that is, autoimmune disease.

In general, an immune response includes (1) a humoral response, in which antibodies specific for antigens are produced by differentiated B lymphocytes known as plasma cells, and (2) a cell mediated response, in which various types of T lymphocytes act to eliminate antigens by a number of mechanisms. For example, helper T cells that are capable of recognizing specific antigens may respond by releasing soluble mediators such as cytokines to recruit additional cells of the immune system to participate in an immune response. Also, cytotoxic T cells that are also capable of specific antigen recognition may respond by binding to and destroying or damaging an antigen-bearing cell or particle.

An immune response in a host or subject may be determined by any number of well-known immunological methods described herein and with which those having ordinary skill in the art will be readily familiar. Such assays include, but need not be limited to, in vivo or in vitro determination of soluble antibodies, soluble mediators such as cytokines (e.g., IFN-γ, IL-2, IL-4, IL-10, IL-12, IL-6, IL-23, TNF-α, and TGF-β), lymphokines, chemokines, hormones, growth factors, and the like, as well as other soluble small peptide, carbohydrate, nucleotide and/or lipid mediators; cellular activation state changes as determined by altered functional or structural properties of cells of the immune system, for example cell proliferation, altered motility, induction of specialized activities such as specific gene expression or cytolytic behavior; cell maturation, such as maturation of dendritic cells in response to a stimulus; alteration in relationship between a Th1 response and a Th2 response; cellular differentiation by cells of the immune system, including altered surface antigen expression profiles or the onset of apoptosis (programmed cell death). Procedures for performing these and similar assays are may be found, for example, in Lefkovits (Immunology Methods Manual. The Comprehensive Sourcebook of Techniques, 1998). See also Current Protocols in Immunology; Weir, Handbook of Experimental Immunology, Blackwell Scientific, Boston, Mass. (1986); Mishell and Shigii (eds.) Selected Methods in Cellular Immunology, Freeman Publishing, San Francisco, Calif. (1979); Green and Reed, Science 281:1309 (1998) and references cited therein).

Levels of cytokines may be determined according to methods described herein and practiced in the art, including ELISA, ELISPOT, and flow cytometry (to measure intracellular cytokines). Immune cell proliferation and clonal expansion resulting from an antigen-specific elicitation or stimulation of an immune response may be determined by isolating lymphocytes, such as spleen cells or cells from lymph nodes, stimulating the cells with antigen, and measuring cytokine production, cell proliferation and/or cell viability, such as by incorporation of tritiated thymidine or non-radioactive assays, such as MTT assays and the like. The effect of a fusion polypeptide described herein on the balance between a Th1 immune response and a Th2 immune response may be examined, for example, by determining levels of Th1 cytokines, such as IFN-γ, IL-12, IL-2, and TNF-β, and Type 2 cytokines, such as IL-4, IL-5, IL-9, IL-10, and IL-13.

Methods and techniques for determining the effect of a fusion polypeptide comprising a CD47 extracellular domain, or variant thereof, (or viral CD47 extracellular domain or variant thereof) may also be found in Armant et al., J. Exp. Med. 190:1175 (1999); Demeure et al., J. Immunol. 164:2193 (2000); Avice et al., J. Immunol. 165:4624 (2000); Lamy et al., J. Biol. Chem. 278 (23915-21 (2003); Hermann et al., J. Cell Biol. 144:767-75 (1999); Liu et al., J. Biol. Chem. 277:10028-36 (2002); Seiffert et al., Blood 94:3633-43 (1999); Seiffert et al., Blood 97:2741-49 (2001); Motegi et al., EMBO J. 22:2634-44 (2003); Fukunaga et al., J. Immunol. 172:4091-99 (2004); Parkos et al., J. Cell Biol. 132:437-50 (1996); and International Application Publication Nos. WO 99/40940 and WO 97/27873.

In certain embodiments, a fusion polypeptide described herein that comprises a CD47 extracellular domain or variant thereof exhibits the capability to competitively inhibit binding of at least one CD47 ligand to CD47 expressed by a cell and located at the cell surface. The CD47 ligand may be at least one of SIRP-α, SIRP-beta-2, thrombospondin-1, αᵥβ₃ integrin, and α₂β₁ integrin. In certain other embodiments, such a fusion polypeptide also has the capability to competitively inhibit binding of viral CD47 polypeptide to at least one CD47 ligand. The viral CD47 polypeptide includes an isolated full-length viral CD47 polypeptide, a viral CD47 extracellular domain, or viral CD47 expressed on the surface of a cell. In certain embodiments, the fusion polypeptide competitively inhibits binding of an at least one CD47 ligand to cell surface-expressed CD47 and competitively inhibits binding of the same ligand to viral CD47. In other embodiments, the at least one, two, three, four, or five, or more CD47 ligands that are competitively inhibited from binding to a cell-surface expressed CD47 in the presence of a fusion polypeptide comprising CD47 extracellular domain or variant thereof may not be the same one, two, three, four, or five, or more CD47 ligands that are competitively inhibited from binding to viral CD47. The viral CD47 polypeptide includes an isolated full-length viral CD47 polypeptide, a viral CD47 extracellular domain, or viral CD47 expressed on the surface of a cell. Examples of viral CD47 polypeptides are described herein and can be readily identified in the relevant art by a skilled artisan. Any of a number of assays described herein and practiced in the art for determining the level of binding between a fusion polypeptide comprising a CD47 extracellular domain or variant thereof and a CD47 ligand can be modified to a format for determining the capability of the fusion polypeptide to bind to the ligand in the presence of a viral CD47 polypeptide. Such modifications are routinely performed by persons skilled in the art.

### Fc Polypeptide Moiety of the Fusion Polypeptides

In one embodiment, a fusion polypeptide comprising a CD47 extracellular domain, or a variant thereof, is fused to an Fc polypeptide. An Fc polypeptide, which includes a mutein Fc polypeptide (that is, an Fc polypeptide into which a substitution, deletion, or insertion of at least one amino acid has been introduced, also called a Fc polypeptide variant), is derived from the constant region portion of an immunoglobulin. An Fc polypeptide comprises the heavy chain CH2 domain, the CH3 domain, and a portion of, or the entire, hinge region that is located between the heavy chain CH1 domain and CH2.

Historically, the Fc fragment was derived by papain digestion of an immunoglobulin and included the hinge region of the immunoglobulin. Fc regions are monomeric polypeptides that may be linked into dimeric or multimeric forms by covalent (e.g., particularly disulfide bonds) and non-covalent association. The number of cysteine residues in the hinge portion of an Fc polypeptide varies depending on the immunoglobulin class (e.g., IgG, IgA, IgE) or subclass (e.g., human IgG1, IgG2, IgG3, IgG4, IgA1, IgA2), and thus the number of intermolecular disulfide bonds that form between the hinge portions of monomeric subunits of Fc polypeptides varies.

In one embodiment, the Fc polypeptide is of human origin and may be from any of the immunoglobulin classes, such IgG or IgA, and from any subclass such as human IgG1, IgG2, IgG3, and IgG4. In a certain embodiment, the Fc polypeptide is derived from a human IgG1 immunoglobulin. In another embodiment, the CD47 extracellular domain-Fc fusion polypeptide comprises an Fc polypeptide from a non-human animal, for example, but not limited to, a mouse, rat, rabbit, camel, shark, non-human primate, or hamster. The amino acid sequence of an Fc polypeptide derived from an immunoglobulin of the same host species to which an CD47 extracellular domain-Fc fusion polypeptide may be administered is likely to be non-immunogenic, or less immunogenic, than an Fc polypeptide from a non-syngeneic host. In certain other embodiments, a particular property attributed to an Fc polypeptide of a non-syngeneic species (for example, a Fc polypeptide from a non-human species fused to a human CD47 extracellular domain for administration to a human subject) may be desirable. Such an Fc polypeptide may be altered, such as by introducing amino acid substitutions or in some manner altering the glycosylation pattern, to reduce the immunogenicity of the Fc polypeptide when introduced into a non-syngeneic host. As described herein, immunoglobulin sequences of a variety of species are available in the art, for example, in Kabat et al. (in Sequences of Proteins of Immunological Interest, 4th ed (U.S. Dept. of Health and Human Services, U.S. Government Printing Office, 1991)). A person skilled in the molecular biology art can readily prepare such fusion polypeptides according to methods described herein and practiced routinely in the art.

In one embodiment, a fusion polypeptide comprising a CD47 (e.g., a human CD47) extracellular domain fused to a Fc polypeptide comprises the amino acid sequence set forth in SEQ ID NO:2. In other specific embodiments, the amino acid sequence of the fusion polypeptide comprises an amino acid sequence that is at least 65%-75%, 75%-80%, 80-85%, 85%-90%, or 95%-99% identical (which includes any percent identity between any one of the described ranges) to SEQ ID NO:2. As discussed herein, the CD47 extracellular domain moiety of the fusion polypeptide may contain at least one amino acid substitution, deletion, or insertion compared with a wildtype CD47 sequence. In addition, or instead, the Fc polypeptide moiety may comprises at least one amino acid substitution, deletion, or insertion compared with a wildtype Fc amino acid sequence. Furthermore, as described in Kabat et al., the Fc polypeptides of all immunoglobulins, while conserved, are not necessarily identical. Therefore, natural Fc polypeptides (that is, those identified as being produced in a human or non-human animal) are not necessarily identical, and for example, may be at least 85%, 90%, or 95% identical to the amino acid sequence set forth in any of the sequences disclosed herein and known in the art but which may readily be used for making a fusion polypeptide described herein.

In certain embodiments, the Fc polypeptide is a mutein Fc polypeptide (also called an Fc polypeptide variant herein), which has a substitution, deletion, or addition of at least 1, 2, 3, 4, 5, 6, 7, 8, 9, 10, or 10-15, 16-25 amino acids. In one embodiment, the mutein Fc polypeptide has at least 1, 2, 3, or more amino acid substitutions, deletions, or additions in the hinge region of the Fc polypeptide. In another embodiment, the mutein Fc polypeptide has at least 1, 2, 3, 4, 5, 6, 7, 8, 9, 10 or more amino acid substitutions, deletions, or additions in the CH2 domain and/or in the CH3 domain of the Fc polypeptide. A mutein Fc polypeptide also includes fragments of an Fc polypeptide, such as an Fc polypeptide that is truncated at the C-terminal end (that is at least 1, 2, 3, 4, 5, 10, 15, 20, or more amino acids have been removed or deleted).

In certain embodiments, the Fc polypeptides described herein contain multiple cysteine residues, such as at least some or all of the cysteine residues in the hinge region, to permit interchain disulfide bonds to form between the Fc polypeptide portions of two separate fusion proteins, such that two CD47 extracellular domain (or variant thereof)/Fc polypeptides fusion proteins form dimers through interaction between the Fc portions of the fusion polypeptide. In other embodiments, the Fc polypeptide comprises substitutions or deletions of cysteine residues in the hinge region such that an Fc polypeptide fusion protein is monomeric and fails to form a dimer (see, e.g., U.S. Patent Application Publication No. 2005/0175614).

An Fc polypeptide may be fused to a CD47 extracellular domain via covalent attachment such as by conjugation procedures practiced in the art for covalently linking two separate amino acid-containing moieties, for example, using maleimide or carbidiimide coupling chemistry (see also, e.g., Carlsson et al., Biochem. J. 173:723-37 (1978)). The site of conjugation can be at either the amino terminus or the carboxy terminus or in the middle of the sequence. The point of conjugation may be a sulfhydryl (SH) group or an amino group (NH₂).

An Fc polypeptide, and any one or more constant region domains, and fusion proteins comprising at least one immunoglobulin constant region domain may also be readily prepared according to recombinant molecular biology techniques with which a skilled artisan is quite familiar. One means to minimize immunogenicity of a CD47-Fc fusion polypeptide is to fuse the CD47 moiety to an Fc polypeptide using the nucleotide sequence and the encoded amino acid sequence derived from the animal species for whose use the Fc fusion polypeptide is intended. In one embodiment, the Fc polypeptide is of human origin and may be from any one of the human immunoglobulin classes, including, for example, human IgG1 and IgG2.

An Fc polypeptide that is a mutein Fc polypeptide may also be referred to as a Fc polypeptide variant. One such Fc polypeptide variant has one or more cysteine residues (such as one or more cysteine residues in the hinge region that forms an interchain disulfide bond) substituted with another amino acid, such as serine, to reduce the number of interchain disulfide bonds that can form between the two heavy chain constant region polypeptides. For example, the cysteine residue most proximal to the amino terminal end of the hinge region, which forms a disulfide bond with a light chain constant region to form a whole immunoglobulin molecule, may be substituted, for example, with a serine residue. Alternatively, any one or more cysteine residues, including the cysteine residue most proximal to the amino terminus of the hinge region, may be deleted from the hinge region of the Fc polypeptide.

In one embodiment, a mutein Fc polypeptide comprises a mutation of at least one cysteine residue in the hinge region of an Fc polypeptide. For certain immunoglobulin Fc portions, the cysteine residue in the mutein Fc that is substituted or deleted is the cysteine residue that is most proximal to the amino terminus of the hinge region of an Fc polypeptide (e.g., for example, the cysteine residue most proximal to the amino terminus of the hinge region of the Fc portion of a wildtype human IgG1 immunoglobulin). By way of illustration, the hinge of a human IgG1 Fc polypeptide has three cysteine residues (see, e.g., positions 1, 7, and 10 of SEQ ID NO:6). In certain embodiments, the cysteine residue that is most proximal to the amino terminal end of the human IgG1 Fc polypeptide, which corresponds to a cysteine residue at position 1 of SEQ ID NO:6, is deleted. Alternatively, the cysteine residue at this position is substituted with another amino acid that is incapable of forming a disulfide bond, for example, a serine residue.

In another embodiment, a mutein Fc polypeptide that comprises a deletion or substitution of the cysteine residue most proximal to the amino terminus of the hinge region of an Fc polypeptide further comprises a deletion or substitution of the adjacent amino acid that is toward the carboxy terminus (i.e., the adjacent C-terminal amino acid). In a certain embodiment, the cysteine residue most proximal to the amino terminus of the hinge portion and the adjacent C-terminal residue are both deleted from the hinge region of a mutein Fc polypeptide. In a specific embodiment, the mutein Fc polypeptide comprises a deletion of a cysteine residue that corresponds to the cysteine at position 1 of SEQ ID NO:6 and a deletion of an aspartic acid that corresponds to the aspartic acid at position 2 of SEQ ID NO:6. Fc polypeptides that comprise deletion of these cysteine and aspartic acid residues in the hinge region may be efficiently expressed in a host cell, and in certain instances, may be more efficiently expressed in a cell than an Fc polypeptide that retains the wildtype cysteine and aspartate residues.

In other embodiments, the Fc polypeptide may comprise at least one (i.e., one or more) substitutions, deletions, or insertions that increase or enhance the capability of the Fc polypeptide to alter the immunoresponsiveness of an immune cell. In particular embodiments, a substitution, deletion, or insertion of an amino acid is introduced into an Fc polypeptide to enhance or increase the capability of a CD47 extracellular domain-Fc polypeptide fusion, protein to suppress the immunoresponsiveness of an immune cell, and thus, in certain embodiments, to enhance the capability of the fusion protein to treat an immune disease or disorder.

In a certain embodiment, the Fc polypeptide is mutated to decrease the capability of the Fc polypeptide moiety of the fusion protein to bind to an Fc receptor that is expressed by an immune cell. Without wishing to be bound by theory, to decrease or abrogate the capability of the Fc polypeptide to bind to an Fc receptor of an immune cell, may decrease, minimize, or abrogate a signal that activates immunoresponsiveness of the immune cell in a manner that is undesired.

In one certain embodiment, Fc polypeptide may be an aglycosylated Fc polypeptide. Any one or more of an N-glycosylation site or an O-glycosylation site present in the Fc polypeptide may be removed by introducing one or more substitutions or deletions of an amino acid residue that may be glycosylated. For example, the asparagine residue of an N-linked glycosylation site (i.e., Asn-X-Ser/Thr, wherein X is any amino acid except Pro or Asp) may be substituted with another amino acid. The asparagine residue at position 219 of SEQ ID NO:2 corresponds to the asparagine residue at position 297 of a human IgG1 Fc polypeptide that is described in the art as being glycosylated (see Asn at position 78 of SEQ ID NO:6, an exemplary wildtype human IgG1 Fc polypeptide sequence). Thus an exemplary CD47 extracellular-Fc fusion polypeptide comprises the amino acid sequence set forth in SEQ ID NO:28. In another embodiment, the lysine residue at the N-terminus of the hinge region (see lysine at position 3 of SEQ ID NO6) may be deleted (see, e.g., SEQ ID NO:29, which has a deletion of the lysine residue located at position 144 in SEQ ID NO:2). In certain other embodiments, the Fc polypeptide comprises a substitution of the asparagine residue that corresponds to position 219 of SEQ ID NO:2 and deletion (or substitution) of the lysine residue at position 144 of SEQ ID NO:2.

The capability of a CD47 extracellular domain-Fc polypeptide fusion protein to alter, preferably suppress, the immunoresponsiveness of an immune cell may be increased or enhanced by incorporation of a linker polypeptide between the CD47 moiety and the Fc polypeptide moiety. Without wishing to be bound by any particular theory, a linker polypeptide may increase the flexibility, or remove constraint, of the CD47 extracellular domain moiety (which includes a CD47 extracellular domain dimer) to adopt an effective or more effective conformation to interact with an immune cell and affect the cell's immunoresponsiveness, or to interact with a molecule that, in turn, interacts with an immune cell to affect the immunoresponsiveness of the immune cell.

A CD47 extracellular domain (or variant or fragment thereof) fused in frame with an Fc polypeptide or Fc polypeptide variant (e.g., a mutein Fc polypeptide) may comprise a polypeptide linker or spacer sequence between the CD47 extracellular domain polypeptide and Fc polypeptide. The linker (or spacer) may be a single amino acid (such as for example a glycine residue) or may be two, three, four, five, six, seven, eight, nine, or ten amino acids, or may be any number of amino acids between 5 and 100 amino acids, between 5 and 50, 5 and 30, or 5 and 20 amino acids. A polypeptide linker may also include a short peptide linker that may comprise at least two amino acids that are encoded by a nucleotide sequence that is a restriction enzyme recognition site. Examples of such restriction enzyme recognition sites include, for example, BamHI, ClaI, EcoRI, HindIII, KpnI, NcoI, NheI, PmlI, PstI, SalI, and XhoI.

Thus, polypeptide linkers may separate the CD47 extracellular domain moiety from the Fc polypeptide moiety by a distance sufficient to aid or ensure that each polypeptide moiety properly folds into the secondary and tertiary structures necessary for the desired biological activity. By way of example, the linker should permit the extracellular domain of CD47 to assume the proper spatial orientation to form a binding site for a CD47 ligand. Suitable polypeptide linkers may adopt a flexible extended conformation that does not exhibit a propensity for developing an ordered secondary structure that could interact or interfere with the functional protein domains, and that also would have a minimal hydrophobic or charged character, which could promote an undesirable interaction with the functional CD47 domain. Typical surface amino acids in flexible protein regions include glycine (Gly), asparagine (Asn) and serine (Ser). Virtually any permutation of amino acid sequences containing Gly, Asn, and Ser would be expected to satisfy the above criteria for a peptide linker sequence. Other near-neutral amino acids, such as threonine (Thr) and alanine (Ala), may also be used in the linker sequence. Suitable polypeptide linkers (or spacer peptides) may comprise from 5 to 100 amino acids and in certain embodiments, comprise from 5 to 20 amino acids in length. Examples of such linkers include, but are not limited to (Gly₄ Ser (SEQ ID NO:30))ₙ, wherein n=1-12 or n=1-8, or n=1-4; Gly₄ SerGly₅ Ser (SEQ ID NO:31), and (Gly₄ SerGly₅ Ser) (SEQ ID NO:31)ₘ wherein m=2-4. See also SEQ ID NOS:32 and 33 comprising a human CD47 extracellular domain fused to an Fc polypeptide (Gly₄ Ser) linker wherein n=1 and 2, respectively.

As described herein, in certain embodiments, a mutein Fc polypeptide that is fused with a CD47 extracellular domain, or a variant thereof, comprises a substitution or a deletion of the cysteine residue that is most proximal to the amino terminus of the hinge region of an Fc polypeptide and a deletion of the adjacent aspartic acid residue (toward the C-terminal end of the Fc polypeptide). In other embodiments, the fusion polypeptide may further comprise at least one, two, or three or more amino acid substitutions in the CH2 domain of the Fc polypeptide that reduce the capability of the mutein Fc polypeptide to bind to an IgFc receptor. In specific embodiments, the at least one, two, or three amino acids substitutions in the CH2 domain are substitutions of an amino acid(s) located at a position that corresponds to EU numbered position 234, 235, and/or 237.

In another embodiment, a mutein Fc polypeptide is an Fc polypeptide variant that has at least one, two, three, four, five, six, seven, or more amino acids involved in an effector function substituted or deleted such that the Fc polypeptide has a reduced level of an effector function. The Fc portion of the immunoglobulin mediates certain effector functions of an immunoglobulin. Three general categories of effector functions associated with the Fc region include (1) activation of the classical complement cascade, (2) interaction with effector cells, and (3) compartmentalization of immunoglobulins. Such a mutein Fc polypeptide may also comprise the mutations in the hinge region described herein and/or may comprise one or more mutations that alter the glycosylation pattern of the Fc polypeptide.

Amino acids in the Fc region may be substituted to reduce or abrogate binding of the Fc polypeptide to a component of the complement cascade (see, e.g., Duncan et al., Nature 332:563-64 (1988), Morgan et al., Immunology 86:319-24 (1995)); or to reduce or abrogate the ability of the Fc polypeptide to bind to an IgG Fc receptor expressed by an immune cell (Wines et al., J. Immunol. 164:5313-18 (2000); Chappel et al., Proc. Natl. Acad. Sci. USA 88:9036 (1991); Canfield et al., J. Exp. Med. 173:1483 (1991); Duncan et al., supra); or to alter antibody-dependent cellular cytotoxicity. Such an Fc polypeptide variant that differs from the wildtype Fc polypeptide is also called herein a mutein Fc polypeptide.

In one embodiment, a CD47 extracellular domain or variant thereof is fused in frame with an Fc polypeptide that comprises at least one substitution of a residue that in the wildtype Fc region polypeptide contributes to binding of an Fc polypeptide or immunoglobulin to one or more IgG Fc receptors expressed on certain immune cells. Such a mutein Fc polypeptide comprises at least one substitution of an amino acid residue in the CH2 domain of the mutein Fc polypeptide, such that the capability of the fusion polypeptide to bind to an IgG Fc receptor, such as an IgG Fc receptor present on the surface of an immune cell, is reduced.

By way of background, on human leukocytes three distinct types of Fc IgG-receptors are expressed that are distinguishable by structural and functional properties, as well as by antigenic structures, which differences are detected by CD specific monoclonal antibodies. The IgG Fc receptors are designated FcγRI (CD64), FcγRII (CD32), and FcγRIII (CD16) and are differentially expressed on overlapping subsets of leukocytes.

FcγRI (CD64), a high-affinity receptor expressed on monocytes, macrophages, neutrophils, myeloid precursors, and dendritic cells, comprises isoforms 1a and 1b. FcγRII (CD32), comprised of isoforms IIa, IIb1, IIb2, IIb3, and IIc, is a low-affinity receptor that is the most widely distributed human FcγR type; it is expressed on most types of blood leukocytes, as well as on Langerhans cells, dendritic cells, and platelets. FcγRIII (CD16) has two isoforms, both of which are capable of binding to human IgG1 and IgG3. The FcγRIIIa isoform has an intermediate affinity for IgG and is expressed on macrophages, monocytes, natural killer (NK) cells, and subsets of T cells. FcγRIIIb is a low-affinity receptor for IgG and is selectively expressed on neutrophils.

Residues in the amino terminal portion of the CH2 domain that contribute to IgG Fc receptor binding include residues at positions between Leu234-Ser239 (Leu-Leu-Gly-Gly-Pro-Ser (SEQ ID NO:21) (EU numbering system, Kabat et al., supra) (see, e.g., Morgan et al., Immunology 86:319-24 (1995), and references cited therein). The position of these amino acids in an IgG immunoglobulin is designated using the EU numbering system, which is a widely used nomenclature in the antibody art when referring to residues of the Fc portion of an IgG immunoglobulin that bind to an IgG Fc receptor. The residues Leu234-Ser239 correspond to positions 15-20 of the amino acid sequence of a human IgG1 Fc polypeptide (SEQ ID NO:6). Substitution of the amino acid at one or more of these six positions (i.e., one, two, three, four, five, or all six) in the CH2 domain results in a reduction of the capability of the Fc polypeptide to bind to one or more of the IgG Fc receptors (or isoforms thereof) (see, e.g., Burton et al., Adv. Immunol. 51:1 (1992); Hulett et al., Adv. Immunol. 57:1 (1994); Jefferis et al., Immunol. Rev. 163:59 (1998); Lund et al., J. Immunol. 147:2657 (1991); Sarmay et al., Mol. Immunol. 29:633 (1992); Lund et al., Mol. Immunol. 29:53 (1992); Morgan et al., supra). In addition to substitution of one or more amino acids at EU positions 234-239, one, two, or three or more amino acids adjacent to this region (either to the carboxy terminal side of position 239 or to the amino terminal side of position 234) may also be substituted.

By way of example, substitution of the leucine residue at position 235 (which corresponds to position 16 of SEQ ID NO:6) with a glutamic acid residue or an alanine residue abolishes or reduces, respectively, the affinity of an immunoglobulin (such as human IgG3) for FcγRI (Lund et al., 1991, supra; Canfield et al., supra; Morgan et al., supra). As another example, replacement of the leucine residues at positions 234 and 235 (which correspond to positions 15 and 16 of SEQ ID NO:6), for example, with alanine residues, abrogates binding of an immunoglobulin to FcγRIIa (see, e.g., Wines et al., supra). Alternatively, leucine at position 234 (which corresponds to position 15 of SEQ ID NO:6), leucine at position 235 (which corresponds to position 16 of SEQ ID NO:6), and glycine at position 237 (which corresponds to position 18 of SEQ ID NO:6), each may be substituted with a different amino acid, such as leucine at position 234 may be substituted with an alanine residue (L234A), leucine at 235 may be substituted with an alanine residue (L235A) or with a glutamic acid residue (L235E), and the glycine residue at position 237 may be substituted with another amino acid, for example an alanine residue (G237A).

In one embodiment, a mutein Fc polypeptide that is fused in frame to a CD47 extracellular domain (or variant thereof) comprises one, two, three, four, five, or six mutations at positions 15-20 of SEQ ID NO:6 that correspond to positions 234-239 of a human IgG1 CH2 domain (EU numbering system) as described herein. An exemplary mutein Fc polypeptide has the amino acid sequence set forth in SEQ ID NO:7 in which substitutions corresponding to (L234A), (L235E), and (G237A) may be found at positions 13, 14, and 16 of SEQ ID NO:7.

In a specific embodiment, a mutein Fc polypeptide comprises the amino acid sequence set forth in SEQ ID NO:7, which differs from the wildtype Fc polypeptide (SEQ ID NO:6) wherein the cysteine residue at position 1 of SEQ ID NO:6 is deleted and the aspartic acid at position 2 of SEQ ID NO:6 is deleted and the leucine reside at position 15 of SEQ ID NO:6 is substituted with an alanine residue, the leucine residue at position 16 is substituted with a glutamic acid residue, and the glycine at position 18 is substituted with an alanine residue. Thus, an exemplary mutein Fc polypeptide comprises an amino acid sequence at its amino terminal portion of KTHTCPPCPAPEAEGAPS (SEQ ID NO:22) (see SEQ ID NO:7, an exemplary Fc mutein sequence). Thus, an exemplary Fc mutein polypeptide comprises deletion of a cysteine residue most proximal to the amino terminus of the hinge, a deletion of the aspartate residue adjacent to this cysteine, and substitutions of amino acids at positions that correspond to EU234, EU235, and EU237.

Other Fc variants encompass similar amino acid sequences of known Fc polypeptide sequences that have only minor changes, for example by way of illustration and not limitation, covalent chemical modifications, insertions, deletions and/or substitutions, which may further include conservative substitutions. Amino acid sequences that are similar to one another may share substantial regions of sequence homology. Similarly, nucleotide sequences that encode the Fc variants may encompass substantially similar nucleotide sequences and have only minor changes, for example by way of illustration and not limitation, covalent chemical modifications, insertions, deletions, and/or substitutions, which may further include silent mutations owing to degeneracy of the genetic code. Nucleotide sequences that are similar to one another may share substantial regions of sequence homology.

The nucleotide sequences that encode Fc polypeptides from various classes and isotypes of immunoglobulins from various species are known and available in GenBank databases and in Kabat (Kabat et al., in Sequences of Proteins of Immunological Interest, 4th ed., (U.S. Dept. of Health and Human Services, U.S. Government Printing Office, 1991), see also updates to the online Kabat database available by license). Any of these polynucleotide sequences (or any degenerate polynucleotide sequence that encodes the Fc polypeptide of interest) may be used for preparing a recombinant construct according to molecular biology methods routinely practiced by persons skilled in the art. To minimize the immunogenicity of the Fc polypeptide in the host or subject to which a fusion polypeptide comprising a CD47 extracellular domain (or variant thereof) may be administered, the sequence of the Fc polypeptide is typically chosen from immunoglobulins of the same species, that is, for example, a human Fc polypeptide sequence is fused to a human CD47 extracellular domain, or variant thereof, that will be administered to a human subject or host.

In other embodiments, the fusion polypeptide comprises a viral CD47 extracellular domain, or variant thereof, as described herein, fused to any one of the mutein Fc polypeptides described above.

### Production of a Fusion Polypeptide Comprising a CD47 Extracellular Domain Fused to a Mutein Fc Polypeptide

Any one of the fusion polypeptides described herein that comprise a CD47 extracellular domain, or variant thereof, fused in frame to an Fc polypeptide, or variant thereof, or that comprise a viral CD47 extracellular domain, or variant thereof, fused in frame to a Fc polypeptide, or variant thereof, may be produced, made, or manufactured according to methods described herein and routinely practiced in the art. In certain embodiments, the fusion polypeptides are recombinant fusion polypeptides. A recombinant expression construct may be prepared for the expression of a fusion polypeptide according to standard techniques and methods practiced by a skilled person in the molecular biology art. In order to obtain efficient transcription and translation, the polynucleotide sequence in each construct includes appropriate regulatory sequences, particularly a promoter and leader sequence operatively linked to the nucleotide sequence encoding a CD47 extracellular domain, or variant thereof, or may be operatively linked to a nucleotide sequence encoding a signal peptide sequence located at the amino terminal end of the CD47 extracellular domain. The polynucleotide encoding the CD47 extracellular domain further may further comprise a nucleotide sequence that encodes a Fc polypeptide, including a mutein Fc polypeptide, that is expressed in frame with the CD47 extracellular domain, or variant thereof. In addition, as described herein, the polynucleotide may also encode, in frame, a polypeptide linker (or spacer peptide or polypeptide) between the CD47 moiety and the Fc polypeptide moiety.

Numerous vectors are available from commercial vendors for cloning and preparing recombinant expression constructs. Methods and techniques for producing polypeptides recombinantly are generally well known and routinely used. For example, molecular biology procedures are described by Sambrook et al. (Molecular Cloning, A Laboratory Manual, 2nd ed., Cold Spring Harbor Laboratory, New York, 1989; see also Sambrook et al., 3rd ed., Cold Spring Harbor Laboratory, New York, (2001)). As described herein, in certain embodiments, the fusion polypeptide further comprises a linker or spacer polypeptide sequence (a polypeptide sequence as described herein is intended to include a peptide sequence) between the CD47 extracellular domain, or variant thereof, and the Fc polypeptide. Persons skilled in the art can readily prepare polynucleotide sequences that encode a linker (or spacer), which linker may be a single amino acid (such as for example a glycine residue) or may be two, three, four, five, six, seven, eight, nine, or ten amino acids, or may be any number of amino acids between 5 and 100 amino acids, and in certain embodiments between 5 and 20 amino acids, which are described in greater detail herein. A polypeptide linker may also include a short peptide such as a peptide linker that is at least two amino acids that are encoded by a nucleotide sequence that is a restriction enzyme recognition site. Examples of such restriction enzyme recognition sites include, for example, BamHI, ClaI, EcoRI, HindIII, KpnI, NcoI, NheI, PmlI, PstI, SalI, and XhoI.

A recombinant expression construct may be prepared for the expression of a fusion polypeptide according to standard techniques and methods practiced by a skilled person in the molecular biology art. In order to obtain efficient transcription and translation, the polynucleotide sequence in each construct should include appropriate regulatory sequences, particularly a promoter and leader sequence operatively linked to the nucleotide sequence encoding the CD47 extracellular domain, or variant thereof, or may be operatively linked to a nucleotide sequence encoding the signal peptide sequence located at the amino terminal end of the CD47 extracellular domain. Particular methods for producing polypeptides recombinantly are generally well known and routinely used. For example, molecular biology procedures are described by Sambrook et al. (Molecular Cloning, A Laboratory Manual, 2nd ed., Cold Spring Harbor Laboratory, New York, 1989; see also Sambrook et al., 3rd ed., Cold Spring Harbor Laboratory, New York, (2001)).

A recombinant construct prepared according to methods routinely practiced in the art may be introduced into a host cell via any one of several transformation, transfection, or transduction methods. Suitable host cells include prokaryotes, insect cells, yeast, and higher eukaryotic cells (including mammalian cells). In certain other embodiments, the fusion polypeptide may be expressed in a eukaryotic host cell, including yeast (e.g., Saccharomyces cerevisiae, Schizosaccharomyces pombe, and Pichia pastoris); an animal cell (including mammalian cells) or plant cells. Examples of suitable animal cells include, for example, COS, CHO, or HEK293 cells. Examples of plant cells include tobacco, corn, soybean, and rice cells. By using methods known to those having ordinary skill in the art and based on the present disclosure, a nucleic acid vector may be designed for expressing foreign sequences in a particular host system, and then polynucleotide sequences encoding the fusion polypeptide may be inserted. The regulatory elements will vary according to the particular host. See, for example, products and methods provided by commercial vendors, for example, Invitrogen (Carlsbad, Calif.); Stratagene (San Diego, Calif.); and BioCarta Inc. (San Diego, Calif.).

Methods for manufacturing the CD47 Fc fusion polypeptides are also provided herein. Methods as described above may be adapted for larger scale manufacturing of the CD47 Fc polypeptide fusion proteins. Manufacturing the polypeptides may comprise growing host cells that express such polypeptides in bioreactors, which reactors may also include matrices to which the host cells may attach, which, without wishing to be bound by theory permits cell cultures at high density.

### Antibodies That Specifically Bind to CD47 and Antibodies that Specifically Bind to a CD47 Ligand

Also provided herein are antibodies, and antigen-binding fragments thereof, that specifically bind to the extracellular domain of CD47. The anti-CD47 antibodies described herein competitively inhibit binding of a CD47 ligand to CD47 and competitively inhibit binding of a CD47 ligand to viral CD47 (i.e., a viral CD-47 like polypeptide). A CD47 ligand includes, for example, SIRP-α, SIRP-beta-2, thrombospondin-1, αᵥβ₃ integrin, and α₂β₁ integrin. Without wishing to be bound by theory, an antibody, or antigen-binding fragment thereof, that competitively inhibits binding of viral CD47 to a CD47 ligand may manifest a similar immunosuppressive effect that occurs when viral CD47 is expressed by a cell that is infected with the poxvirus containing the genome that encodes the viral CD47. Thus, in other embodiments, antibodies, and antigen-binding fragments thereof, are provided herein that bind specifically to a CD47 ligand and that inhibit binding of a viral CD47 to bind to the CD47 ligand. An antibody that is specific for a CD47 ligand is understood to mean that the antibody binds specifically to one CD47 ligand, that is, an antibody is provided that specifically binds to the CD47 ligand SIRP-α. A different antibody, and antigen-binding fragment thereof, specifically binds to a second CD47 ligand such as thrombospondin-1. That is when an antibody, or antigen-binding fragment thereof, is referred to herein as an antibody that binds to a CD47 ligand, this means that the antibody has a single specificity and does not bind to multiple CD47 ligands.

Such an antibody, or antigen-binding fragment thereof, may modulate or alter the immune response of a host, and may particularly inhibit, suppress, or decrease the extent of, an immune response exhibited in an immunological disease or disorder, for example, an inflammatory or autoimmune disease or disorder. In certain embodiments, an anti-CD47 antibody, or antigen-binding fragment thereof, or an anti-CD47 ligand antibody, or antigen-binding fragment thereof, alters the ability of an accessory cell to release cytokines and/or to mature. An antibody that specifically binds to CD47 or an antibody that specifically binds to a CD47 ligand may have the capability to alter (enhance or suppress in a statistically significant or biologically significant manner) the immunoresponsiveness of an immune cell. Such an antibody or antigen binding fragment thereof may alter or affect the immunoresponsiveness of an immune cell by effecting a biological function or action, including any one or more (or at least one of) the following: inhibit maturation of dendritic cells; impair development of naïve T cells into Th1 effector cells; suppress cytokine release by dendritic cells; alter cell migration; inhibit production of at least one cytokine, for example, at least one of TNF-α, IL-12, IL-23, IFN-γ, GM-CSF, and IL-6; inhibit maturation of a dendritic cell; impair development of a naïve T cell into a Th1 effector cell; suppress cytokine secretion by a dendritic cell; inhibit production of a chemokine, for example MIP-1α; and suppress a proinflammatory response. An anti-CD47 ligand antibody that specifically binds to its cognate CD47 ligand may also inhibit an activity of function attributable to that CD47 ligand.

A viral CD47 may be any one of the poxvirus CD47-like polypeptides described herein or known in the art. Viral genomic sequences that encode viral CD47-like polypeptides have been identified in several poxviruses, including myxoma and orthopoxviruses as well as chordopoxvirus, a capripoxvirus, a leporipoxvirus, a suipoxvirus, a yatapoxvirus, and a deerpox virus. Exemplary amino acid sequences of poxvirus CD47-like polypeptides include the A44L polypeptide encoded by the genome of Variola minor virus (GenBank Accession No. CAB54747.1 (SEQ ID NO:3)); Vaccinia virus-Western Reserve (GenBank Accession No. AA089441.1); Vaccinia virus (modified virus Ankara) (GenBank Accession No. AAT10547.1); and Myxoma M128L polypeptide (GenBank Accession Nos. NP_{—}051842.1 and AAF15016.1).

The antibodies and antigen-binding fragment described herein that specifically bind to CD47.or that specifically bind to a CD47 ligand may be useful for altering immunoresponsiveness of an immune cell and thereby may be useful for treating or preventing an immunological disease or disorder, cardiovascular disease or disorder, metabolic disease or disorder, or a proliferative disease or disorder. An immunological disease or disorder may be an autoimmune disease or an inflammatory disease. In certain embodiments, the immunological disease or disorder is multiple sclerosis, rheumatoid arthritis, a spondyloarthropathy, systemic lupus erythematosus, an antibody-mediated inflammatory or autoimmune disease or disorder, graft versus host disease, sepsis, diabetes, psoriasis, atherosclerosis, Sjogren's syndrome, progressive systemic sclerosis, scleroderma, acute coronary syndrome, ischemic reperfusion, Crohn's Disease, endometriosis, glomerulonephritis, myasthenia gravis, idiopathic pulmonary fibrosis, asthma, acute respiratory distress syndrome (ARDS), vasculitis, or inflammatory autoimmune myositis. A spondyloarthropathy includes, for example, ankylosing spondylitis, reactive arthritis, enteropathic arthritis associated with inflammatory bowel disease, psoriatic arthritis, isolated acute anterior uveitis, undifferentiated spondyloarthropathy, Behcet's syndrome, and juvenile idiopathic arthritis. The fusion polypeptides described herein may also be useful for treating a cardiovascular disease or disorder, such as atherosclerosis, endocarditis, hypertension, or peripheral ischemic disease.

As described herein, the anti-CD47 antibodies or anti-CD47 ligand antibodies may be useful for treating or preventing, inhibiting, slowing the progression of, or reducing the symptoms associated with, an immunological disease or disorder, a cardiovascular disease or disorder, a metabolic disease or disorder, or a proliferative disease or disorder. An immunological disorder includes an inflammatory disease or disorder and an autoimmune disease or disorder. While inflammation or an inflammatory response is a host's normal and protective response to an injury, inflammation can cause undesired damage. For example, atherosclerosis is, at least in part, a pathological response to arterial injury and the consequent inflammatory cascade. A cardiovascular disease or disorder that may be treated, which may include a disease and disorder that is also considered an immunological disease/disorder, includes for example, atherosclerosis, endocarditis, hypertension, or peripheral ischemic disease. A metabolic disease or disorder includes diabetes, obesity, and diseases and disorders associated with abnormal or altered mitochondrial function.

Any one or more of the fusion polypeptides comprising a CD47 extracellular domain or variant thereof as described herein may also be used in methods for screening samples containing antibodies, for example, samples of purified antibodies, antisera, or cell culture supernatants, or any other biological sample that may contain one or more antibodies specific for one or more of the fusion polypeptides. One or more of the fusion polypeptides comprising a CD47 extracellular domain or variant thereof may also be used in methods for identifying and selecting from a biological sample one or more B cells that are producing an antibody that specifically binds to the extracellular domain of CD47 (e.g., plaque forming assays and the like). The B cells may then be used as source of the specific antibody-encoding polynucleotide that can be cloned and/or modified by recombinant molecular biology techniques known in the art and described herein.

As used herein, an antibody is said to be “immunospecific,” “specific for” or to “specifically bind” a CD47 extracellular domain (or variant thereof) if it reacts at a detectable level with the CD47 extracellular domain (or variant thereof), preferably with an affinity constant, Kₐ, of greater than or equal to about 10⁴ M⁻¹, or greater than or equal to about 10⁵ M⁻¹, greater than or equal to about 10⁶ M⁻¹, greater than or equal to about 10⁷ M⁻¹, or greater than or equal to 10⁸ M⁻¹. Affinity of an antibody for its cognate antigen is also commonly expressed as a dissociation constant K_{D}, and an anti-CD47 extracellular domain (or variant thereof) antibody, for example, specifically binds to CD47 if it binds with a K_{D} of less than or equal to 10⁻⁴ M, less than or equal to about 10⁻⁵ M, less than or equal to about 10⁻⁶ M, less than or equal to 10⁻⁷ M, or less than or equal to 10⁻⁸ M.

Affinities of binding partners or antibodies can be readily determined using conventional techniques, for example, those described by Scatchard et al. (Ann. N.Y. Acad. Sci. USA 51:660 (1949)) and by surface plasmon resonance (SPR; BIAcore™, Biosensor, Piscataway, N.J.). For surface plasmon resonance, target molecules are immobilized on a solid phase and exposed to ligands in a mobile phase running along a flow cell. If ligand binding to the immobilized target occurs, the local refractive index changes, leading to a change in SPR angle, which can be monitored in real time by detecting changes in the intensity of the reflected light. The rates of change of the surface plasmon resonance signal can be analyzed to yield apparent rate constants for the association and dissociation phases of the binding reaction. The ratio of these values gives the apparent equilibrium constant (affinity) (see, e.g., Wolff et al., Cancer Res. 53:2560-2565 (1993)).

Binding properties of an antibody to CD47 (particularly, the CD47 extracellular domain) or of an antibody that binds to a CD47 ligand may generally be determined and assessed using immunodetection methods including, for example, an enzyme-linked immunosorbent assay (ELISA), immunoprecipitation, immunoblotting, countercurrent immunoelectrophoresis, radioimmunoassays, dot blot assays, inhibition or competition assays, and the like, which may be readily performed by those having ordinary skill in the art (see, e.g., U.S. Pat. Nos. 4,376,110 and 4,486,530; Harlow et al., Antibodies: A Laboratory Manual, Cold Spring Harbor Laboratory (1988)). Immunoassay methods may include controls and procedures to determine whether antibodies bind specifically to CD47 and do not recognize or cross-react with unrelated polypeptides. Similarly, immunoassay methods may include controls and procedures to determine that an antibody that bind specifically to a CD47 ligand does not recognize or cross-react with unrelated polypeptides. In addition, an immunoassay performed for detection of anti-CD47 antibodies that are produced in response to immunization of a host with CD47, or a CD47 extracellular domain, conjugated to a particular carrier polypeptide may incorporate the use of the extracellular domain that is conjugated to a different carrier polypeptide than that used for immunization to reduce or eliminate detection of antibodies that bind specifically to the immunizing carrier polypeptide. Alternatively, CD47 or a CD47 extracellular domain that is not conjugated to a carrier molecule may be used in an immunoassay for detecting immunospecific antibodies.

Antibodies may generally be prepared by any of a variety of techniques known to persons having ordinary skill in the art. See, e.g., Harlow et al., Antibodies. A Laboratory Manual, Cold Spring Harbor Laboratory (1988); Peterson, ILAR J 46:314-19 (2005)). Isolated CD47, the extracellular domain of CD47, peptides or fragments thereof, or a cell expressing CD47 may be used as an immunogen for immunizing an animal for production of either polyclonal antibodies or monoclonal antibodies.

An immunogen may comprise a portion of the extracellular region, which may be used to generate and/or identify antibodies or antigen-binding fragments thereof that are capable of altering (increasing or decreasing in a statistically significant or biological significant manner, preferably decreasing) the immunoresponsiveness of an immune cell. Exemplary peptide immunogens may comprise 6, 7, 8, 9, 10, 11, 12, 20-25, 21-50, 26-30, 31-40, 41-50, 51-60, 61-70, or 71-75 consecutive amino acids of a CD47 extracellular domain. Similarly, peptide immunogens may be prepared from the extracellular domains of a CD47 ligand.

Peptides and fragments of the extracellular domain of CD47 that are useful as immunogens include portions of the extracellular domain that have a binding site to which a CD47 ligand binds. One method for determining the amino acid sequence of a CD47 ligand binding site, or a portion of the ligand binding site, includes peptide mapping techniques. For example, peptides may be randomly generated by proteolytic digestion of the extracellular domain of CD47 using any one or more of various proteases, the peptides separated and/or isolated (e.g., by gel electrophoresis, column chromatography), followed by determination of which peptide(s) a CD47 ligand binds to, and then sequencing the peptides. The CD47 extracellular domain peptides may also be generated using recombinant methods described herein and practiced in the art. Peptides randomly generated by recombinant methods may also be used to prepare peptide combinatorial libraries or phage libraries as described herein and in the art. Alternatively, the amino acid sequences of portions of the extracellular domain of CD47 that interact with a CD47 ligand may be determined by computer modeling of CD47, or of a portion thereof, for example, the extracellular portion, and/or by x-ray crystallography (which may include preparation and analysis of crystals of the CD47 extracellular domain only or of the extracellular domain CD47-CD47 ligand complex). Conversely, peptides and fragments of the extracellular domain of a CD47 ligand that are useful as immunogens include portions of the extracellular domain that have a binding site to which CD47 and/or to which vCD47 binds and may be identified as described above for the CD47 peptides.

Immunogenic peptides of a CD47 extracellular domain (or immunogenic peptides of a CD47 ligand extracellular domain) may also be determined by computer analysis of the amino acid sequence of the domain to determine a hydrophilicity plot. Portions of the extracellular domain that are accessible to an antibody are most likely portions of the protein that are in contact with the aqueous environment and are hydrophilic. Regions of hydrophilicity can be determined using computer programs available to persons skilled in the art and which assign a “hydrophilic index” to each amino acid in a protein and then plot a profile.

Preparation of an Immunogen, Particularly Polypeptide Fragments or Peptides, for injection into animals may include covalent coupling of the CD47 extracellular domain (or variant thereof) or peptide, (or the CD47 ligand extracellular domain or peptide thereof) to another immunogenic protein, for example, a carrier protein such as keyhole limpet hemocyanin (KLH) or bovine serum albumin (BSA) or the like. A polypeptide or peptide immunogen may include one or more additional amino acids at either the N-terminal or C-terminal end that facilitate the conjugation procedure (e.g., the addition of a cysteine to facilitate conjugation of a peptide to KLH). Other amino acid residues within a polypeptide or peptide may be substituted to prevent conjugation at that particular amino acid position to a carrier polypeptide (e.g., substituting a serine residue for cysteine at internal positions of a polypeptide/peptide) or may be substituted to facilitate solubility or to increase immunogenicity.

An antibody that specifically binds to CD47 and an antibody that specifically binds to a CD47 ligand may belong to any immunoglobulin class, for example IgG, IgE, IgM, IgD, or IgA. It may be obtained from or derived from an animal, for example, fowl (e.g., chicken) and mammals, which include but are not limited to a mouse, rat, hamster, rabbit, or other rodent, a cow, horse, sheep, goat, camel, human, or other primate. The antibody may be an internalising antibody. In one such technique, an animal is immunized, for example, with an extracellular domain or fragment thereof as described herein as an antigen to generate polyclonal antisera. Suitable animals include, for example, rabbits, sheep, goats, pigs, cattle, and may also include smaller mammalian species, such as mice, rats, and hamsters, or other species.

Polyclonal antibodies that bind specifically to an antigen, such as CD47 or a CD47 ligand, can be prepared using methods described herein and practiced by persons skilled in the art (see, for example, Green et al., “Production of Polyclonal Antisera,” in Immunochemical Protocols (Manson, ed.), pages 1-5 (Humana Press 1992); Harlow et al., Antibodies. A Laboratory Manual, Cold Spring Harbor Laboratory (1988); Williams et al., “Expression of foreign proteins in E. coli using plasmid vectors and purification of specific polyclonal antibodies,” in DNA Cloning 2: Expression Systems, 2nd Edition, Glover et al. (eds.), page 15 (Oxford University Press 1995)). Although polyclonal antibodies are typically raised in animals such as rats, mice, rabbits, goats, cattle, or sheep, an antibody may also be obtained from a subhuman primate. General techniques for raising diagnostically and therapeutically useful antibodies in baboons may be found, for example, in International Patent Application Publication No. WO 91/11465 (1991) and in Losman et al., Int. J Cancer 46:310, 1990.

In addition, the antigen (CD47, the CD47 extracellular domain polypeptide, fragment or peptide thereof, or a cell expressing CD47; a CD47 ligand, extracellular domain of the ligand or a cell expressing the ligand) used as an immunogen may be emulsified in an adjuvant. See, e.g., Harlow et al., Antibodies: A Laboratory Manual, Cold Spring Harbor Laboratory (1988). Adjuvants typically used for immunization of non-human animals include but are not limited to Freund's complete adjuvant, Freund's incomplete adjuvant, montanide ISA, Ribi Adjuvant System (RAS) (Corixa Corporation, Seattle, Wash.), and nitrocellulose-adsorbed antigen. The immunogen may be injected into the animal via any number of different routes, including intraperitoneally, intravenously, intramuscularly, intradermally, intraocularly, or subcutaneously. In general, after the first injection, animals receive one or more booster immunizations according to a preferred schedule that may vary according to, inter alia, the antigen, the adjuvant (if any) and/or the particular animal species. The immune response may be monitored by periodically bleeding the animal, separating the sera from the collected blood, and analyzing the sera in an immunoassay, such as an ELISA or Ouchterlony diffusion assay, or the like, to determine the specific antibody titer. Once an adequate antibody titer is established, the animals may be bled periodically to accumulate the polyclonal antisera. Polyclonal antibodies that bind specifically to the antigen may then be purified from such antisera, for example, by affinity chromatography using protein A or protein G immobilized on a suitable solid support (see, e.g., Coligan, supra, p. 2.7.1-2.7.12; 2.9.1-2.9.3; Baines et al., Purification of Immunoglobulin G (IgG), in Methods in Molecular Biology, 10:9-104 (The Humana Press, Inc. (1992)). Alternatively, affinity chromatography may be performed wherein the antigen, or a fragment thereof, or an antibody specific for an Ig constant region of the particular immunized animal species is immobilized on a suitable solid support.

Monoclonal antibodies that specifically bind to CD47 and particularly, to the CD47 extracellular domain, or monoclonal antibodies specific for a CD47 ligand, and hybridomas, which are examples of immortal eukaryotic cell lines, that produce monoclonal antibodies having the desired binding specificity, may also be prepared, for example, using the technique of Kohler and Milstein (Nature, 256:495-97 (1976), Eur. J. Immunol. 6:511-19 (1975)) and improvements thereto (see, e.g., Coligan et al. (eds.), Current Protocols in Immunology, 1:2.5.1-2.6.7 (John Wiley & Sons 1991); U.S. Pat. Nos. 4,902,614, 4,543,439, and 4,411,993; Monoclonal Antibodies, Hybridomas. A New Dimension in Biological Analyses, Plenum Press, Kennett et al. (eds.) (1980); and Antibodies: A Laboratory Manual, Harlow and Lane (eds.), Cold Spring Harbor Laboratory Press (1988); see also, e.g., Brand et al., Planta Med. 70:986-92 (2004); Pasqualini et al., Proc. Natl. Acad. Sci. USA 101:257-59 (2004)). An animal, for example, a rat, hamster, or more commonly, a mouse, is immunized with an immunogen prepared as described above. The presence of specific antibody production may be monitored after the initial injection (injections may be administered by any one of several routes as described herein for generation of polyclonal antibodies) and/or after a booster injection by obtaining a serum sample and detecting the presence of an antibody that binds to the antigen using any one of several immunodetection methods known in the art and described herein.

From animals producing specific antibodies, lymphoid cells (most commonly cells from the spleen or lymph node) are removed to obtain B-lymphocytes, which are lymphoid cells that are antibody-forming cells. The lymphoid cells, typically spleen cells, may be immortalized by fusion with a drug-sensitized myeloma (e.g., plasmacytoma) cell fusion partner, preferably one that is syngeneic with the immunized animal and that optionally has other desirable properties (e.g., inability to express endogenous Ig gene products, e.g., P3X63-Ag 8.653 (ATCC No. CRL 1580); NS0; SP20)). The lymphoid cells and the myeloma cells may be combined for a few minutes with a membrane fusion-promoting agent, such as polyethylene glycol or a nonionic detergent, and then plated at low density on a selective medium that supports the growth of hybridoma cells, but not unfused myeloma cells. A preferred selection media is HAT (hypoxanthine, aminopterin, thymidine). After a sufficient time, usually about one to two weeks, colonies of cells are observed. Antibodies produced by the cells may be tested for binding activity to the antigen. The hybridomas are cloned (e.g., by limited dilution cloning or by soft agar plaque isolation) and positive clones that produce an antibody specific to the antigen are selected and cultured. Hybridomas producing monoclonal antibodies with high affinity and specificity for the CD47 extracellular domain are preferred. Similarly, hybridomas producing monoclonal antibodies with high affinity and specificity for the CD47 ligand, particularly, the extracellular domain, are preferred.

Monoclonal antibodies may be isolated from the supernatants of hybridoma cultures. An alternative method for production of a murine monoclonal antibody is to inject the hybridoma cells into the peritoneal cavity of a syngeneic mouse, for example, a mouse that has been treated (e.g., pristane-primed) to promote formation of ascites fluid containing the monoclonal antibody. Contaminants may be removed from the subsequently harvested ascites fluid (usually within 1-3 weeks) by conventional techniques, such as chromatography (e.g., size-exclusion, ion-exchange), gel filtration, precipitation, extraction, or the like (see, e.g., Coligan, supra, p. 2.7.1-2.7.12; 2.9.1-2.9.3; Baines et al., Purification of Immunoglobulin G (IgG), in Methods in Molecular Biology, 10:9-104 (The Humana Press, Inc. (1992)). For example, antibodies may be purified by affinity chromatography using an appropriate ligand selected based on particular properties of the monoclonal antibody (e.g., heavy or light chain isotype, binding specificity, etc.). Examples of a suitable ligand, immobilized on a solid support, include Protein A, Protein G, an anti-constant region (light chain or heavy chain) antibody, an anti-idiotype antibody, the CD47 extracellular domain or fragment thereof.

An antibody that specifically binds to CD47 or an antibody that specifically binds to a CD47 ligand may be a human monoclonal antibody. Human monoclonal antibodies may be generated by any number of techniques with which those having ordinary skill in the art will be familiar. Such methods include, but are not limited to, Epstein Barr Virus (EBV) transformation of human peripheral blood cells (e.g., containing B lymphocytes), in vitro immunization of human B cells, fusion of spleen cells from immunized transgenic mice carrying inserted human immunoglobulin genes, isolation from human immunoglobulin V region phage libraries, or other procedures as known in the art and based on the disclosure herein.

For example, human monoclonal antibodies may be obtained from transgenic mice that have been engineered to produce specific human antibodies in response to antigenic challenge. Methods for obtaining human antibodies from transgenic mice are described, for example, by Green et al., Nature Genet. 7:13 (1994); Lonberg et al., Nature 368:856 (1994); Taylor et al., Int. Immun. 6:579 (1994); U.S. Pat. No. 5,877,397; Bruggemann et al., Curr. Opin. Biotechnol. 8:455-58 (1997); Jakobovits et al., Ann. N.Y. Acad. Sci. 764:525-35 (1995). In this technique, elements of the human heavy and light chain locus are artificially introduced by genetic engineering into strains of mice derived from embryonic stem cell lines that contain targeted disruptions of the endogenous murine heavy chain and light chain loci. (See also Bruggemann et al., Curr. Opin. Biotechnol. 8:455-58 (1997)). For example, human immunoglobulin transgenes may be mini-gene constructs, or transloci on yeast artificial chromosomes, which undergo B cell-specific DNA rearrangement and hypermutation in the mouse lymphoid tissue. Human monoclonal antibodies may be obtained by immunizing the transgenic mice, which may then produce human antibodies specific for the antigen. Lymphoid cells of the immunized transgenic mice can be used to produce human antibody-secreting hybridomas according to the methods described herein. Polyclonal sera containing human antibodies may also be obtained from the blood of the immunized animals.

Another method for generating human antigen-specific monoclonal antibodies includes immortalizing human peripheral blood cells by EBV transformation. See, e.g., U.S. Pat. No. 4,464,456. Such an immortalized B cell line (or lymphoblastoid cell line) producing a monoclonal antibody that specifically binds to the CD47 extracellular domain can be identified by immunodetection methods as provided herein, for example, an ELISA, and then isolated by standard cloning techniques. The stability of the lymphoblastoid cell line producing an antibody of intereset may be improved by fusing the transformed cell line with a murine myeloma to produce a mouse-human hybrid cell line according to methods known in the art (see, e.g., Glasky et al., Hybridoma 8:377-89 (1989)). Still another method to generate human monoclonal antibodies is in vitro immunization, which includes priming human splenic B cells with antigen, followed by fusion of primed B cells with a heterohybrid fusion partner. See, e.g., Boerner et al., J. Immunol. 147:86-95 (1991).

In certain embodiments, a B cell that is producing the desired antibody is selected, and the light chain and heavy chain variable regions are cloned from the B cell according to molecular biology techniques known in the art (WO 92/02551; U.S. Pat. No. 5,627,052; Babcook et al., Proc. Natl. Acad. Sci. USA 93:7843-48 (1996)) and described herein. B cells from an immunized animal are isolated from the spleen, lymph node, or peripheral blood sample by selecting a cell that is producing an antibody that specifically binds to the antigen, for example, the CD47 extracellular domain or a CD47 ligand. B cells may also be isolated from humans, for example, from a peripheral blood sample. Methods for detecting single B cells that are producing an antibody with the desired specificity are well known in the art, for example, by plaque formation, fluorescence-activated cell sorting, in vitro stimulation followed by detection of specific antibody, and the like. Methods for selection of specific antibody producing B cells include, for example, preparing a single cell suspension of B cells in soft agar that contains the antigen or a fragment thereof. Binding of the specific antibody produced by the B cell to the antigen results in the formation of a complex, which may be visible as an immunoprecipitate. After the B cells producing the specific antibody are selected, the specific antibody genes may be cloned by isolating and amplifying DNA or mRNA according to methods known in the art and described herein.

Chimeric antibodies, including humanized antibodies, may also be generated. A chimeric antibody has at least one constant region domain derived from a first mammalian species and at least one variable region domain derived from a second, distinct mammalian species. See, e.g., Morrison et al., Proc. Natl. Acad. Sci. USA, 81:6851-55 (1984). In one embodiment, a chimeric antibody may be constructed by cloning the polynucleotide sequence that encodes at least one variable region domain derived from a non-human monoclonal antibody, such as the variable region derived from a murine, rat, or hamster monoclonal antibody, into a vector containing a nucleic acid sequence that encodes at least one human constant region (see, e.g., Shin et al., Methods Enzymol. 178:459-76 (1989); Walls et al., Nucleic Acids Res. 21:2921-29 (1993)). By way of example, the polynucleotide sequence encoding the light chain variable region of a murine monoclonal antibody may be inserted into a vector containing a nucleic acid sequence encoding the human kappa light chain constant region sequence. In a separate vector, the polynucleotide sequence encoding the heavy chain variable region of the monoclonal antibody may be cloned in frame with sequences encoding the human IgG1 constant region. The particular human constant region selected may depend upon the effector functions desired for the particular antibody (e.g., complement fixing, binding to a particular Fc receptor, etc.). Another method known in the art for generating chimeric antibodies is homologous recombination (e.g., U.S. Pat. No. 5,482,856). Preferably, the vectors will be transfected into eukaryotic cells for stable expression of the chimeric antibody.

A non-human/human chimeric antibody may be further genetically engineered to create a “humanized” antibody. Such a humanized antibody may comprise a plurality of CDRs derived from an immunoglobulin of a non-human mammalian species, at least one human variable framework region, and at least one human immunoglobulin constant region. Humanization may in certain embodiments provide an antibody that has decreased binding affinity for the CD47 extracellular domain when compared, for example, with either a non-human monoclonal antibody from which a CD47-binding variable region is obtained, or a chimeric antibody having such a V region and at least one human C region, as described above. Humanization of an antibody that specifically binds to a CD47 ligand may be accomplished by similar methods described herein for preparing an anti-CD47 antibody. Useful strategies for designing humanized antibodies may therefore include, for example by way of illustration and not limitation, identification of human variable framework regions that are most homologous to the non-human framework regions of the chimeric antibody. Without wishing to be bound by theory, such a strategy may increase the likelihood that the humanized antibody will retain specific binding affinity for CD47, wherein the humanized antibody has substantially the same affinity as the non-humanized antibody, and in certain other embodiments the humanized antibody may exhibit a greater affinity for CD47 than the non-humanized antibody (see, e.g., Jones et al., Nature 321:522-25 (1986); Riechmann et al., Nature 332:323-27 (1988)).

Designing a humanized antibody may therefore include determining CDR loop conformations and structural determinants of the non-human variable regions, for example, by computer modeling, and then comparing the CDR loops and determinants to known human CDR loop structures and determinants (see, e.g., Padlan et al., FASEB 9:133-39 (1995); Chothia et al., Nature, 342:377-83 (1989)). Computer modeling may also be used to compare human structural templates selected by sequence homology with the non-human variable regions (see, e.g., Bajorath et al., Ther. Immunol. 2:95-103 (1995); EP-0578515-A3). If humanization of the non-human CDRs results in a decrease in binding affinity, computer modeling may aid in identifying specific amino acid residues that could be changed by site-directed or other mutagenesis techniques to partially, completely, or supra-optimally (i.e., increase to a level greater than that of the non-humanized antibody) restore affinity. Those having ordinary skill in the art are familiar with these techniques and will readily appreciate numerous variations and modifications to such design strategies.

One such method for preparing a humanized antibody is called veneering. Veneering framework (FR) residues refers to the selective replacement of FR residues from, e.g., a rodent heavy or light chain V region, with human FR residues in order to provide a xenogeneic molecule comprising an antigen-binding site that retains substantially all of the native FR polypeptide folding structure. Veneering techniques are based on the understanding that the ligand binding characteristics of an antigen-binding site are determined primarily by the structure and relative disposition of the heavy and light chain CDR sets within the antigen-binding surface (see, e.g., Davies et al., Ann. Rev. Biochem. 59:439-73, (1990)). Thus, antigen binding specificity can be preserved in a humanized antibody when the CDR structures, their interaction with each other, and their interaction with the rest of the V region domains are carefully maintained. By using veneering techniques, exterior (e.g., solvent-accessible) FR residues that are readily encountered by the immune system are selectively replaced with human residues to provide a hybrid molecule that comprises either a weakly immunogenic, or substantially non-immunogenic veneered surface.

The process of veneering makes use of the available sequence data for human antibody variable domains compiled by Kabat et al., in Sequences of Proteins of Immunological Interest, 4th ed., (U.S. Dept. of Health and Human Services, U.S. Government Printing Office, 1991), updates to the Kabat database, and other accessible U.S. and foreign databases (both nucleic acid and protein). Solvent accessibilities of V region amino acids can be deduced from the known three-dimensional structure for human and murine antibody fragments. Initially, the FR amino acid sequence of the variable domains of an antibody molecule of interest are compared with corresponding FR sequences of human variable domains obtained from the above-identified databases and publications. The most homologous human V regions are then compared residue by residue to corresponding murine amino acids. The residues in the murine FR that differ from the human counterpart are replaced by the residues present in the human moiety using recombinant techniques well known in the art. Residue switching is only carried out with moieties that are at least partially exposed (solvent accessible), and care is exercised in the replacement of amino acid residues that may have a significant effect on the tertiary structure of V region domains, such as proline, glycine, and charged amino acids.

In this manner, the resultant “veneered” antigen-binding sites are designed to retain the rodent CDR residues, the residues substantially adjacent to the CDRs, the residues identified as buried or mostly buried (solvent inaccessible), the residues believed to participate in non-covalent (e.g., electrostatic and hydrophobic) contacts between heavy and light chain domains, and the residues from conserved structural regions of the FRs that are believed to influence the “canonical” tertiary structures of the CDR loops (see, e.g., Chothia et al., Nature, 342:377-383 (1989)). These design criteria are then used to prepare recombinant nucleotide sequences that combine the CDRs of both the heavy and light chain of an antigen-binding site into human-appearing FRs that can be used to transfect mammalian cells for the expression of recombinant human antibodies that exhibit the antigen specificity of the rodent antibody molecule.

For particular uses, antigen-binding fragments of antibodies may be desired. Antibody fragments, F(ab′)₂, Fab, Fab′, Fv, and Fd, can be obtained, for example, by proteolytic hydrolysis of the antibody, for example, pepsin or papain digestion of whole antibodies according to conventional methods. As an illustration, antibody fragments can be produced by enzymatic cleavage of antibodies with pepsin to provide a fragment denoted F(ab′)₂. This fragment can be further cleaved using a thiol reducing agent to produce an Fab′ monovalent fragment. Optionally, the cleavage reaction can be performed using a blocking group for the sulfhydryl groups that result from cleavage of disulfide linkages. As an alternative, an enzymatic cleavage of an antibody using papain produces two monovalent Fab fragments and an Fc fragment (see, e.g., U.S. Pat. No. 4,331,647; Nisonoff et al., Arch. Biochem. Biophys. 89:230 (1960); Porter, Biochem. J. 73:119 (1959); Edelman et al., in Methods in Enzymology 1:422 (Academic Press 1967); Weir, Handbook of Experimental Immunology, Blackwell Scientific, Boston (1986)). The antigen binding fragments may be separated from the Fc fragments by affinity chromatography, for example, using immobilized protein A, protein G, an Fc specific antibody, or immobilized CD47 or CD47 extracellular domain polypeptide or a fragment thereof. Other methods for cleaving antibodies, such as separating heavy chains to form monovalent light-heavy chain fragments (Fd), further cleaving of fragments, or other enzymatic, chemical, or genetic techniques may also be used, so long as the fragments bind to the antigen that is recognized by the intact antibody.

An antibody fragment may also be any synthetic or genetically engineered protein that acts like an antibody in that it binds to a specific antigen to form a complex. For example, antibody fragments include isolated fragments consisting of the light chain variable region, Fv fragments consisting of the variable regions of the heavy and light chains, recombinant single chain polypeptide molecules in which light and heavy variable regions are connected by a peptide linker (scFv proteins), and minimal recognition units consisting of the amino acid residues that mimic the hypervariable region. The antibody of the present invention preferably comprises at least one variable region domain. The variable region domain may be of any size or amino acid composition and will generally comprise at least one hypervariable amino acid sequence responsible for antigen binding and which is adjacent to or in frame with one or more framework sequences. In general terms, the variable (V) region domain may be any suitable arrangement of immunoglobulin heavy (V_{H}) and/or light (V_{L}) chain variable domains. Thus, for example, the V region domain may be monomeric and be a V_{H} or V_{L} domain, which is capable of independently binding antigen with acceptable affinity. Alternatively, the V region domain may be dimeric and contain V_{H}-V_{H}, V_{H}-V_{L}, or V_{L}-V_{L}, dimers. Preferably, the V region dimer comprises at least one V_{H} and at least one V_{L} chain that are non-covalently associated (hereinafter referred to as Fv). If desired, the chains may be covalently coupled either directly, for example via a disulfide bond between the two variable domains, or through a linker, for example a peptide linker, to form a single chain Fv (scFv).

A minimal recognition unit is an antibody fragment comprising a single complementarity-determining region (CDR). Such CDR peptides can be obtained by constructing polynucleotides that encode the CDR of an antibody of interest. The polynucleotides are prepared, for example, by using the polymerase chain reaction to synthesize the variable region using mRNA isolated from or contained within antibody-producing cells as a template according to methods practiced by persons skilled in the art (see, for example, Larrick et al., Methods: A Companion to Methods in Enzymology 2:106, (1991); Courtenay-Luck, “Genetic Manipulation of Monoclonal Antibodies,” in Monoclonal Antibodies. Production, Engineering and Clinical Application, Ritter et al. (eds.), page 166 (Cambridge University Press 1995); and Ward et al., “Genetic Manipulation and Expression of Antibodies,” in Monoclonal Antibodies. Principles and Applications, Birch et al., (eds.), page 137 (Wiley-Liss, Inc. 1995)). Alternatively, such CDR peptides and other antibody fragment can be synthesized using an automated peptide synthesizer.

According to certain embodiments, non-human, human, or humanized heavy chain and light chain variable regions of any of the Ig molecules described herein may be constructed as scFv polypeptide fragments (single chain antibodies). See, e.g., Bird et al., Science 242:423-426 (1988); Huston et al., Proc. Natl. Acad. Sci. USA 85:5879-83 (1988)). Multi-functional scFv fusion proteins may be generated by linking a polynucleotide sequence encoding an scFv polypeptide in-frame with at least one polynucleotide sequence encoding any of a variety of known effector proteins. These methods are known in the art, and are disclosed, for example, in EP-B1-0318554, U.S. Pat. No. 5,132,405, U.S. Pat. No. 5,091,513, and U.S. Pat. No. 5,476,786. By way of example, effector proteins may include immunoglobulin constant region sequences. See, e.g., Hollenbaugh et al., J. Immunol. Methods 188:1-7 (1995). Other examples of effector proteins are enzymes. As a non-limiting example, such an enzyme may provide a biological activity for therapeutic purposes (see, e.g., Siemers et al., Bioconjug. Chem. 8:510-19 (1997)), or may provide a detectable activity, such as horseradish peroxidase-catalyzed conversion of any of a number of well-known substrates into a detectable product, for diagnostic uses.

The scFv may, in certain embodiments, be fused to peptide or polypeptide domains that permit detection of specific binding between the fusion protein and antigen. For example, the fusion polypeptide domain may be an affinity tag polypeptide. Binding of the scFv fusion protein to a binding partner (e.g., a CD47 extracellular domain) may therefore be detected using an affinity polypeptide or peptide tag, such as an avidin, streptavidin or a His (e.g., polyhistidine) tag, by any of a variety of techniques with which those skilled in the art will be familiar. Detection techniques may also include, for example, binding of an avidin or streptavidin fusion protein to biotin or to a biotin mimetic sequence (see, e.g., Luo et al., J. Biotechnol. 65:225 (1998) and references cited therein), direct covalent modification of a fusion protein with a detectable moiety (e.g., a labeling moiety), non-covalent binding of the fusion protein to a specific labeled reporter molecule, enzymatic modification of a detectable substrate by a fusion protein that includes a portion having enzyme activity, or immobilization (covalent or non-covalent) of the fusion protein on a solid-phase support. An scFv fusion protein comprising a CD47-specific immunoglobulin-derived polypeptide may be fused to another polypeptide such as an effector peptide having desirable affinity properties (see, e.g., U.S. Pat. No. 5,100,788; WO 89/03422; U.S. Pat. No. 5,489,528; U.S. Pat. No. 5,672,691; WO 93/24631; U.S. Pat. No. 5,168,049; U.S. Pat. No. 5,272,254; EP 511,747). As provided herein, scFv polypeptide sequences may be fused to fusion polypeptide sequences, including effector protein sequences, that may include full-length fusion polypeptides and that may alternatively contain variants or fragments thereof. An scFv fusion protein comprising a CD47 ligand specific immunoglobulin derived polypeptide may be similarly prepared.

Antibodies may also be identified and isolated from human immunoglobulin phage libraries, from rabbit immunoglobulin phage libraries, from mouse immunoglobulin phage libraries, and/or from chicken immunoglobulin phage libraries (see, e.g., Winter et al., Annu. Rev. Immunol. 12:433-55 (1994); Burton et al., Adv. Immunol. 57:191-280 (1994); U.S. Pat. No. 5,223,409; Huse et al., Science 246:1275-81 (1989); Schlebusch et al., Hybridoma 16:47-52 (1997) and references cited therein; Rader et al., J. Biol. Chem. 275:13668-76 (2000); Popkov et al., J. Mol. Biol. 325:325-35 (2003); Andris-Widhopf et al., J. Immunol. Methods 242:159-31 (2000)). Antibodies isolated from non-human species or non-human immunoglobulin libraries may be genetically engineered according to methods described herein and known in the art to “humanize” the antibody or fragment thereof. Immunoglobulin variable region gene combinatorial libraries may be created in phage vectors that can be screened to select Ig fragments (Fab, Fv, scFv, or multimers thereof) that bind specifically to CD47 as described herein (see, e.g., U.S. Pat. No. 5,223,409; Huse et al., Science 246:1275-81 (1989); Sastry et al., Proc. Natl. Acad. Sci. USA 86:5728-32 (1989); Alting-Mees et al., Strategies in Molecular Biology 3:1-9 (1990); Kang et al., Proc. Natl. Acad. Sci. USA 88:4363-66 (1991); Hoogenboom et al., J. Molec. Biol. 227:381-388 (1992); Schlebusch et al., Hybridoma 16:47-52 (1997) and references cited therein; U.S. Pat. No. 6,703,015).

For example, a library containing a plurality of polynucleotide sequences encoding Ig variable region fragments may be inserted into the genome of a filamentous bacteriophage, such as M13 or a variant thereof, in frame with the sequence encoding a phage coat protein such as gene III or gene VIII. A fusion protein may be a fusion of the coat protein with the light chain variable region domain and/or with the heavy chain variable region domain. According to certain embodiments, immunoglobulin Fab fragments may also be displayed on a phage particle (see, e.g., U.S. Pat. No. 5,698,426).

Heavy and light chain immunoglobulin cDNA expression libraries may also be prepared in lambda phage, for example, using λImmunoZap™(H) and λImmunoZap™(L) vectors (Stratagene, La Jolla, Calif.). Briefly, mRNA is isolated from a B cell population and used to create heavy and light chain immunoglobulin cDNA expression libraries in the λImmunoZap(H) and λImmunoZap(L) vectors. These vectors may be screened individually or co-expressed to form Fab fragments or antibodies (see Huse et al., supra; see also Sastry et al., supra). Positive plaques may subsequently be converted to a non-lytic plasmid that allows high-level expression of monoclonal antibody fragments from E. coli.

Phage that display an Ig fragment (e.g., an Ig V-region or Fab) that binds to CD47, and the CD47 extracellular domain in particular, may be selected by mixing the phage library with CD47 or the CD47 extracellular domain or a fragment thereof, or by contacting the phage library with such polypeptide or peptide molecules immobilized on a solid matrix under conditions and for a time sufficient to allow binding. Unbound phage are removed by a wash, and specifically bound phage (i.e., phage that contain an CD47 extracellular domain-specific Ig fragment) are then eluted (see, e.g., Messmer et al., Biotechniques 30:798-802 (2001)). Eluted phage may be propagated in an appropriate bacterial host, and generally, successive rounds of binding to CD47 or an CD47 extracellular domain and elution can be repeated to increase the yield of phage expressing the CD47-specific immunoglobulin. Such methods may also be used to identify phage that express a CD47 ligand specific immunoglobulin.

Phage display techniques may also be used to select Ig fragments or single chain antibodies that bind to the CD47 and the CD47 extracellular domain. For examples of suitable vectors having multicloning sites into which candidate nucleic acid molecules (e.g., DNA) encoding such antibody fragments or related peptides may be inserted, see, e.g., McLafferty et al., Gene 128:29-36 (1993); Scott et al., Science 249:386-90 (1990); Smith et al., Meth. Enzymol. 217:228-57 (1993); Fisch et al., Proc. Natl. Acad. Sci. USA 93:7761-66 (1996)). The inserted DNA molecules may comprise randomly generated sequences, or may encode variants of a known peptide or polypeptide domain (such as a CD47 ligand) that specifically binds to CD47. Generally, the nucleic acid insert encodes a peptide of up to 60 amino acids, or may encode a peptide of 3 to 35 amino acids, or may encode a peptide of 6 to 20 amino acids. The peptide encoded by the inserted sequence is displayed on the surface of the bacteriophage. Phage expressing a binding domain for CD47 may be selected on the basis of specific binding to an immobilized CD47 or CD47 extracellular domain or a fragment thereof. Well-known recombinant genetic techniques may be used to construct fusion proteins containing the fragment. For example, a polypeptide may be generated that comprises a tandem array of two or more similar or dissimilar affinity selected CD47 binding peptide domains, in order to maximize binding affinity for CD47 of the resulting product. Such methods may also be used to select Ig fragments or single chain antibodies that bind to a CD47 ligand.

Combinatorial mutagenesis strategies using phage libraries may also be used for humanizing non-human variable regions (see, e.g., Rosok et al., J. Biol. Chem. 271:22611-18 (1996); Rader et al., Proc. Natl. Acad. Sci. USA 95:8910-15 (1998)). Humanized variable regions that have binding affinity that is minimally reduced or that is comparable to the non-human variable region can be selected, and the nucleotide sequences encoding the humanized variable regions may be determined by standard techniques (see, Sambrook et al., Molecular Cloning: A Laboratory Manual, Cold Spring Harbor Press (2001)). The affinity selected Ig-encoding sequence may then be cloned into another suitable vector for expression of the Ig fragment or, optionally, may be cloned into a vector containing Ig constant regions, for expression of whole immunoglobulin chains.

Similarly, portions or fragments, such as Fab and Fv fragments, of CD47-specific antibodies may be constructed using conventional enzymatic digestion or recombinant DNA techniques to incorporate the variable regions of a gene that encodes an antibody specific for CD47 and in particular embodiments, for the CD47 extracellular domain. Within one embodiment, in a hybridoma the variable regions of a gene expressing a monoclonal antibody of interest are amplified using nucleotide primers. These primers may be synthesized by one of ordinary skill in the art, or may be purchased from commercially available sources (see, e.g., Stratagene (La Jolla, Calif.), which sells primers for amplifying mouse and human variable regions. The primers may be used to amplify heavy or light chain variable regions, which may then be inserted into vectors such as ImmunoZAP™ H or ImmunoZAP™ L (Stratagene), respectively. These vectors may then be introduced into E. coli, yeast, or mammalian-based systems for expression. Large amounts of a single-chain protein containing a fusion of the V_{H} and V_{L} domains may be produced using these methods (see Bird et al., Science 242:423-426 (1988)). In addition, such techniques may be used to humanize a non-human antibody V region without altering the binding specificity of the antibody. Such methods may also be used to make fragments of antibodies that bind to a CD47 ligand.

In certain other embodiments, specific antibodies are multimeric antibody fragments. Useful methodologies are described generally, for example in Hayden et al., Curr Opin. Immunol. 9:201-12 (1997) and Coloma et al., Nat. Biotechnol. 15:159-63 (1997). For example, multimeric antibody fragments may be created by phage techniques to form miniantibodies (U.S. Pat. No. 5,910,573) or diabodies (Holliger et al., Cancer Immunol. Immunother. 45:128-30 (1997)). Multimeric fragments may be generated that are multimers of a specific Fv.

Multimeric antibodies include bispecific and bifunctional antibodies comprising a first Fv specific for an antigen associated with a second Fv having a different antigen specificity (see, e.g., Drakeman et al., Expert Opin. Investig. Drugs 6:1169-78 (1997); Koelemij et al., J. Immunother. 22:514-24 (1999); Marvin et al., Acta Pharmacol. Sin. 26:649-58 (2005); Das et al., Methods Mol. Med. 109:329-46 (2005)). For example, in one embodiment, a bispecific antibody comprises an Fv, or other antigen-binding fragment described herein, that specifically binds to the antigen and comprises an Fv, or other antigen-binding fragment, that specifically binds to another cell surface polypeptide, for example, a cell surface antigen that when bound by a specific antibody contributes to, facilitates, or is capable of altering (suppressing or enhancing) immunoresponsiveness of an immune cell.

Introducing amino acid mutations into immunoglobulin molecules may be useful to increase the specificity or affinity of the immunoglobulin for the specific antigen, or to alter an effector function. Immunoglobulins exhibiting higher affinity for the antigen may be generated by site-directed mutagenesis of particular residues. Computer assisted three-dimensional molecular modeling may be used to identify the amino acid residues to be changed in order to improve affinity for the antigen (see, e.g., Mountain et al., Biotechnol. Genet. Eng. Rev. 10:1-142 (1992)). Alternatively, combinatorial libraries of CDRs may be generated in M13 phage and screened for immunoglobulin fragments with improved affinity (see, e.g., Glaser et al., J. Immunol. 149:3903-13 (1992); Barbas et al., Proc. Natl. Acad. Sci. USA 91:3809-13 (1994); U.S. Pat. No. 5,792,456).

In certain embodiments, the antibody may be genetically engineered to have an altered effector function. For example, the antibody may have an altered capability (increased or decreased in a biologically or statistically significant manner) to mediate complement dependent cytotoxicity (CDC) or antibody dependent cellular cytotoxicity (ADCC) or an altered capability for binding to effector cells via Fc receptors present on the effector cells. Effector functions may be altered by site-directed mutagenesis (see, e.g., Duncan et al., Nature 332:563-64 (1988); Morgan et al., Immunology 86:319-24 (1995); Eghtedarzedeh-Kondri et al., Biotechniques 23:830-34 (1997)). For example, mutation of the glycosylation site on the Fc portion of the immunoglobulin may alter the capability of the immunoglobulin to fix complement (see, e.g., Wright et al., Trends Biotechnol. 15:26-32 (1997)). Other mutations in the constant region domains may alter the ability of the immunoglobulin to fix complement or to effect ADCC (see, e.g., Duncan et al., Nature 332:563-64 (1988); Morgan et al., Immunology 86:319-24 (1995); Sensel et al., Mol. Immunol. 34:1019-29 (1997)). (See also, e.g., U.S. Patent Publication Nos. 2003/0118592; 2003/0133939).

The nucleic acid molecules encoding an antibody or fragment thereof that specifically binds CD47, as described herein, may be propagated and expressed according to any of a variety of well-known procedures for nucleic acid excision, ligation, transformation, and transfection. Thus, in certain embodiments expression of an antibody fragment may be preferred in a prokaryotic host cell, such as Escherichia coli (see, e.g., Pluckthun et al., Methods Enzymol. 178:497-515 (1989)). In certain other embodiments, expression of the antibody or an antigen-binding fragment thereof may be preferred in a eukaryotic host cell, including yeast (e.g., Saccharomyces cerevisiae, Schizosaccharomyces pombe, and Pichia pastoris); animal cells (including mammalian cells); or plant cells. Examples of suitable animal cells include, but are not limited to, myeloma, COS, CHO, or hybridoma cells. Examples of plant cells include tobacco, corn, soybean, and rice cells. By methods known to those having ordinary skill in the art and based on the present disclosure, a nucleic acid vector may be designed for expressing foreign sequences in a particular host system, and then polynucleotide sequences encoding the CD47-binding antibody (or fragment thereof) may be inserted. The regulatory elements will vary according to the particular host. Similarly, nucleic acid molecules encoding an antibody or fragment thereof that specifically binds a CD47 ligand may be propagated and expressed according to any of a variety of well-known procedures for nucleic acid excision, ligation, transformation, and transfection.

One or more replicable expression vectors containing a polynucleotide encoding a variable and/or constant region may be prepared and used to transform an appropriate cell line, for example, a non-producing myeloma cell line, such as a mouse NSO line or a bacteria, such as E. coli, in which production of the antibody will occur. In order to obtain efficient transcription and translation, the polynucleotide sequence in each vector should include appropriate regulatory sequences, particularly a promoter and leader sequence operatively linked to the variable domain sequence. Particular methods for producing antibodies in this way are generally well known and routinely used. For example, molecular biology procedures are described by Sambrook et al. (Molecular Cloning, A Laboratory Manual, 2nd ed., Cold Spring Harbor Laboratory, New York, 1989; see also Sambrook et al., 3rd ed., Cold Spring Harbor Laboratory, New York, (2001)). DNA sequencing can be performed as described in Sanger et al. (Proc. Natl. Acad. Sci. USA 74:5463 (1977)) and the Amersham International plc sequencing handbook and including improvements thereto.

Site directed mutagenesis of an immunoglobulin variable (V region), framework region, and/or constant region may be performed according to any one of numerous methods described herein and practiced in the art (Kramer et al., Nucleic Acids Res. 12:9441 (1984); Kunkel Proc. Natl. Acad. Sci. USA 82:488-92 (1985); Kunkel et al., Methods Enzymol. 154:367-82 (1987)). Random mutagenesis methods to identify residues of the antibody that are either important to binding to the antigen or that, when changed, do not alter binding of the antigen to the antibody can also be performed according to procedures that are routinely practiced by a person skilled in the art (e.g., alanine scanning mutagenesis; error prone polymerase chain reaction mutagenesis; and oligonucleotide-directed mutagenesis (see, e.g., Sambrook et al. Molecular Cloning. A Laboratory Manual, 3rd ed., Cold Spring Harbor Laboratory Press, NY (2001))). Additionally, numerous publications describe techniques suitable for the preparation of antibodies by manipulation of DNA, creation of expression vectors, and transformation of appropriate cells (Mountain et al., in Biotechnology and Genetic Engineering Reviews (ed. Tombs, M P, 10, Chapter 1, Intercept, Andover, UK (1992)); International Patent Publication No. WO 91/09967).

The antibodies and antigen-binding fragments thereof that specifically bind to CD47, including antibodies that specifically bind to the CD47 extracellular domain, may also be useful as reagents for immunochemical analyses to detect the presence of CD47, or a fragment thereof, in a biological sample. The following methods may also be adapated for detecting the presence of a CD47 ligand. In certain embodiments, an antibody that specifically binds to the CD47 extracellular domain may be used to detect expression of CD47. In certain particular embodiments, one antibody or a panel of antibodies may be exposed to cells that express CD47, and expression of CD47 may be determined by detection using another CD47-specific antibody that binds to a different epitope than the antibody or antibodies initially permitted to interact with the cells.

For such a purpose CD47-binding immunoglobulin (or fragment thereof) as described herein may contain a detectable moiety or label such as an enzyme, cytotoxic agent, or other reporter molecule, including a dye, radionuclide, luminescent group, fluorescent group, or biotin, or the like. The CD47-specific immunoglobulin or fragment thereof may be radiolabeled for diagnostic or therapeutic applications. Techniques for radiolabeling of antibodies are known in the art (see, e.g., Adams, In Vivo 12:11-21 (1998); Hiltunen, Acta Oncol. 32:831-9 (1993)). The effector or reporter molecules may be attached to the antibody through any available amino acid side-chain, terminal amino acid, or carbohydrate functional group located in the antibody, provided that the attachment or attachment process does not adversely affect the binding properties such that the usefulness of the molecule is abrogated. Particular functional groups include, for example, any free amino, imino, thiol, hydroxyl, carboxyl, or aldehyde group. Attachment of the antibody or antigen-binding fragment thereof and the effector and/or reporter molecule(s) may be achieved via such groups and an appropriate functional group in the effector or reporter molecule. The linkage may be direct or indirect through spacing or bridging groups (see, e.g., International Patent Application Publication Nos. WO 93/06231, WO 92/22583, WO 90/091195, and WO 89/01476; see also, e.g., commercial vendors such as Pierce Biotechnology, Rockford, Ill.).

As provided herein and according to methodologies well known in the art, polyclonal and monoclonal antibodies may be used for the affinity isolation of CD47 and fragments thereof (see, e.g., Hermanson et al., Immobilized Affinity Ligand Techniques, Academic Press, Inc. New York, (1992)). Briefly, an antibody (or antigen-binding fragment thereof) may be immobilized on a solid support material, which is then contacted with a sample that contains CD47. The sample interacts with the immobilized antibody under conditions and for a time that are sufficient to permit binding of CD47 to the immobilized antibody; non-binding components (that is, those components unrelated to CD47) of the sample are removed; and then CD47 is released from the immobilized antibody using an appropriate eluting solution.

In certain embodiments, anti-idiotype antibodies that recognize and bind specifically to an antibody (or antigen-binding fragment thereof) that specifically binds to CD47, including an antibody that specifically binds to the CD47 extracellular domain, are provided, and methods for using these anti-idiotype antibodies are also provided. Anti-idiotype antibodies may be generated as polyclonal antibodies or as monoclonal antibodies by the methods described herein, using an anti-CD47 extracellular domain antibody (or antigen-binding fragment thereof) as immunogen. Anti-idiotype antibodies or fragments thereof may also be generated by any of the recombinant genetic engineering methods described above or by phage display selection. Anti-idiotype antibodies may be further engineered to provide a chimeric or humanized anti-idiotype antibody, according to the description provided in detail herein. An anti-idiotype antibody may bind specifically to the antigen-binding site of the anti-CD47 extracellular domain antibody such that binding of the antibody to the CD47 extracellular domain extracellular domain is competitively inhibited. Alternatively, an anti-idiotype antibody as provided herein may not competitively inhibit binding of an anti-CD47 extracellular domain antibody to the CD47 extracellular domain.

In one embodiment, an anti-idiotype antibody may be used to alter the immunoresponsiveness of an immune cell. In certain embodiments, an anti-idiotype antibody may be used to suppress the immunoresponsiveness of an immune cell and to treat an immunological disease or disorder. An anti-idiotype antibody specifically binds to an antibody that specifically binds to the CD47 extracellular domain, and the antigen-binding site of the anti-idiotype antibody mimics the epitope of the CD47 extracellular domain, that is, the anti-idiotype antibody will bind to cognate ligands as well as antibodies that specifically bind to the CD47. Thus, an anti-idiotype antibody may be useful for preventing, blocking, or reducing binding of a cognate ligand that when such ligand binds to CD47, it alters (i.e., increases or decreases in a statistically or biologically significant manner) the immunoresponsiveness of an immune cell.

Anti-idiotype antibodies are also useful for immunoassays to determine the presence of anti-CD47 antibodies in a biological sample. For example, immunoassays, such as an ELISA and other assays described herein that are practiced by persons skilled in the art, may be used to determine the presence of an immune response induced by administering (i.e., immunizing) a host with a fusion polypeptide comprising the extracellular domain of CD47 (or a variant thereof) as described herein.

### Methods for Determining the Effects of Extracellular Domain CD47-Fusion Polypeptides and of Other Agents That Specifically Bind to CD47

Binding of a fusion polypeptide comprising the extracellular domain of a CD47 (or variant thereof) fused to a heterologous polypeptide moiety, such as a Fc polypeptide (or variant thereof), alters at least one biological function of CD47 that is expressed by a cell. Also as described herein, the interaction between the fusion polypeptide and a CD47 ligand secreted by a cell or expressed on the cell surface of an immune cell may alter (i.e., suppresses or enhances) the immunoresponsiveness of the cell. Alteration of the immunoresponsiveness of an immune cell may also be effected by a bioactive agent (compound or molecule) in a manner similar to a fusion polypeptide comprising the extracellular domain of CD47. Bioactive agents include, for example, small molecules, nucleic acids (such as aptamers), antibodies and fragments thereof (which are discussed in detail herein), and peptide fusion proteins (such as peptide-Fc fusion proteins). An agent may interact with and bind to the extracellular domain of CD47 at a location or binding site of CD47 that is the same location or proximal to the same location as where a CD47 ligand binds. In addition, the agent described herein that specifically binds to CD47 may inhibit binding of a viral CD47-like polypeptide to the CD47 ligand. Alternatively, alteration of immunoresponsiveness by an agent in a manner similar to the effect of a fusion polypeptide comprising a CD47 extracellular domain, or variant thereof, may result from binding or interaction of the agent with the CD47 ligand at the same location or at a location distal from that at which the fusion polypeptide binds. An agent that specifically binds to a CD47 ligand includes an antibody, or antigen binding fragment thereof, that specifically binds to the CD47 ligand, such as an antibody that specifically binds to SIRPα. Binding studies, including competitive binding assays, and functional assays, which indicate the level of immunoresponsiveness of a cell, may be performed according to methods described herein and practiced in the art to determine and compare the capability and level with which an agent binds to and affects the immunoresponsiveness of an immune cell.

Methods are provided herein for identifying an agent that alters (e.g., suppresses or enhances in a statistically or biologically significant manner) immunoresponsiveness of an immune cell and for characterizing and determining the level of suppression or enhancement of such an agent once identified. Such methods, which are discussed in greater detail herein and are familiar to persons skilled in the art, include but are not limited to, binding assays, such as immunoassays (e.g., ELISA, radioimmunoassay, immunoblot, etc.), competitive binding assays, and surface plasmon resonance. These methods comprise contacting (mixing, combining with, or in some manner permitting interaction among) (1) a candidate agent; (2) a viral CD47-like polypeptide (a number of which are described in detail herein and which may also include the extracellular domain of a viral CD47-like polypeptide, or a fusion polypeptide comprising the extracellular domain of a viral CD47-like polypeptide); and (3) a CD47 ligand (for example, SIRP-α, SIRP-beta 2, thrombospondin-1, αᵥβ₃ integrin, and α₂β₁ integrin), under conditions and for a time sufficient to permit interaction between the CD47 ligand and a viral CD47-like polypeptide. Such exemplary methods and techniques may also be used to characterize the CD47 fusion polypeptides described herein. Accordingly, the methods described herein, which refer to a candidate agent, also may be used when the candidate agent is any one of the fusion polypeptides described herein.

Conditions for a particular assay include temperature, buffers (including salts, cations, media), and other components that maintain the integrity of the candidate agent (or CD47 fusion polypeptide); viral CD47-like polypeptide; and CD47 ligand, with which a person skilled in the art will be familiar and/or which can be readily determined. The interaction, or level of binding, of the viral CD47-like polypeptide to the CD47 ligand in the presence of the candidate agent may be determined and compared to a level of binding of the viral CD47-like polypeptide to the CD47 ligand in the absence of the candidate agent. A decrease in the level of binding of the viral CD47-like polypeptide to the CD47 ligand in the presence of the candidate agent indicates that the candidate agent inhibits binding of the viral CD47-like polypeptide to the CD47 ligand. The candidate agent is then contacted (mixed, combined with, or in some manner permitted to interaction) with a CD47 ligand and an immune cell that expresses CD47, under conditions and for a time sufficient to permit interaction between a CD47 ligand and CD47. The level of binding between the CD47 ligand and the immune cell in the presence of the candidate agent is compared with the level of binding of the CD47 ligand to the immune cell in the absence of the candidate agent. A decrease in the level of binding of the CD47 ligand to the immune cell expressing CD47 in the presence of the candidate agent indicates that the candidate agent alters the immunoresponsiveness of the immune cell. In a specific embodiment of the method, the candidate agent suppresses immunoresponsiveness of the immune cell.

In another embodiment, a method for identifying an agent that alters (suppresses or enhances) immunoresponsiveness of an immune cell comprises determining the level of immunoresponsiveness of an immune cell that expresses CD47 in the presence of the agent. In certain specific embodiments, an agent is identified that suppresses immunoresponsiveness of an immune cell. Immunoresponsiveness may be determined according to methods practiced in the art such as measuring levels of cytokines, cell maturation (e.g., maturation of dendritic cells), proliferation, and stimulation. Immunoresponsiveness of an immune cell may also be determined by evaluating changes in cell adhesion and cell migration and by examining expression, cellular location, and post-translational modification of cellular proteins, such as determining the tyrosine phosphorylation pattern of cellular proteins, including but not limited to cytoskeletal proteins and other proteins that affect cell adhesion and migration.

Numerous assays and techniques are practiced by persons skilled in the art for determining the interaction between, or binding between, a biological molecule and a cognate ligand. Accordingly, interaction between a CD47 ligand and CD47, including the effect of a bioactive agent on this interaction and/or binding in the presence of the agent, can be readily determined by such assays and techniques as described in detail herein and routinely practiced by persons skilled in the art.

Appropriate conditions for permitting interaction of the reaction components according to this method and other methods described herein include, for example, appropriate concentrations of reagents and components (including a CD47 ligand, the candidate agent, an immune cell that expresses CD47, and/or a viral CD47-like polypeptide, or fragment or extracellular domain thereof (or fusion polypeptide comprising a viral CD47-like extracellular domain), temperature, and buffers with which a skilled person will be familiar. Concentrations of reaction components, buffers, temperature, and time period sufficient to permit interaction of the reaction components can be determined and/or adjusted according to methods described herein and with which persons skilled in the art are familiar. To practice the methods described herein, a person skilled in the art will also readily appreciate and understand which controls are appropriately included when practicing these methods.

Numerous assays and techniques are practiced by persons skilled in the art for determining the interaction between or binding between a biological molecule and a cognate ligand. Accordingly, interaction between a CD47 ligand and a viral CD47-like polypeptide and interaction between an immune cell expressing CD47 and a CD47 ligand, including the effect of a bioactive agent on this interaction and/or binding in the presence of the agent can be readily determined by such assays and techniques, which may include a competitive assay format. Exemplary methods include but are not limited to fluorescence resonance energy transfer, fluorescence polarization, time-resolved fluorescence resonance energy transfer, scintillation proximity assays, reporter gene assays, fluorescence quenched enzyme substrate, chromogenic enzyme substrate and electrochemiluminescence, immunoassays, (such as enzyme-linked immunosorbant assays (ELISA), radioimmunoassay, immunoblotting, immunohistochemistry, and the like), surface plasmon resonance, cell-based assays such as those that use reporter genes, and functional assays (e.g., assays that measure immune function and immunoresponsiveness). Many of the methods described herein and known to those skilled in the art may be adapted to high throughput screening for analyzing large numbers of bioactive agents such as from libraries of compounds to determine the effect of an agent on the binding, interaction, or biological function of CD47 and a CD47 ligand and the effect of an agent on immunoresponsiveness of an immune cell (see, e.g., High Throughput Screening. The Discovery of Bioactive Substances, Devlin, ed., (Marcel Dekker New York, 1997)).

The techniques and assay formats may also include secondary reagents, such as specific antibodies, that are useful for detecting and/or amplifying a signal that indicates formation of a complex, such as between a CD47 ligand and a viral CD47-like polypeptide (or extracellular domain thereof or fusion polypeptide comprising the extracellular domain), or such as between an immune cell expressing CD47 and a CD47 ligand. One or more of the assay components or secondary reagents may be attached to a detectable moiety (or label or reporter molecule) such as an enzyme, cytotoxicity agent, or other reporter molecule, including a dye, radionuclide, luminescent group, fluorescent group, or biotin, or the like. Techniques for radiolabeling of antibodies and other polypeptides are known in the art (see, e.g., Adams, In Vivo 12:11-21 (1998); Hiltunen, Acta Oncol. 32:831-9 (1993)). The detectable moiety may be attached to a polypeptide (e.g., an antibody), such as through any available amino acid side-chain, terminal amino acid, or carbohydrate functional group located in the polypeptide, provided that the attachment or attachment process does not adversely affect the binding properties such that the usefulness of the molecule is abrogated. Particular functional groups include, for example, any free amino, imino, thiol, hydroxyl, carboxyl, or aldehyde group. Attachment of the polypeptide and the detectable moiety may be achieved via such groups and an appropriate functional group in the detectable moiety. The linkage may be direct or indirect through spacing or bridging groups (see, e.g., International Patent Application Publication Nos. WO 93/06231, WO 92/22583, WO 90/091195, and WO 89/01476; see also, e.g., commercial vendors such as Pierce Biotechnology, Rockford, Ill.).

The immune cell may be present in or isolated from a biological sample as described herein. For example, the immune cell may be obtained from a primary or long-term cell culture or may be present in or isolated from a biological sample obtained from a subject (human or non-human animal).

Methods are also provided and described herein for determining the effect of a CD47 Fc fusion polypeptide (or an agent that acts in a similar manner as a CD47 Fc fusion polypeptide, for example, an anti-CD47 ligand antibody) on Fc-mediated activation of an immune cell, for example, determining the capability of a fusion polypeptide to inhibit cytokine or chemokine production of an immune cell. Methods are also provided for determining the effect of CD47 Fc fusion polypeptide (or an agent) on immune complex induced cytokine production or chemokine production. Exemplary methods are provided in the examples.

A “biological sample” as used herein refers in certain embodiments to a sample containing at least one CD47 ligand or a CD47 polypeptide or variant or fragment (e.g., the extracellular domain) thereof. A biological sample may also contain a viral CD47-like polypeptide or variant or fragment (e.g., the extracellular domain) thereof. A biological sample may be a blood sample (from which serum or plasma may be prepared), biopsy specimen, body fluids (e.g., lung lavage, ascites, mucosal washings, synovial fluid), bone marrow, lymph nodes, tissue explant, organ culture, or any other tissue or cell preparation from a subject or a biological source. A sample may further refer to a tissue or cell preparation in which the morphological integrity or physical state has been disrupted, for example, by dissection, dissociation, solubilization, fractionation, homogenization, biochemical or chemical extraction, pulverization, lyophilization, sonication, or any other means for processing a sample derived from a subject or biological source. The subject or biological source may be a human or non-human animal, a primary cell culture (e.g., immune cells, virus infected cells), or culture adapted cell line, including but not limited to, genetically engineered cell lines that may contain chromosomally integrated or episomal recombinant nucleic acid sequences, immortalized or immortalizable cell lines, somatic cell hybrid cell lines, differentiated or differentiatable cell lines, transformed cell lines, and the like.

The capability of a fusion polypeptide comprising a CD47 extracellular domain or variant thereof as described herein, and of an agent (e.g., an antibody or antigen-binding fragment thereof that specifically binds to CD47; an aptamer; peptide-IgFc fusion polypeptide; an antibody or antigen-binding fragment thereof that specifically binds to a CD47 ligand) described herein to suppress immunoresponsiveness of an immune cell and thus be useful for treating an immunological disease or disorder, such as an autoimmune disease or inflammatory disease or disorder, cardiovascular disease or disorder, a metabolic disease or disorder, or a proliferative disease or disorder, may be determined and evaluated in any one of a number of animal models described herein and used by persons skilled in the art (see, e.g., reviews by Taneja et al., Nat. Immunol. 2:781-84 (2001); Lam-Tse et al., Springer Semin. Immunopathol. 24:297-321 (2002)). For example, mice that have three genes, Tyro3, Mer, and Axl that encode receptor tyrosine kinases, knocked out exhibit several symptoms of autoimmune diseases, including rheumatoid arthritis and SLE (Lu et al., Science 293:228-29 (2001)). A murine model of spontaneous lupus-like disease has been described using NZB/WF1 hybrid mice (see, e.g., Drake et al., Immunol. Rev. 144:51-74 (1995)). An animal model for type I diabetes that permits testing of agents and molecules that affect onset, modulation, and/or protection of the animal from disease uses MHC transgenic (Tg) mice. Mice that express the HLA-DQ8 transgene (HLA-DQ8 is the predominant predisposing gene in human type I diabetes) and the HLA-DQ6 transgene (which is diabetes protective) were crossed with RIP(rat insulin promoter). B7-1-Tg mice to provide HLA-DQ8 RIP.B7-1 transgenic mice that develop spontaneous diabetes (see Wakeland et al., Curr. Opin. Immunol. 11:701-707 (1999); Wen et al., J. Exp. Med. 191:97-104 (2000)). (See also Brondum et al., Horm. Metab. Res. 37 Suppl 1:56-60 (2005)).

Animal models that may be used for characterizing the fusion polypeptide described herein and agents that are useful for treating immunological diseases and disorders, such as rheumatoid arthritis include a collagen-induced arthritis model (see, e.g., Kakimoto, Chin. Med. Sci. J. 6:78-83 (1991); Myers et al., Life Sci. 61:1861-78 (1997)) and an anti-collagen antibody-induced arthritis model (or collagen antibody induced arthritis (CAIA) (see, e.g., Kakimoto, supra; Wallace et al., J. Immunol. 162:5547 (1999)). Other applicable animal models for immunological diseases include an experimental autoimmune encephalomyelitis model (also called experimental allergic encephalomyelitis model), an animal model of multiple sclerosis; a psoriasis model that uses AGR129 mice that are deficient in type I and type II interferon receptors and deficient for the recombination activating gene 2 (Zenz et al., Nature 437:369-75 (2005); Boyman et al., J. Exp. Med. 199:731-36 (2004); published online Feb. 23, 2004); and a TNBS (2,4,6-trinitrobenzene sulphonic acid) mouse model for inflammatory bowel disease. Numerous animal models for cardiovascular disease are available and include models described in van Vlijmen et al., J. Clin. Invest. 93:1403-10 (1994); Kiriazis et al., Annu. Rev. Physiol. 62:321-51 (2000); Babu et al., Methods Mol. Med. 112:365-77 (2005).

Small Molecules

Bioactive agents may also include natural and synthetic molecules, for example, small molecules that bind to CD47, a viral CD47-like polypeptide, or to a CD47 ligand, and/or to a complex between CD47 and a CD47 ligand or between the viral CD47-like polypeptide and a CD47 ligand. Candidate agents for use in a method of screening for and identifying an agent that alters (suppresses or enhances) immunoresponsiveness of an immune cell and/or that inhibits binding of a CD47 ligand to CD47, may be provided as “libraries” or collections of compounds, compositions, or molecules.

Such molecules typically include compounds known in the art as “small molecules” and have molecular weights less than 10⁵ daltons, less than 10⁴ daltons, or less than 10³ daltons. For example, members of a library of test compounds can be administered to (combined with, added to) a plurality of samples, each containing at least one CD47 ligand as provided herein, and then the samples are assayed for their capability to enhance or inhibit binding or interaction of a viral CD47-like polypeptide to the ligand and/or to enhance or inhibit binding of the at least one CD47 ligand to CD47 expressed on the cell surface of a cell; and/or the capability of the test compounds to alter immunoresponsiveness of immune cells. Compounds so identified as capable of influencing CD47 function are valuable for therapeutic and/or diagnostic purposes because they permit treatment and/or detection of diseases associated with CD47 activity.

Candidate agents further may be provided as members of a combinatorial library, which preferably includes synthetic agents prepared according to a plurality of predetermined chemical reactions performed in a plurality of reaction vessels. For example, various starting compounds may be prepared according to one or more of solid-phase synthesis, recorded random mix methodologies, and recorded reaction split techniques that permit a given constituent to traceably undergo a plurality of permutations and/or combinations of reaction conditions. The resulting products comprise a library that can be screened followed by iterative selection and synthesis procedures, such as a combinatorial library of synthetic peptides (see, e.g., International Patent Application Nos. PCT/US91/08694 and PCT/US91/04666) or other compositions that may include small molecules (see, e.g., International Patent Application No. PCT/US94/08542, EP Patent No. 0774464, U.S. Pat. No. 5,798,035, U.S. Pat. No. 5,789,172, U.S. Pat. No. 5,751,629, which are hereby incorporated by reference in their entireties). Those having ordinary skill in the art will appreciate that a diverse assortment of such libraries may be prepared according to established procedures and tested according to the present disclosure. Certain combinatorial libraries of small molecules and combinatorial libraries of peptides may also be obtained commercially.

Examples of methods for the synthesis of molecular libraries can be found in the art, for example in DeWitt et al., Proc. Natl. Acad. Sci. U.S.A. 90:6909 (1993); Erb et al., Proc. Natl. Acad. Sci. USA 91:11422 (1994); Zuckermann et al., J. Med. Chem. 37:2678 (1994); Cho et al., Science 261:1303 (1993); Carrell et al., Angew. Chem. Int. Ed. Engl. 33:2059 (1994); Carell et al., Angew. Chem. Int. Ed. Engl. 33:2061 (1994); and in Gallop et al., J. Med. Chem. 37:1233 (1994). Libraries of compounds may be presented in solution (e.g., Houghten, Biotechniques 13:412-21 (1992)); or on beads (Lam, Nature 354:82-84 (1991)); chips (Fodor, Nature 364:555-56 (1993)); bacteria (Ladner, U.S. Pat. No. 5,223,409); spores (Ladner, supra); plasmids (Cull et al., Proc. Natl. Acad. Sci. USA 89:1865-69 (1992)); or on phage (Scott and Smith, Science 249:386-390 (1990); Devlin, Science 249:404-406 (1990); Cwirla et al., Proc. Natl. Acad. Sci. USA 87:6378-82 (1990); Felici, J. Mol. Biol. 222:301-10 (1991); Ladner, supra).

Peptide-Immunoglobulin Constant Region Fusion Polypeptides

In one embodiment, a bioactive agent that is used for altering the immunoresponsiveness of an immune cell and that may be used for treating an immunological disease or disorder is a peptide-immunoglobulin (Ig) constant region fusion polypeptide, which includes a peptide-IgFc fusion polypeptide. The peptide may be any naturally occurring or recombinantly prepared molecule. A peptide-Ig constant region fusion polypeptide, such as a peptide-IgFc fusion polypeptide (also referred to in the art as a peptibody (see, e.g., U.S. Pat. No. 6,660,843)), comprises a biologically active peptide or polypeptide capable of altering the activity of a protein of interest, such as CD47 expressed by an immune cell or a CD47 ligand. The peptide may be fused in-frame with a portion, at least one constant region domain (e.g., CH1, CH2, CH3, and/or CH4), or the Fc polypeptide (CH2-CH3) of an immunoglobulin. An Fc polypeptide, which includes a mutein Fc polypeptide is described herein in detail, is also referred to herein as the Fc portion or the Fc region.

In one embodiment, the peptide portion of the fusion polypeptide is capable of interacting with or binding to CD47, and effecting the same biological activity as a viral CD47-like polypeptide when it binds to CD47, and/or effecting the same biological activity as a CD47 ligand, thus suppressing (inhibiting, preventing, decreasing, or abrogating) the immunoresponsiveness of an immune cell. Methods are provided herein for identifying a peptide that is capable of altering (e.g., suppressing) immunoresponsiveness of an immune cell (that is, a peptide that acts as viral CD47-like polypeptide mimic). For example, such a peptide may be identified by determining its capability to inhibit or block binding of a CD47 ligand to a cell that expresses CD47. Alternatively, a candidate peptide may be permitted to contact or interact with an immune cell that expresses CD47, and the capability of the candidate peptide to suppress or enhance immunoresponsiveness of the immune cell can be measured according to methods described herein and practiced in the art. Candidate peptides may be provided as members of a combinatorial library, which includes synthetic peptides prepared according to a plurality of predetermined chemical reactions performed in a plurality of reaction vessels. For example, various starting peptides may be prepared according to standard peptide synthesis techniques with which a skilled artisan will be familiar.

Peptides that alter the immunoresponsiveness of an immune cell may be identified and isolated from combinatorial libraries (see, e.g., International Patent Application Nos. PCT/US91/08694 and PCT/US91/04666) and from phage display peptide libraries (see, e.g., Scott et al., Science 249:386 (1990); Devlin et al., Science 249:404 (1990); Cwirla et al., Science 276: 1696-99 (1997); U.S. Pat. No. 5,223,409; U.S. Pat. No. 5,733,731; U.S. Pat. No. 5,498,530; U.S. Pat. No. 5,432,018; U.S. Pat. No. 5,338,665; 1994; U.S. Pat. No. 5,922,545; International Application Publication Nos. WO 96/40987 and WO 98/15833). In phage display peptide libraries, random peptide sequences are fused to a phage coat protein such that the peptides are displayed on the external surface of a filamentous phage particle. Typically, the displayed peptides are contacted with a ligand or binding molecule of interest to permit interaction between the peptide and the ligand or binding molecule, unbound phage are removed, and the bound phage are eluted and subsequently enriched by successive rounds of affinity purification and repropagation. The peptides with the greatest affinity for the ligand or binding molecule or target molecule of interest (e.g., CD47) may be sequenced to identify key residues, which may identify peptides within one or more structurally related families of peptides. Comparison of sequences of peptides may also indicate which residues in such peptides may be safely substituted or deleted by mutagenesis. These peptides may then be incorporated into additional peptide libraries that can be screened and peptides with optimized affinity can be identified.

Additional methods for identifying peptides that may alter the immunoresponsiveness of an immune cell and thus be useful for treating and/or preventing an immunological disease or disorder include, but are not limited to, (1) structural analysis of protein-protein interaction such as analyzing the crystal structure of the CD47 target (particularly the CD47 extracellular domain) (see, e.g., Jia, Biochem. Cell Biol. 75:17-26 (1997)) to identity and to determine the orientation of critical residues of CD47, which will be useful for designing a peptide (see, e.g., Takasaki et al., Nature Biotech. 15: 1266-70 (1997)); (2) a peptide library comprising peptides fused to a peptidoglycan-associated lipoprotein and displayed on the outer surface of bacteria such as E. coli; (3) generating a library of peptides by disrupting translation of polypeptides to generate RNA-associated peptides; and (4) generating peptides by digesting polypeptides with one or more proteases. (See also, e.g., U.S. Pat. Nos. 6,660,843; 5,773,569; 5,869,451; 5,932,946; 5,608,035; 5,786,331; 5,880,096). A peptide may comprise any number of amino acids between 3 and 75 amino acids, 3 and 60 amino acids, 3 and 50 amino acids, 3 and 40 amino acids, 3 and 30 amino acids, 3 and 20 amino acids, or 3 and 10 amino acids. A peptide that has the capability of alter the immunoresponsiveness of an immune cell (e.g., in certain embodiments, to suppress the immunoresponsiveness of the immune cell and in certain other embodiments, to enhance immunoresponsiveness of the immune cell) may also be further derivatized to add or insert amino acids that are useful for constructing a peptide-Ig constant region fusion protein (such as amino acids that are linking sequences or that are spacer sequences).

A peptide that may be used to construct a peptide-Ig constant region fusion polypeptide (including a peptide-IgFc fusion polypeptide) may be derived from a CD47 ligand (e.g., SIRP-α, SIRP-beta-2, thrombospondin-1, αᵥβ₃ integrin, and α₂β₁). Peptides may be randomly generated by proteolytic digestion of a CD47 ligand using any one or more of various proteases, isolated, and then analyzed for their capability to alter the immunoresponsiveness of an immune cell. CD47 ligand peptides may also be generated using recombinant methods described herein and practiced in the art. Randomly generated peptides may also be used to prepare peptide combinatorial libraries or phage libraries as described herein and in the art. Alternatively, the amino acid sequences of portions of a CD47 ligand that interact with CD47 may be determined by computer modeling of CD47, or of a portion of CD47, for example, the extracellular portion thereof, and/or x-ray crystallography (which may include preparation and analysis of crystals of the CD47 extracellular domain only or of the CD47 extracellular domain complexed with a CD47 ligand).

The IgFc portion of a peptide-IgFc fusion polypeptide may be any of the Fc polypeptides or mutein Fc polypeptides that are described in detail herein for fusing to a CD47 extracellular domain. As described in detail above, an Fc polypeptide of an immunoglobulin comprises the heavy chain CH2 domain and CH3 domain and a portion of or the entire hinge region that is located between CH1 and CH2. Fc regions are monomeric polypeptides that may be linked into dimeric or multimeric forms by covalent (e.g., particularly disulfide bonds) and non-covalent association. The number of intermolecular disulfide bonds between monomeric subunits of Fc polypeptides varies depending on the immunoglobulin class (e.g., IgG, IgA, IgE) or subclass (e.g., human IgG1, IgG2, IgG3, IgG4, IgA1, IgA2). An Fc polypeptide, and any one or more constant region domains, and fusion proteins comprising at least one immunoglobulin (Ig) constant region domain can be readily prepared according to recombinant molecular biology techniques with which a skilled artisan is quite familiar.

The Fc polypeptide is preferably prepared using the nucleotide and the encoded amino acid sequences derived from the animal species for whose use the peptide-IgFc fusion polypeptide is intended. In one embodiment, the Fc polypeptide is of human origin and may be from any of the immunoglobulin classes, such as human IgG1 and IgG2.

An Fc polypeptide as described herein also includes Fc polypeptide variants (also referred to herein as mutein Fc polypeptides). An Fc polypeptide or mutein Fc polypeptide that may be fused to a peptide as described above includes any one of the mutein Fc polypeptides described above. One such Fc polypeptide variant has one or more cysteine residues (such as one or more cysteine residues in the hinge region) substituted with another amino acid, such as serine, or deleted to reduce the number of disulfide bonds that may form between two Fc polypeptide monomers.

Also as described in detail herein, another example of an Fc polypeptide variant is a variant that has one or more amino acids involved in, or associated with, an effector function substituted or deleted such that the Fc polypeptide has a reduced level of an effector function. For example, amino acids in the Fc region may be substituted to reduce or abrogate binding of a component of the complement cascade (see, e.g., Duncan et al., Nature 332:563-64 (1988); Morgan et al., Immunology 86:319-24 (1995)) or to reduce or abrogate the ability of the Fc polypeptide to bind to an IgG Fc receptor expressed by an immune cell (Wines et al., J. Immunol. 164:5313-18 (2000); Chappel et al., Proc. Natl. Acad. Sci. USA 88:9036 (1991); Canfield et al., J. Exp. Med. 173:1483 (1991); Duncan et al., supra); or to alter antibody-dependent cellular cytotoxicity.

In certain embodiments, a mutein Fc polypeptide that is fused with a peptide comprises a substitution or a deletion of the cysteine residue that is most proximal to the amino terminus of the hinge region of an Fc polypeptide and a deletion of the adjacent aspartic acid residue (toward the C-terminal end of the Fc polypeptide). The mutein Fc polypeptide may further comprise at least one, two, or three or more amino acid substitutions in the CH2 domain of the Fc polypeptide. In a particular embodiment, at least one of the amino acid substitutions in the CH2 domain reduces the capability of the mutein Fc polypeptide to bind to an IgFc receptor. In specific embodiments, the at least one, two, or three amino acids substitutions in the CH2 domain are substitutions of an amino acid(s) located at a position that corresponds to EU numbered position 234, 235, and/or 237. Exemplary mutein Fc polypeptides are described in detail herein and exemplary amino acid sequences of mutein Fc polypeptides include, but are not limited to, the amino acid sequences set forth in SEQ ID NOSs:7, 8 and 23.

Aptamers

Aptamers are DNA or RNA molecules, generally single-stranded, that have been selected from random pools based on their ability to bind other molecules, including nucleic acids, proteins, lipids, etc. Unlike antisense polynucleotides, short interfering RNA (siRNA), or ribozymes that bind to a polynucleotide that comprises a sequence that encodes a polypeptide of interest and that alter transcription or translation, aptamers can target and bind to polypeptides. Aptamers may be selected from random or unmodified oligonucleotide libraries by their ability to bind to specific targets, in this instance, CD47 (see, e.g., U.S. Pat. No. 6,867,289; U.S. Pat. No. 5,567,588). Aptamers have capacity to form a variety of two- and three-dimensional structures and have sufficient chemical versatility available within their monomers to act as ligands (i.e., to form specific binding pairs) with virtually any chemical compound, whether monomeric or polymeric. Molecules of any size or composition can serve as targets. An iterative process of in vitro selection may be used to enrich the library for species with high affinity to the target. This process involves repetitive cycles of incubation of the library with a desired target, separation of free oligonucleotides from those bound to the target, and amplification of the bound oligonucleotide subset, such as by using the polymerase chain reaction (PCR). From the selected sub-population of sequences that have high affinity for the target, a sub-population may be subcloned and particular aptamers examined in further detail to identify aptamers that alter a biological function of the target (see, e.g., U.S. Pat. No. 6,699,843).

Aptamers may comprise any deoxyribonucleotide or ribonucleotide or modifications of these bases, such as deoxythiophosphosphate (or phosphorothioate), which have sulfur in place of oxygen as one of the non-bridging ligands bound to the phosphorus. Monothiophosphates αS have one sulfur atom and are thus chiral around the phosphorus center. Dithiophosphates are substituted at both oxygens and are thus achiral. Phosphorothioate nucleotides are commercially available or can be synthesized by several different methods known in the art.

Expression of a Fusion Polypeptide Comprising CD47 Extracellular Domain, a Fusion Polypeptide Comprising a viral CD47-like Extracellular Domain, and Polypeptide Agents

Any of the fusion polypeptides described herein, including a fusion polypeptide comprising a CD47 extracellular domain, or variant thereof, fused to a mutein Fc polypeptide, a fusion polypeptide comprising a viral CD47-like extracellular domain fused to a mutein Fc polypeptide, and peptide-IgFc fusion polypeptides, may be expressed using vectors and constructs, particularly recombinant expression constructs, that include any polynucleotide encoding such polypeptides. Host cells are genetically engineered with vectors and/or constructs to produce these polypeptides and fusion proteins, or fragments or variants thereof, by recombinant techniques. Each of the polypeptides and fusion polypeptides described herein can be expressed in mammalian cells, yeast, bacteria, insect, or other cells under the control of appropriate promoters. Cell-free translation systems can also be employed to produce such proteins using RNAs derived from DNA constructs. Appropriate cloning and expression vectors for use with prokaryotic and eukaryotic hosts are described, for example, by Sambrook, et al., Molecular Cloning: A Laboratory Manual, Third Edition, Cold Spring Harbor, N.Y., (2001).

Generally, recombinant expression vectors include origins of replication, selectable markers permitting transformation of the host cell, for example, the ampicillin resistance gene of E. coli and S. cerevisiae TRP1 gene, and a promoter derived from a highly expressed gene to direct transcription of a downstream structural sequence. Promoters can be derived from operons encoding glycolytic enzymes such as 3-phosphoglycerate kinase (PGK), α-factor, acid phosphatase, or heat shock proteins, among others. The heterologous structural sequence is assembled in appropriate phase with translation initiation and termination sequences.

Optionally, a heterologous sequence can encode a fusion protein that further includes an amino terminal or carboxy terminal identification peptide or polypeptide that imparts desired characteristics, e.g., that stabilizes the produced polypeptide or that simplifies purification of the expressed recombinant product. Such identification peptides include a polyhistidine tag (his tag) or FLAG® epitope tag (DYKDDDDK, SEQ ID NO:24), beta-galactosidase, alkaline phosphatase, GST, or the XPRESS™ epitope tag (DLYDDDDK, SEQ ID NO:25; Invitrogen Life Technologies, Carlsbad, Calif.) and the like (see, e.g., U.S. Pat. No. 5,011,912; Hopp et al., (Bio/Technology 6:1204 (1988)). The affinity sequence may be supplied by a vector, such as, for example, a hexa-histidine tag that is provided in pBAD/His (Invitrogen). Alternatively, the affinity sequence may be added either synthetically or engineered into the primers used to recombinantly generate the nucleic acid coding sequence (e.g., using the polymerase chain reaction).

Host cells containing described recombinant expression constructs may be genetically engineered (transduced, transformed, or transfected) with the vectors and/or expression constructs (for example, a cloning vector, a shuttle vector, or an expression construct). The vector or construct may be in the form of a plasmid, a viral particle, a phage, etc. The engineered host cells can be cultured in conventional nutrient media modified as appropriate for activating promoters, selecting transformants, or amplifying particular genes or encoding-nucleotide sequences. Selection and maintenance of culture conditions for particular host cells, such as temperature, pH and the like, will be readily apparent to the ordinarily skilled artisan. Preferably the host cell can be adapted to sustained propagation in culture to yield a cell line according to art-established methodologies. In certain embodiments, the cell line is an immortal cell line, which refers to a cell line that can be repeatedly (at least ten times while remaining viable) passaged in culture following log-phase growth. In other embodiments the host cell used to generate a cell line is a cell that is capable of unregulated growth, such as a cancer cell, or a transformed cell, or a malignant cell.

Useful bacterial expression constructs are constructed by inserting into an expression vector a structural DNA sequence encoding a desired protein together with suitable translation initiation and termination signals in operable reading phase with a functional promoter. The construct may comprise one or more phenotypic selectable markers and an origin of replication to ensure maintenance of the vector construct and, if desirable, to provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice. Any other plasmid or vector may be used as long as they are replicable and viable in the host. Thus, for example, the nucleic acids as provided herein may be included in any one of a variety of expression vector constructs as a recombinant expression construct for expressing a polypeptide. Such vectors and constructs include chromosomal, nonchromosomal, and synthetic DNA sequences, e.g., bacterial plasmids; phage DNA; baculovirus; yeast plasmids; vectors derived from combinations of plasmids and phage DNA; viral DNA, such as vaccinia, adenovirus, fowl pox virus, and pseudorabies. However, any other vector may be used for preparation of a recombinant expression construct as long as it is replicable and viable in the host.

The appropriate DNA sequence(s) may be inserted into the vector by a variety of procedures. In general, the DNA sequence is inserted into an appropriate restriction endonuclease site(s) by procedures known in the art. Standard techniques for cloning, DNA isolation, amplification and purification, for enzymatic reactions involving DNA ligase, DNA polymerase, restriction endonucleases and the like, and various separation techniques are those known and commonly employed by those skilled in the art. Numerous standard techniques are described, for example, in Ausubel et al. (Current Protocols in Molecular Biology (Greene Publ. Assoc. Inc. & John Wiley & Sons, Inc., 1993)); Sambrook et al. (Molecular Cloning. A Laboratory Manual, 3rd Ed., (Cold Spring Harbor Laboratory 2001)); Maniatis et al. (Molecular Cloning, (Cold Spring Harbor Laboratory 1982)), and elsewhere.

The DNA sequence encoding a polypeptide in the expression vector is operatively linked to at least one appropriate expression control sequences (e.g., a promoter or a regulated promoter) to direct mRNA synthesis. Representative examples of such expression control sequences include LTR or SV40 promoter, the E. coli lac or trp, the phage lambda P_{L} promoter, and other promoters known to control expression of genes in prokaryotic or eukaryotic cells or their viruses. Promoter regions can be selected from any desired gene using CAT (chloramphenicol transferase) vectors or other vectors with selectable markers. Particular bacterial promoters include lac, lacZ, T3, T5, T7, gpt, lambda P_{R}, P_{L}, and trp.

Eukaryotic promoters include CMV immediate early, HSV thymidine kinase, early and late SV40, LTRs from retroviruses, and mouse metallothionein-I. Selection of the appropriate vector and promoter and preparation of certain recombinant expression constructs comprising at least one promoter or regulated promoter operatively linked to a nucleic acid described herein is well within the level of ordinary skill in the art.

Design and selection of inducible, regulated promoters and/or tightly regulated promoters are known in the art and will depend on the particular host cell and expression system. The pBAD Expression System (Invitrogen Life Technologies, Carlsbad, Calif.) is an example of a tightly regulated expression system that uses the E. coli arabinose operon (P_{BAD} or P_{ARA}) (see Guzman et al., J. Bacteriology 177:4121-30 (1995); Smith et al., J. Biol. Chem. 253:6931-33 (1978); Hirsh et al., Cell 11:545-50 (1977)), which controls the arabinose metabolic pathway. A variety of vectors employing this system are commercially available. Other examples of tightly regulated promoter-driven expression systems include PET Expression Systems (see U.S. Pat. No. 4,952,496) available from Stratagene (La Jolla, Calif.) or tet-regulated expression systems (Gossen et al., Proc. Natl. Acad. Sci. USA 89:5547-51 (1992); Gossen et al., Science 268:1766-69 (1995)). The pLP-TRE2 Acceptor Vector (BD Biosciences Clontech, Palo Alto, Calif.) is designed for use with CLONTECH's Creator™ Cloning Kits to rapidly generate a tetracycline-regulated expression construct for tightly controlled, inducible expression of a gene of interest using the site-specific Cre-lox recombination system (see, e.g., Sauer, Methods 14:381-92 (1998); Furth, J. Mamm. Gland Biol. Neoplas. 2:373 (1997)), which may also be employed for host cell immortalization (see, e.g., Cascio, Artif. Organs 25:529 (2001)).

The vector may be a viral vector such as a retroviral vector. For example, retroviruses from which the retroviral plasmid vectors may be derived include, but are not limited to, Moloney Murine Leukemia Virus, spleen necrosis virus, Rous Sarcoma Virus, Harvey Sarcoma virus, avian leukosis virus, gibbon ape leukemia virus, human immunodeficiency virus, adenovirus, Myeloproliferative Sarcoma Virus, and mammary tumor virus. A viral vector also includes one or more promoters. Suitable promoters that may be employed include, but are not limited to, the retroviral LTR; the SV40 promoter; and the human cytomegalovirus (CMV) promoter described in Miller et al., Biotechniques 7:980-990 (1989), or any other promoter (e.g., eukaryotic cellular promoters including, for example, the histone, pol III, and β-actin promoters). Other viral promoters that may be employed include, but are not limited to, adenovirus promoters, thymidine kinase (TK) promoters, and B 19 parvovirus promoters.

The retroviral plasmid vector is employed to transduce packaging cell lines (e.g., PE501, PA317, ψ-2, ψ-AM, PA12, T19-14X, VT-19-17-H2, ψCRE, ψCRIP, GP+E-86, GP+envAm12, DAN; see also, e.g., Miller, Human Gene Therapy, 1:5-14 (1990)) to form producer cell lines. The vector may transduce the packaging cells through any means known in the art, such as, for example, electroporation, the use of liposomes, and calcium phosphate precipitation. The producer cell line generates infectious retroviral vector particles that include the nucleic acid sequence(s) encoding the polypeptides or fusion proteins described herein. Such retroviral vector particles then may be employed, to transduce eukaryotic cells, either in vitro or in vivo. Eukaryotic cells that may be transduced include, for example, embryonic stem cells, embryonic carcinoma cells, hematopoietic stem cells, hepatocytes, fibroblasts, myoblasts, keratinocytes, endothelial cells, bronchial epithelial cells, and other culture-adapted cell lines.

As another example, host cells transduced by a recombinant viral construct directing the expression of polypeptides or fusion proteins may produce viral particles containing expressed polypeptides or fusion proteins that are derived from portions of a host cell membrane incorporated by the viral particles during viral budding. The polypeptide-encoding nucleic acid sequences may be cloned into a baculovirus shuttle vector, which is then recombined with a baculovirus to generate a recombinant baculovirus expression construct that is used to infect, for example, Sf9 host cells (see, e.g., Baculovirus Expression Protocols, Methods in Molecular Biology Vol. 39, Richardson, Ed. (Human Press 1995); Piwnica-Worms, “Expression of Proteins in Insect Cells Using Baculoviral Vectors,” Section II, Chapter 16 in Short Protocols in Molecular Biology, 2^{nd} Ed., Ausubel et al., eds., (John Wiley & Sons 1992), pages 16-32 to 16-48).

### Treatment of Immunological Disorders and Disease

In another embodiment, methods are provided for treating and/or preventing immunological diseases and disorders, particularly an inflammatory disease or disorder, an autoimmune disease or disorder, cardiovascular disease or disorder, a metabolic disease or disorder, or a proliferative disease or disorder as described herein. A subject in need of such treatment may be a human or may be a non-human primate or other animal (i.e., veterinary use) who has developed symptoms of an immunological disease or who is at risk for developing an immunological disease. Examples of non-human primates and other animals include but are not limited to farm animals, pets, and zoo animals (e.g., horses, cows, buffalo, llamas, goats, rabbits, cats, dogs, chimpanzees, orangutans, gorillas, monkeys, elephants, bears, large cats, etc.).

As described herein, a method is provided for altering (e.g., suppressing or enhancing) an immune response in a subject (host or patient) who has or who is at risk for developing an immunological disease or disorder, by administering a composition that comprises a pharmaceutically acceptable carrier (also referred to herein as a pharmaceutically or physiologically suitable carrier or excipient) and any one of the fusion polypeptides described herein that comprise a CD47 extracellular domain, or variant thereof, fused to a Fc polypeptide, or variant thereof (which also includes fusion polypeptide dimers). In certain embodiments, the composition suppresses the immune response by suppressing (i.e., decreasing, reducing, inhibiting, abrogating) immunoresponsiveness of an immune cell. In other embodiments, CD47-Fc polypeptide fusion proteins described herein suppress immunoresponsiveness of an immune cell and inhibit (i.e., decrease, reduce, suppress, abrogate) the production of at least one cytokine and/or at least one chemokine by an immune cell. In other embodiments, the fusion proteins block or inhibit interaction between an immune complex and an immune cell, such as a dendritic cell, and thus inhibits the production of cytokines and/or chemokines by the immune cell, for example, a dendritic cell. The fusion polypeptides described herein may also alter cell migration; inhibit (i.e., decrease, block, reduce, abrogate) production of at least one cytokine, including but not limited to, TNF-α, IL-12, IL-23 IFN-γ, GM-CSF, and IL-6; inhibit maturation of a dendritic cell; impair (i.e., inhibit, prevent, slow, or in some manner deleteriously affect) development of a naïve T cell into a Th1 effector cell; inhibit (i.e., decrease, block, reduce, abrogate) immune complex-induced production of a cytokine (e.g., TNF-α, IL-12, IL-23 IFN-γ, GM-CSF, and IL-6) by an immune cell (e.g., a dendritic cell); inhibit (i.e., decrease, block, reduce, abrogate) Fc-mediated production of a cytokine (e.g., TNF-α, IL-12, IL-23 IFN-γ, GM-CSF, and IL-6) by an immune cell (e.g., a dendritic cell); suppress (i.e., inhibit, decrease, block, reduce, abrogate) cytokine and/or chemokine secretion by an immune cell, including but not limited to a dendritic cell; inhibit activation of an immune cell wherein the immune cell expresses SIRP a on the cell surface; suppress a proinflammatory response in a biologically or clinically significant manner.

In other certain embodiments, the composition comprises an antibody, or antigen-binding fragment thereof, that specifically binds to CD47 and a pharmaceutically acceptable (i.e., suitable) carrier. In another certain embodiment, a composition is provided that comprises an agent that binds to CD47, such as a small molecule, an aptamer, or a peptide Fc fusion polypeptide and a pharmaceutically acceptable (i.e., suitable) carrier.

Also provided is a method for altering (e.g., suppressing or enhancing) an immune response in a subject (host or patient) who has or who is at risk for developing an immunological disease or disorder, by administering any one of the aforementioned compositions. In one embodiment, a method for altering an immune response in a subject comprises administering a composition that comprises a pharmaceutically suitable carrier and any one of the fusion polypeptides described herein that comprise a CD47 extracellular domain, or variant thereof, fused to an Fc polypeptide or variant thereof; a composition that comprises a pharmaceutically suitable carrier and at least one antibody, or antigen-binding fragment thereof, that specifically binds to CD47, as described in detail herein. In another embodiment, a method of altering an immune response in a subject comprises administering a composition comprising an agent that binds to CD47 as described in detail herein, such as a small molecule, an aptamer, or a peptide Fc fusion polypeptide and a pharmaceutically suitable carrier. In a particular embodiment, such a method suppresses an immune response in a subject. In another particular embodiment, such a method enhances an immune response in a subject.

In another embodiment, a method for treating an immunological disease or disorder is provided wherein the method comprises administering to a subject in need thereof a pharmaceutically suitable carrier and an agent that alters a biological activity of at CD47. An agent as described herein (including an antibody, or antigen-binding fragment thereof, for example an antibody that specifically binds to a CD47 ligand, such as SIRPalpha); a small molecule; an aptamer; a peptide-IgFc fusion polypeptide or peptide Ig constant region domain fusion polypeptide; and a fusion polypeptide comprising a CD47 extracellular domain, all of which are described in detail herein) that is useful for treating an immunological disease or disorder is capable of altering (increasing or decreasing in a statistically significant or biological significant manner) at least one biological activity (function) of CD47. Such an agent used for treating an immunological disease or disorder may therefore affect or alter any one or more of the biological activities or functions of CD47, including at least one of the following: altering immunoresponsiveness of an immune cell; altering cell migration; inhibiting production of at least one cytokine selected from TNF-α, IL-12, IL-23 IFN-γ, GM-CSF, and IL-6; inhibiting maturation of a dendritic cell; impairing development of a naïve T cell into a Th1 effector cell; inhibiting immune complex-induced production of a cytokine (e.g., TNF-α, IL-12, IL-23, IFN-γ, GM-CSF, and IL-6) by an immune cell (e.g., a dendritic cell); suppressing cytokine secretion by a dendritic cell; and suppressing a proinflammatory response.

Methods are also provided for treating an immunological disease or disorder in a subject in need thereof, wherein the composition administered to the subject comprises a pharmaceutically suitable carrier and an agent, which agent has the capability to specifically bind to CD47 and to impair (i.e., inhibit, prevent, reduce) binding of a viral CD47-like polypeptide to at least one CD47 ligand (e.g., SIRP-α, SIRP-beta-2, thrombospondin-1, αᵥβ₃ integrin, and α₂β₁ integrin). The agent also includes an antibody, or antigen-binding fragment thereof that specifically binds to a CD47 ligand (e.g., different antibodies that each specifically bind to the respective cognate ligand, such as SIRP-α, SIRP-beta-2, thrombospondin-1, αᵥβ₃ integrin, and α₂β₁ integrin) The capability of the agent to bind to CD47 and to impair binding of the vCD47 to at least one CD47 ligand may be determined by employing assays and techniques described herein using an isolated CD47 polypeptide (or fragment thereof, such as the extracellular domain) or using a cell that expresses CD47 on its surface. The vCD47 polypeptide may be any one of the poxvirus CD47-like polypeptides described herein, for example, variola minor CD47-like polypeptide, which has the amino acid sequence set forth in SEQ ID NO:3. An agent that specifically binds to CD47 and that impairs binding of a viral CD47-like polypeptide to a CD47 ligand may also be used for treating a disease or disorder that is associated with alteration of at least one of cell migration, cell proliferation, and cell differentiation.

The fusion polypeptides, agents, and methods described herein may be used for treating (i.e., curing, preventing, ameliorating the symptoms of, or slowing, inhibiting, or stopping the progression of) an immunological disease or disorder. Such diseases and disorders that are autoimmune or inflammatory disorders include but are not limited to multiple sclerosis, rheumatoid arthritis, an antibody-mediated inflammatory disease or disorder, a spondyloarthropathy, systemic lupus erythematosus, graft versus host disease, sepsis, diabetes, psoriasis, atherosclerosis, Sjogren's syndrome, progressive systemic sclerosis, scieroderma, acute coronary syndrome, ischemic reperfusion, Crohn's Disease, endometriosis, glomerulonephritis, myasthenia gravis, idiopathic pulmonary fibrosis, asthma, acute respiratory distress syndrome (ARDS), vasculitis, or inflammatory autoimmune myositis. A spondyloarthropathy includes, for example, ankylosing spondylitis, reactive arthritis, enteropathic arthritis associated with inflammatory bowel disease, psoriatic arthritis, isolated acute anterior uveitis, undifferentiated spondyloarthropathy, Behcet's syndrome, and juvenile idiopathic arthritis.

An immunological disorder or disease also includes a cardiovascular disease or disorder, a metabolic disease or disorder, or a proliferative disease or disorder. A cardiovascular disease or disorder that may be treated according to the methods and with the fusion polypeptides and agents described herein includes, for example, atherosclerosis, endocarditis, hypertension, or peripheral ischemic disease. Metabolic diseases that also are immunological disorders or diseases include diabetes, Crohn's Disease, and inflammatory bowel disease. An exemplary proliferative disease is cancer. A cancer or malignancy includes, but is not limited to, a leukemia (e.g., B-cell chronic lymphocytic leukemia), lymphoma, breast cancer, renal cancer, and ovarian cancer.

As used herein, a patient (or subject) may be any mammal, including a human, that may have or be afflicted with an immunological disease or disorder, or that may be free of detectable disease. Accordingly, the treatment may be administered to a subject who has an existing disease, or the treatment may be prophylactic, administered to a subject who is at risk for developing the disease or condition.

A composition may be a pharmaceutical composition that is a sterile aqueous or non-aqueous solution, suspension or emulsion, which additionally comprises a physiologically acceptable or suitable carrier. A pharmaceutically acceptable or suitable carrier may include (or refer to) an excipient (i.e., a non-toxic material that does not interfere with the activity of the active ingredient) and/or a diluent. Such compositions may be in the form of a solid, liquid, or gas (aerosol). Alternatively, compositions described herein may be formulated as a lyophilizate, or compounds may be encapsulated within liposomes using technology known in the art. Pharmaceutical compositions may also contain other components, which may be biologically active or inactive. Such components include, but are not limited to, buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, stabilizers, dyes, flavoring agents, and suspending agents and/or preservatives.

Any suitable excipient or carrier known to those of ordinary skill in the art for use in pharmaceutical compositions may be employed in the compositions described herein. Excipients for therapeutic use are well known, and are described, for example, in Remingtons Pharmaceutical Sciences, Mack Publishing Co. (A. R. Gennaro ed. 1985). In general, the type of excipient is selected based on the mode of administration. Pharmaceutical compositions may be formulated for any appropriate manner of administration, including, for example, topical, oral, nasal, intrathecal, rectal, vaginal, intraocular, subconjunctival, sublingual or parenteral administration, including subcutaneous, intravenous, intramuscular, intrasternal, intracavernous, intrameatal or intraurethral injection or infusion. For parenteral administration, the carrier preferably comprises water, saline, alcohol, a fat, a wax or a buffer. For oral administration, any of the above excipients or a solid excipient or carrier, such as mannitol, lactose, starch, magnesium stearate, sodium saccharine, talcum, cellulose, kaolin, glycerin, starch dextrins, sodium alginate, carboxymethylcellulose, ethyl cellulose, glucose, sucrose and/or magnesium carbonate, may be employed.

A pharmaceutical composition (e.g., for oral administration or delivery by injection) may be in the form of a liquid. A liquid pharmaceutical composition may include, for example, one or more of the following: a sterile diluent such as water for injection, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral preparation can be enclosed in ampoules, disposable syringes or multiple dose vials made of glass or plastic. The use of physiological saline is preferred, and an injectable pharmaceutical composition is preferably sterile.

The fusion polypeptides and agents described herein, including antibodies and antigen-binding fragments thereof that specifically bind to CD47, small molecules, aptamers, and peptide-fusion proteins, may be formulated for sustained or slow release. Such compositions may generally be prepared using well known technology and administered by, for example, oral, rectal or subcutaneous implantation, or by implantation at the desired target site. Sustained-release formulations may contain an agent dispersed in a carrier matrix and/or contained within a reservoir surrounded by a rate controlling membrane. Excipients for use within such formulations are biocompatible, and may also be biodegradable; preferably the formulation provides a relatively constant level of active component release. The amount of active compound contained within a sustained release formulation depends upon the site of implantation, the rate and expected duration of release, and the nature of the condition to be treated or prevented.

The dose of the composition for treating an immunological disease or disorder may be determined according to parameters understood by a person skilled in the medical art. Accordingly, the appropriate dose may depend upon the patient's (e.g., human) condition, that is, stage of the disease, general health status, as well as age, gender, and weight, and other factors familiar to a person skilled in the medical art.

Pharmaceutical compositions may be administered in a manner appropriate to the disease to be treated (or prevented) as determined by persons skilled in the medical art. An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity). For prophylactic use, a dose should be sufficient to prevent, delay the onset of, or diminish the severity of a disease associated with an immunological disease or disorder.

Optimal doses may generally be determined using experimental models and/or clinical trials. The optimal dose may depend upon the body mass, weight, or blood volume of the patient. In general, the amount of polypeptide, such as a fusion polypeptide as described herein or an antibody or antigen-binding fragment thereof as described herein, present in a dose, or produced in situ by DNA present in a dose, ranges from about 0.01 μg to about 1000 μg per kg of host. The use of the minimum dosage that is sufficient to provide effective therapy is usually preferred. Patients may generally be monitored for therapeutic or prophylactic effectiveness using assays suitable for the condition being treated or prevented, which assays will be familiar to those having ordinary skill in the art. When administered in a liquid form, suitable dose sizes will vary with the size of the patient, but will typically range from about 1 ml to about 500 ml (comprising from about 0.01 μg to about 1000 μg per kg) for a 10-60 kg subject.

For pharmaceutical compositions comprising an agent that is a nucleic acid molecule including an aptamer, the nucleic acid molecule may be present within any of a variety of delivery systems known to those of ordinary skill in the art, including nucleic acid, and bacterial, viral and mammalian expression systems such as, for example, recombinant expression constructs as provided herein. Techniques for incorporating DNA into such expression systems are well known to those of ordinary skill in the art. The DNA may also be “naked,” as described, for example, in Ulmer et al., Science 259:1745-49, 1993 and reviewed by Cohen, Science 259:1691-1692, 1993. The uptake of naked DNA may be increased by coating the DNA onto biodegradable beads, which are efficiently transported into the cells.

Nucleic acid molecules may be delivered into a cell according to any one of several methods described in the art (see, e.g., Akhtar et al., Trends Cell Bio. 2:139 (1992); Delivery Strategies for Antisense Oligonucleotide Therapeutics, ed. Akhtar, 1995, Maurer et al., Mol. Membr. Biol. 16:129-40 (1999); Hofland and Huang, Handb. Exp. Pharmacol. 137:165-92 (1999); Lee et al., ACS Symp. Ser. 752:184-92 (2000); U.S. Pat. No. 6,395,713; International Patent Application Publication No. WO 94/02595); Selbo et al., Int. J. Cancer 87:853-59 (2000); Selbo et al., Tumour Biol. 23:103-12 (2002); U.S. Patent Application Publication Nos. 2001/0007666, and 2003/077829). Such delivery methods known to persons having skill in the art, include, but are not restricted to, encapsulation in liposomes, by iontophoresis, or by incorporation into other vehicles, such as biodegradable polymers; hydrogels; cyclodextrins (see, e.g., Gonzalez et al., Bioconjug. Chem. 10: 1068-74 (1999); Wang et al., International Application Publication Nos. WO 03/47518 and WO 03/46185); poly(lactic-co-glycolic)acid (PLGA) and PLCA microspheres (also useful for delivery of peptides and polypeptides and other substances) (see, e.g., U.S. Pat. No. 6,447,796; U.S. Patent Application Publication No. 2002/130430); biodegradable nanocapsules; and bioadhesive microspheres, or by proteinaceous vectors (International Application Publication No. WO 00/53722). In another embodiment, the nucleic acid molecules for use in altering (suppressing or enhancing) an immune response in an immune cell and for treating an immunological disease or disorder can also be formulated or complexed with polyethyleneimine and derivatives thereof, such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives (see also, e.g., U.S. Patent Application Publication No. 2003/0077829).

Also provided herein are methods of manufacture for producing an agent that suppresses immunoresponsiveness of an immune cell. The methods comprise identifying an agent that alters immunoresponsiveness of an immune cell. Identifying an agent may be accomplished by any one of the methods described herein, which includes contacting (i) a candidate agent; (ii) a viral CD47-like polypeptide (which are described in detail herein); and (iii) a CD47 ligand (e.g., SIRP-α, SIRP-beta-2, thrombospondin-1, αᵥβ₃ integrin, and α₂β₁ integrin), under conditions and for a time sufficient to permit interaction between the CD47 ligand and the viral CD47-like polypeptide. The method further comprises determining a level of binding of the viral CD47-like polypeptide to the CD47 ligand in the presence of the candidate agent and comparing a level of binding of the viral CD47-like polypeptide to the CD47 ligand in the absence of the candidate agent. A decrease in the level of binding of the viral CD47-like polypeptide to the CD47 ligand in the presence of the candidate agent indicates that the candidate agent inhibits binding of the viral CD47-like polypeptide to the CD47 ligand. The candidate agent is then contacted with (i.e., mixed, combined, or in some manner permitted to interact with) an immune cell that expresses CD47, and a CD47 ligand under conditions and for a time sufficient to permit interaction between a CD47 ligand and CD47. The level of binding of the CD47 ligand to the immune cell in the presence of the candidate agent is determined and compared with a level of binding of the CD47 ligand to the immune cell in the absence of the candidate agent. A decrease in the level of binding of the CD47 ligand to the immune cell in the presence of the candidate agent indicates that the candidate agent alters immunoresponsiveness of the immune cell. Then the agent is produced according to small scale or large scale manufacturing practices known in the art that are useful and practical for producing the particular agent.

The agent may be any agent described herein, such as, for example, a fusion polypeptide comprising a CD47 extracellular domain or variant thereof, an antibody, or antigen-binding fragment thereof that specifically binds to CD47; a small molecule; an aptamer; and a peptide-IgFc fusion polypeptide, or an antibody or antigen binding fragment thereof that binds specifically to a CD47 ligand. In a particular embodiment, the agent is a fusion polypeptide comprising a CD47 extracellular domain or variant thereof or an antibody, or antigen-binding fragment thereof, which may be produced according to methods described herein and that are adapted for large-scale manufacture. For example, production methods include batch cell culture, which is monitored and controlled to maintain appropriate culture conditions. Purification of the fusion polypeptide or antibody, or antigen-binding fragment thereof, may be performed according to methods described herein and known in the art and that comport with laws and guidelines of domestic and foreign regulatory agencies.

### EXAMPLES

### Example 1

### Inhibition of Staphylococcus Aureus Cowan Strain (Sac)-Induced Cytokine Production in human dendritic cells by a human CD47-Fc polypeptide

As described herein, a cognate ligand of CD47 is the signal-regulator protein alpha (Sirp-α) (see also, e.g., Latour et al., J. Immunol. 167:2547-54 (2001)). The capability of a huCD47 extracellular domain-Fc polypeptide construct to inhibit Staphylococcus aureus cell (SAC)-induced cytokine production in human monocyte derived dendritic cells is described in this example.

The human CD47 extracellular domain fused to a human IgG Fc polypeptide was prepared using molecular biology methods and techniques and protein expression methods and techniques with which persons skilled in the art are familiar. The amino acid sequence of the human CD47 extracellular domain Fc polypeptide used in these examples is provided in SEQ ID NO:2. The polypeptide sequence of the CD47 extracellular domain is provided in SEQ ID NO:11 (with the signal peptide) and in SEQ ID NO:1 (without the signal peptide). The amino acid sequence of the human IgG Fc polypeptide used in these examples is provided in SEQ ID NO:23.

Human monocyte-derived dendritic cells were prepared as described (Probst et al., Eur. J. Immunol. 27:2634-42 (1997)). Peripheral blood mononuclear cells (PBMC) were prepared from heparinized blood of healthy donors by gradient centrifugation in Histopaque-1077 (Sigma-Aldrich, St. Louis, Mo.). For this experiments, PBMCs were obtained from three different donors. Briefly, monocytes were generated by an adherent step by culturing 15×10⁶ PBMC in RPMI (Lonza, Walkersville, Md.) with 2% human serum (HuS) per well of a 6 well plate. After 2 h, adherent cells were washed twice with PBS and then cultured with dendritic cell differentiation medium (Exvivo 15, Lonza), 10 ng/ml human granulocyte-macrophage colony stimulating Factor (GMCSF) (PeproTech®, Rocky Hill, N.J.), 10 ng/ml human IL-4 (PeproTech®, Rocky Hill, N.J.). After one day, non-adherent cells were gently removed and dendritic cells were generated from the remaining cells by culture for an additional 7 days in dendritic cell differentiation medium.

The eight day-old human monocyte-derived dendritic cells (2×10⁴ cells per well of a 96 well plate) from each of the three donors were treated in the presence of IFN-γ (1000 U/ml) for 1 hour with varying concentrations (five-fold dilutions between 0.006 and 20 μg/ml) of either the human CD47-human Fc polypeptide or a control molecule, in this instance, human IgG. The cells were then stimulated overnight with 0.01% SAC (Pansorbin® Cells, Calbiochem, San Diego Calif.). Supernatants from the stimulated cell cultures were collected 18 h after stimulation with SAC and stored immediately at −20° C.

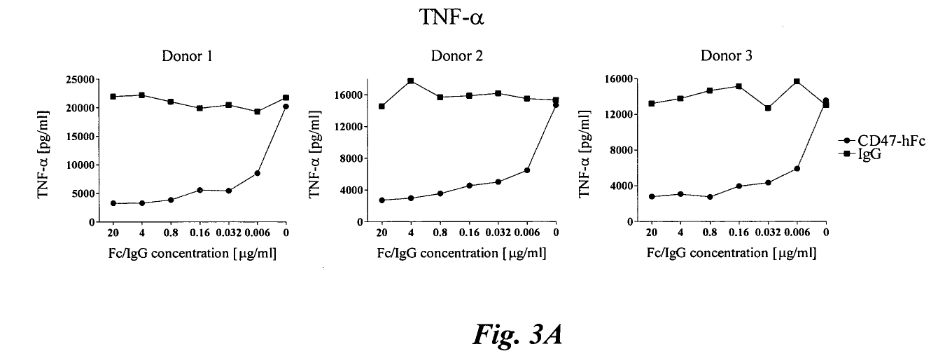

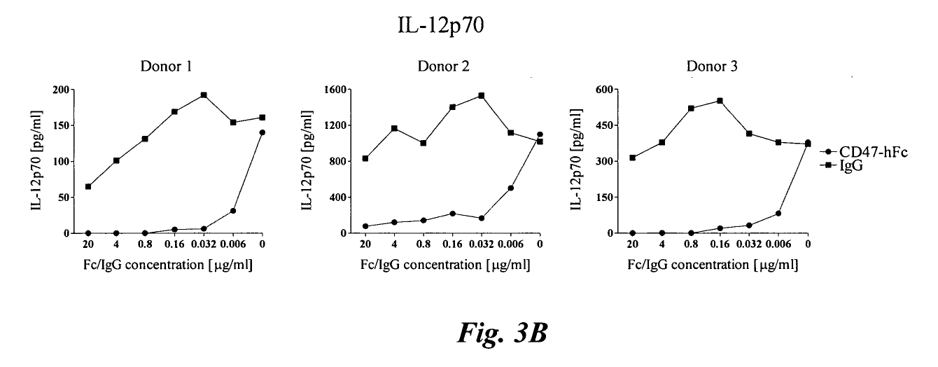

The presence of TNF-α, IL-12p70, IL-6 and MIP-1 alpha in each supernatant was determined by ELISA, using commercially prepared kits according to protocols provided by the manufacturer (R&D Systems, Minneapolis, Minn.). The data are presented in FIG. 3. The hCD47-Fc construct reduced the SAC-induced TNF-α and IL-12p70 production in a dose dependent manner with an IC₅₀ below 6 ng/ml. Human IgG control did not affect SAC-induced TNF-α production and had limited effects on IL-12 production, thus demonstrating that the CD47 domain of the CD47-Fc fusion polypeptide is inhibiting cytokine secretion of DC in response to SAC.

As shown in Table I, human CD47-Fc reduced the secretion of IL-6 and MIP-1 alpha by human dendritic cells in response to PBS-SAC.

TABLE 1
Inhibition of PBS-SAC mediated cytokine production in
human monocyte derived dendritic cells by hCD47-Fc

| | DC Donor 5 | | | | DC Donor 6 | | | |
|---|---|---|---|---|---|---|---|---|
| hCD47-Fc | TNFα | IL-6 | MIP-1α | IL-12 | TNFα | IL-6 | MIP-1α | IL-12 |
| μg/ml | pg/mL | pg/mL | pg/mL | pg/mL | pg/mL | pg/mL | pg/mL | pg/mL |
| 20.0000 | 873 | 961 | 9148 | 41 | 160 | 175 | 1977 | 6 |
| 4.0000 | 958 | 1047 | 10807 | 31 | 198 | 193 | 2381 | 16 |
| 0.8000 | 1199 | 1181 | 10460 | 52 | 320 | 215 | 3323 | 16 |
| 0.1600 | 1379 | 1273 | 13567 | 66 | 576 | 455 | 4742 | 12 |
| 0.0320 | 1413 | 1176 | 13705 | 99 | 722 | 520 | 5155 | 28 |
| 0.0064 | 2627 | 1725 | 15816 | 291 | 1414 | 867 | 6640 | 56 |
| 0.0013 | 4748 | 2338 | 22079 | 594 | 2669 | 1297 | 10420 | 264 |
| 0.0000 | 5375 | 3030 | 21702 | 912 | 2576 | 1119 | 8325 | 173 |

| hFc-Stub | TNFa | IL-6 | MIP-1α | IL-12 | TNFα | IL-6 | MIP-1α | IL-12 |
|---|---|---|---|---|---|---|---|---|
| μg/ml | pg/mL | pg/mL | pg/mL | pg/mL | pg/mL | pg/mL | pg/mL | pg/mL |
| 20.0000 | 5920 | 3602 | 24910 | 934 | 2914 | 1179 | 8565.35 | 128 |
| 4.0000 | 6791 | 3597 | 22991 | 758 | 3425 | 1127 | 9150.823 | 105 |
| 0.8000 | 7911 | 3532 | 29238 | 916 | 3448 | 1553 | 10647.34 | 225 |
| 0.1600 | 7715 | 3332 | 32091 | 829 | 4390 | 1689 | 12610.59 | 296 |
| 0.0320 | 6666 | 3334 | 25806 | 850 | 3861 | 1446 | 10859.59 | 280 |
| 0.0064 | 7707 | 2771 | 19491 | 692 | 3846 | 1661 | 11104.85 | 345 |
| 0.00 | 5371 | 2955 | 19281 | 826 | 2569 | 1291 | 8596.011 | 219 |

Incubation of SAC with human serum (HuS-SAC) increased SAC-induced TNF-α and IL-23 production in human DC. Heat-inactivated human serum (prepared from a normal healthy donor pool) (100 μl) was incubated with an equal volume of 12% SAC for one hour, washed with PBS, and then resuspended with PBS to a final concentration of 1.2% SAC. Preparation of PBS-SAC or FBS (fetal bovine serum)-SAC followed the same protocol by adding PBS or FBS, respectively, instead of human serum.

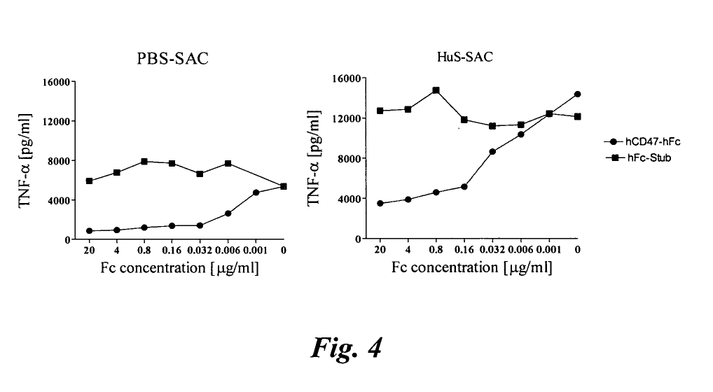

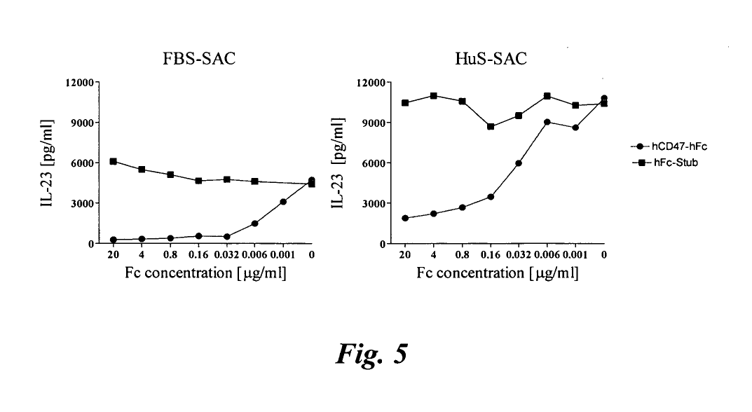

Eight day-old human monocyte-derived DC (2×10⁴ cells/96-well) (prepared as described above) were treated for 1 h in the presence of IFN-γ (1000 U/ml) with varying concentrations (five-fold dilutions between 0.001 and 20 μg/ml) of either the human CD47-human Fc polypeptide or a control molecule, in this instance, a human Fc polypeptide that was not fused to a CD47 extracellular domain (hFc-Stub). Then, DC were stimulated with 0.01% HuS-SAC or with 0.01% PBS-SAC (used in experiments that measured TNF-α) or with 0.01% FBS-SAC (used in experiments that measured IL-23). Supernatants were collected from the stimulated cell cultures after 18 h and stored as described above until the concentrations of TNF-α and IL-23 were determined. The presence of TNF-α was determined as described above. The presence of IL-23 in the supernatants was determined using a commercially prepared kit according to the manufacturer's instructions (eBioscience, San Diego Calif.). The results are presented in FIGS. 4 and 5.

Incubation of SAC with human sera increased SAC-induced TNF-α and IL-23 production in human DC. hCD47-Fc inhibited HuS-SAC induced TNF-α and IL-23 production with an IC₅₀ below 0.16 μg/ml, whereas the Fc-Stub control had no effect on cytokine secretion. Without wishing to be bound by theory, the human IgG present in the human sera may saturate the protein A binding sites on SAC and trigger an Fc-receptor-mediated enhancement of cytokine secretion.

### Example 2

### Inhibition of Immune Complex-Induced Cytokine Production in Human Dendritic Cells by a Human CD47-Fc Polypeptide

Antigen-antibody complexes (i.e., immune complexes) can damage tissue by triggering Fc-receptor mediated inflammation, a process implicated in a variety of human diseases such as systemic lupus erythematosus, rheumatoid arthritis, and Sjoergen's syndrome. The effect of hCD47-Fc on immune complex (IC)-mediated inflammation was determined using in vitro assays that were developed to mimic IC-mediated cytokine induction in human dendritic cells by modifying previously described methods (see, e.g., Boruchov et al, J. Clin. Invest. 115:2914-23 (2005)). Dendritic cells (DC) were activated with IFN-γ and low dose of Toll like receptor (TLR) ligands (for example, FSL-1 or LPS) to resemble DC in inflamed tissue.

96 well plates were coated with 50 μl per well of 50 μg/ml anti-human Fc donkey IgG (Jackson ImmunoResearch Laboratories, West Grove, Pa.) in 0.1 M NaHCO₃/Na₂CO₃ buffer overnight. Then plates were washed twice with PBS and then incubated with either CD47-Fc or hFc-Stub at varying concentrations between 0.005 and 20 μg/ml (four-fold dilutions). After 2 h, plates were washed once with PBS before adding dendritic cells to the culture. Eight day-old human monocyte-derived DC (2×10⁴/well), prepared as described in Example 1, were added in the presence of IFN-γ (1000 U/ml). Alternatively, in control plates that were not coated with donkey anti-human Fc, DC were seeded into wells containing either soluble hCD47-Fc or hFc-Stub. After two hours, DC were stimulated with 0.1 ng/ml FSL-1 (a TLR-2 ligand) (InvivoGen, San Diego Calif.). Supernatants were collected after 18 hours and the concentration of TNF-α in the supernatants as described in Example 1.

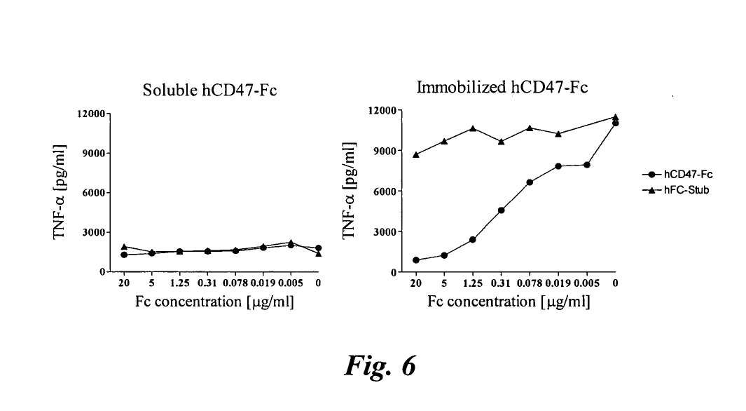

The results are presented in FIG. 6. The presence of donkey IgG (right side of FIG. 6) resulted in an approximately 6-fold increase in TNF-α production by human DC in response to the TLR2 ligand, FSL-1, when compared to DC in the absence of plate-bound IgG (left side of FIG. 6). hCD47-Fc inhibited the IgG-mediated increase in TNF-α production in a dose dependent manner when compared to the Fc-Stub control. hCD47-Fc had no effect on FSL-1-induced cytokine production in the absence of plate-bound IgG, demonstrating that the CD47 moiety of the hCD47-Fc fusion polypeptide inhibited the Fc-receptor mediated activation of DC.

### Example 3

### Inhibition of IgG-Induced Cytokine Production in Human Dendritic Cells by a Human CD47 Fusion Polypeptide Dimer

This Example describes the capability of a human CD47 extracellular domain fusion polypeptide dimer to inhibit IgG-mediated cytokine production in human dendritic cells.

A fusion polypeptide comprising the extracellular domain of human CD47 fused to a non-immunoglobulin moiety. As described herein, a fusion polypeptide dimer may form via the CD47 moieties of a fusion polypeptide, which are capable of forming an interchain disulfide bond. The non-immunoglobulin moiety fused to the CD47 moiety is referred to as a Hac moiety and comprises an hemagglutinin (HA) binding site, C-TAG (protein C-tag derived from the heavy chain of human protein C), and two streptavidin binding sites (2XSBP) (see, e.g., SEQ ID NO:34 sets forth the amino acid sequence of the HAC moiety, wherein the HA epitope is located at the amino terminal end of the Hac moiety fused to a C-TAG, which is fused to 2XSBP; SEQ ID NO:35 sets forth the nucleotide sequence encoding this Hac moiety). As described herein the extracellular domain of human CD47 may comprise the exemplary sequence set forth in SEQ ID NO:11 (with signal peptide sequence) or SEQ ID NO:1 (without the signal peptide sequence). The polypeptide was constructed according to molecular biology methods and protein expression methods routinely practiced by a person skilled in the art.

96 well plates were coated with 50 μl per well of 50 μg/ml mouse IgG (Jackson ImmunoResearch Laboratories, West Grove, Pa.) in 0.1 M NaHCO₃/Na₂CO₃ buffer overnight. Then plates were washed twice with PBS and then incubated with either hCD47-Hac or a control construct. The control constructs included a Gaussia luciferase fused to the same Hac moieity (gluc-Hac) or vCCI (also called p35), a soluble viral chemokine inhibitor from cowpox (p35-Hac). The hCd47-Hac and control construct were added to mouse IgG coated plates at varying concentrations between 0.001 and 0.8 g/ml (five-fold dilutions). After 2 h, plates were washed once with PBS before adding dendritic cells to the culture. Dendritic cells were obtained and prepared from two different donors. Eight day-old human monocyte-derived DC (2×10⁴/well), prepared as described in Example 1, were added in the presence of IFN-γ (1000 U/ml). Alternatively, in control plates that were not coated with mouse IgG, DC were seeded into wells containing either hCD47-Hac or the control construct. After two hours, DC were stimulated with 0.1 ng/ml FSL-1 (InvivoGen). Supernatants were collected after 18 hours and the concentration of TNF-α in the supernatants as described in Example 1.

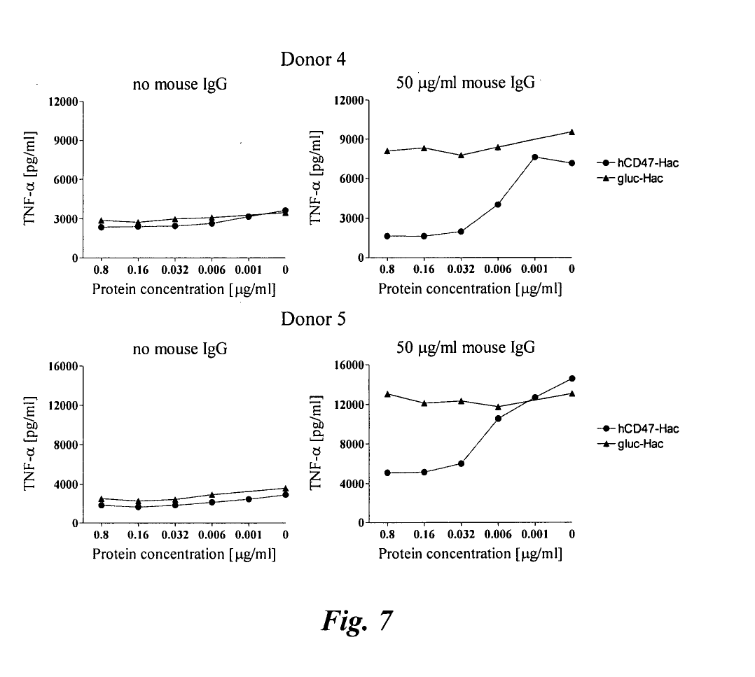

Results are presented in FIG. 7. Like hCD47-Fc, hCD47-Hac had no effect on TNF-α secretion by DC in response to FSL-1 in the absence of mouse IgG, but hCD47-Hac did inhibit immune complex-induced TNF-α production. The control construct gluc-Hac did not affect Fc-receptor-mediated cytokine production, confirming that the CD47 portion of the construct is blocking Fc-receptor mediated activation of DC.

### Example 4

### Inhibition of Fc-Mediated Cytokine Production in Bone Marrow Derived Murine Dendritic Cells by a Murine CD47-Fc Polypeptide Fusion Protein

Prior to performing animal studies in mouse animal models, experiments were performed to demonstrate that a murine CD47 extracellular domain-murine Fc fusion polypeptide interfered with (i.e., inhibited) IgG complex-Fc-receptor-induced inflammation.

A murine CD47-Fc construct containing the extracellular domain of murine CD47 and the Fc portion of murine IgG2a (mCD47-Fc) was expressed and purified. The murine CD47 extracellular domain was derived from the amino acid sequence encoded by the polynucleotide sequence set forth in GenBank Accession No. NM_{—}010581 (which provides the encoded amino acid sequence). Alternatively, the amino acid sequence of a murine CD47 set forth in GenBank Accession No. Q61735 (no version number provided) may be used.

To mimic immune-complex mediated inflammation, bone marrow derived DC (BMDC) and macrophages from BALB/c and C57BL/6 mice were stimulated with SAC-saturated with mIgG2a (IgG2a-SAC). BALB/c and C57BL/6 derived dendritic cells differ in cytokine production in response to SAC and SAC-IgG2a. In addition, investigators report that C57BL/6 mice express higher levels of Fc-receptors than BALB/c mice. Therefore, the effect of the mCD47-Fc construct on immune cells from two different strains of mice was examined.

Murine bone marrow derived DC were prepared according to the protocol of Lutz et al. (J. Immunol. Methods 223:77-92 (1999)). Cells were cultured in murine dendritic cell differentiation medium (IMDM (Invitrogen, Carlsbad, Calif.), 10% FBS (Hyclone, Logan, Utah), 50 μM 2-ME, 20 ng/ml murine GM-CSF (PeproTech) and 10 ng/ml murine IL-4 (PeproTech) for 9 days before use in cytokine assays. SAC-saturated with mIgG2a was prepared similarly to SAC saturated with human sera (see Example 1).

Nine day-old BMDC (2×10⁴ cells/96-well) were treated overnight in the presence of IFN-γ (1000 U/ml). Then varying concentrations (five-fold dilutions between 0.006 and 20 μg/ml) of either the mCD47-Fc polypeptide or a control molecule, in this instance, murine IgG2a were added to the cells, followed by stimulation with 0.01% IgG2a-SAC. Supernatants were collected from the stimulated cell cultures after 18 h and stored as described above until the concentrations of murine TNF-α and murine IL-12 were determined using ELISA kits according to the manufacturer's instructions.

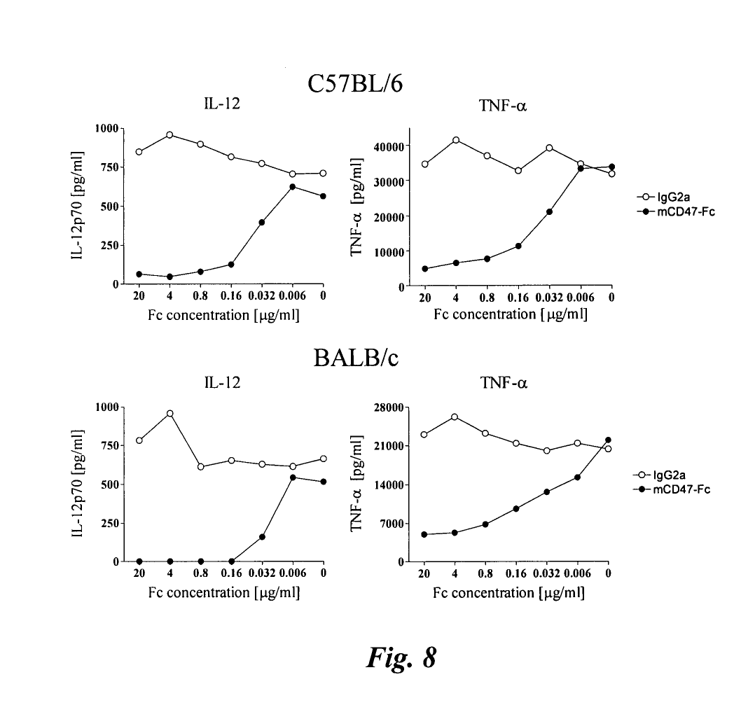

The results are presented in FIG. 8. Murine CD47-Fc inhibited IgG2a-SAC-induced IL-12p70 and TNF-α production in BMDC in a dose dependent manner, whereas the IgG2a control antibody did not reduce IgG2a-SAC-induced cytokine secretion.

### Example 5

### Effect of Fc-Mediated Cytokine Production in Bone Marrow Derived Murine Dendritic Cells by a Murine CD47-Fc Polypeptide Fusion Protein in Wild Type and FcRγ (−/−) Mice

To further demonstrate that mCD47-Fc blocks IgG/Fc-receptor mediated activation of DC, experiments were performed with DC from B57BL/6 FcRγ (−/−) and C57BL/6 wildtype controls. The B57BL/6 FcRγ (−/−) do not express the common γchain of activating Fc receptors. Thus, DC from FcRγ (−/−) mice do not produce inflammatory cytokines in response to complexed IgG.

BMDC from C57BL/6 FcRγ (−/−) and C57BL/6 wildtype controls were prepared as described in Example 4. Also as described in Example 4, nine day-old BMDC (2×10⁴ cells/96-well) were treated overnight in the presence of IFN-γ (1000 U/ml). Then varying concentrations (five-fold dilutions between 0.006 and 20 μg/ml) of either the mCD47-Fc polypeptide or the control molecule, murine IgG2a, were added to the cells, followed by stimulation with 0.01% IgG2a-SAC. Supernatants were collected from the stimulated cell cultures after 18 h and stored as described above until the concentrations of murine TNF-α in the supernatants were determined.

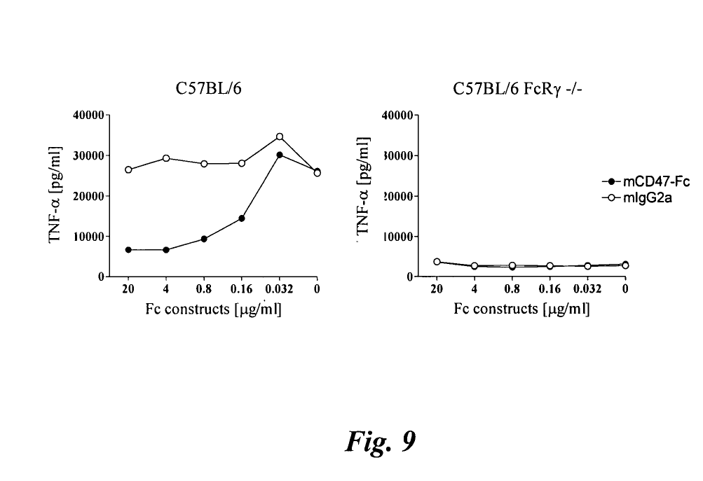

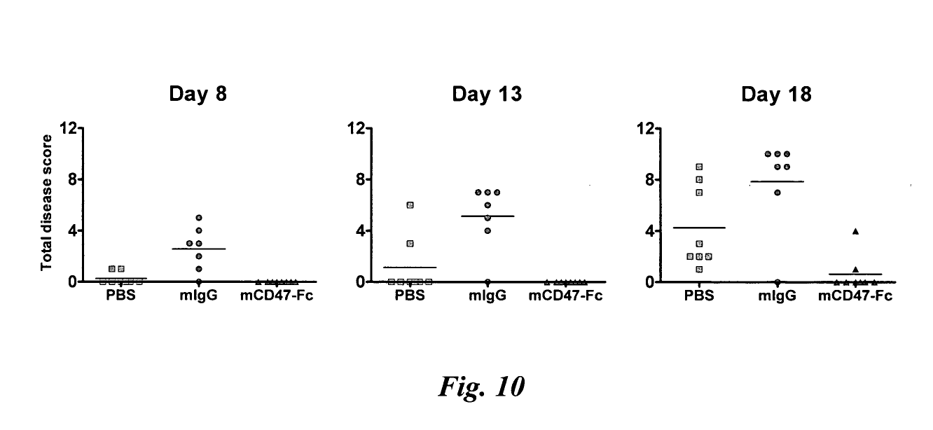

The results are presented in FIG. 9. IgG2a-SAC was a very potent stimulator of TNF-α in wildtype DC. Wildtype DC secreted approximately 80 times more TNF-α in response to IgG2a-SAC when compared to DC from FcRγ (−/−) mice. In DC from wildtype mice, mCD47-Fc inhibited IgG2a-SAC-induced cytokine production in a dose dependent manner (IC₅₀ below 0.16 μg/ml) when compared to the IgG2a control. Murine CD47-Fc did not affect TN F̅-α secretion in FcRγ (−/−) mice.

### Example 6

### Effect of a Murine CD47-Fc Polypeptide Fusion Protein on Development of Collagen Antibody-Induced Arthritis in a Murine Model

Because murine CD47-Fc inhibited Fc-receptor mediated cytokine production, mCD47-Fc was evaluated in the FcRγ dependent model of collagen antibody induced arthritis (CAIA) (Wallace et al., J. Immunol. 162:5547 (1999)) in DBA/1J mice (The Jackson Laboratory, Bar Harbor, Me.). Animal studies were performed according to Institutional Animal Care and Use Committee (IACUC) approved protocols. CAIA was induced in 8-week old male DBA/1J mice by intravenous injection (Day 0) of 4 mg of ArthritoMAB™ antibody (MD Biosciences Inc., St. Paul, Minn.) followed by intraperitoneal administration of an LPS (Escherichia coli 055:B5; Sigma BioSciences, St. Louis, Mo.) boost (50 μg) on Day 6 and again on Day 13. Prophylactic treatment of animals with mCD47-mFc (500 μg) or mIgG (500 μg) (from murine serum; Sigma Biosciences), or PBS began on day 0 (1 hour prior to ArthritoMAB™) and continued every other day until day 8. Clinical assessment of arthritis was determined by observers blinded to the treatment group. Mice paws were examined for disease severity and graded on a scale of 0 to 4 for each paw, according to changes in redness and swelling (0, normal; 1, mild swelling of a single area; 2, moderate swelling involving more than one area; 3, severe arthritis involving the entire paw; 4, severe arthritis resulting in ankylosis and loss of joint movement [deformity]). Each limb was graded, resulting in a maximal clinical severity score of 16 for each animal. The clinical severity score and number of paws affected were monitored daily during the entire study period.

Results are shown in FIG. 9. Prophylactic treatment of mice with mCD47-Fc reduced the incidence rates and total disease scores when compared to mice treated with PBS or murine IgG. On Day 18 post ArthritoMAB™ treatment, only 2 out of 8 mice treated with mCD47-Fc showed signs of disease, whereas all mice in the PBS group and 7 out of 8 mice in the mIgG treatment group showed signs of disease. Furthermore, the severity of inflammation was higher in the control groups with mean total disease scores of 4.2 in the PBS group and 7.9 in the mIgG treatment group compared to 0.6 in the mCD47-Fc treatment group.

From the foregoing, although specific embodiments of the invention have been described herein for purposes of illustration, various modifications may be made without deviating from the spirit and scope of the invention. Those skilled in the art will recognize, or be able to ascertain, using no more than routine experimentation, many equivalents to the specific embodiments of the invention described herein. Such equivalents are intended to be encompassed by the following claims.

1. A fusion polypeptide comprising an extracellular domain of CD47 fused to a human IgG1 Fc polypeptide, wherein the Fc polypeptide comprises a substitution or a deletion of a cysteine residue in the hinge region, wherein the substituted or deleted cysteine residue is the cysteine residue most proximal to the amino terminus of the hinge region of the Fc portion of a wildtype human IgG1 immunoglobulin, and wherein the Fc polypeptide further comprises a substitution or deletion of an aspartate residue immediately adjacent to the cysteine residue most proximal to the amino terminus of the hinge region of the Fc portion of a wildtype human IgG1 immunoglobulin, wherein the fusion polypeptide alters the immunoresponsiveness of an immune cell.

2.-3. (canceled)

4. The fusion polypeptide according to claim 1 wherein the fusion polypeptide comprises an amino acid sequence at least 95% identical to the amino acid sequence set forth in SEQ ID NO:2.

5. The fusion polypeptide according to claim 1 wherein the extracellular domain of human CD47 comprises an amino acid sequence at least 95% identical to the amino acid sequence set forth in SEQ ID NO:1 wherein the cysteine residues located at positions corresponding to positions 15, 23, and 96 of SEQ ID NO:1 are retained.

6. The fusion polypeptide according to claim 5 wherein the extracellular domain of human CD47 comprises the amino acid sequence set forth in SEQ ID NO:1.

7. The fusion polypeptide according to claim 1 wherein the extracellular domain of human CD47 comprises an amino acid sequence at least 95% identical to the sequence set forth in SEQ ID NO:11 wherein the cysteine residues located at positions corresponding to positions 33, 41, and 114 of SEQ ID NO:11 are retained.

8. The fusion polypeptide according to claim 1 wherein the extracellular domain of human CD47 comprises the amino acid sequence set forth in SEQ ID NO:11.

9. The fusion polypeptide according to claim 1 wherein the fusion polypeptide is capable of inhibiting immune complex-induced cytokine production in the immune cell.

10. The fusion polypeptide according to claim 1 wherein production of the cytokine IL-23 is inhibited.

11. The fusion polypeptide according to claim 1 wherein production of at least one cytokine selected from IL-23, IL-12, IL-6, and TNF-α is inhibited.

12. The fusion polypeptide according to claim 1 wherein the Fc polypeptide comprises at least one amino acid substitution to remove a glycosylation site.

13. The fusion polypeptide according to claim 12 wherein the Fc polypeptide is aglycosylated.

14. The fusion polypeptide according to claim 1 wherein the fusion polypeptide further comprises a polypeptide spacer between the Fc polypeptide and the extracellular domain of CD47.

15. The fusion polypeptide according to claim 14 wherein the polypeptide spacer comprises from 5 to 100 amino acid residues independently selected from glycine, asparagine, serine, threonine, and alanine.

16. The fusion polypeptide according to claim 15 wherein the polypeptide spacer comprises 5 to 20 amino acid residues.

17. The fusion polypeptide according to claim 15 wherein the polypeptide spacer comprises (Gly₄Ser)ₙ wherein n=1-12.

18. The fusion polypeptide according to claim 1 wherein the human IgG1 Fc polypeptide further comprises substitution of (a) at least one amino acid in the CH2 domain of the Fc polypeptide; (b) at least two amino acid residues in the CH2 domain of the Fc polypeptide or (c) at least three amino acid residues in the CH2 domain of the Fc polypeptide, such that the capability of the fusion polypeptide to bind to an IgG Fc receptor is reduced.

19. The fusion polypeptide according to claim 1, wherein the fusion polypeptide forms a dimer of two fusion polypeptide monomers, and wherein the dimer comprises a disulfide bond between each of the extracellular CD47 domain moieties of each of the two fusion polypeptide monomers.

20. The fusion polypeptide according to claim 19 wherein the disulfide bond between each of the extracellular domain moieties is formed between a cysteine residue of each extracellular CD47 domain moiety, which cysteine residue of each extracellular CD47 domain moiety is most proximal to the amino terminus.

21. The fusion polypeptide according to claim 20 wherein the CD47 extracellular domain variant retains the capability to bind at least one CD47 ligand selected from SIRP-α, SIRP-beta-2, thrombospondin-1, αᵥβ₃ integrin, and α₂β₁ integrin.

22. The fusion polypeptide according to claim 20 wherein the fusion polypeptide (a) competitively inhibits binding of at least one CD47 ligand to a CD47 polypeptide expressed on the cell surface of a cell and (b) competitively inhibits binding of a viral CD47-like polypeptide to at least one CD47 ligand.

23. The fusion polypeptide according to claim 22 wherein the at least one CD47 ligand is selected from SIRP-α, SIRP-beta-2, thrombospondin-1, αᵥβ₃ integrin, and α₂β₁ integrin.

24. The fusion polypeptide according to claim 23 wherein the fusion polypeptide competitively inhibits binding of at least one CD47 ligand to a cellular CD47 polypeptide by binding to the at least one CD47 ligand.

25. The fusion polypeptide according to claim 20 wherein altering immunoresponsiveness of the immune cell comprises at least one of (a) altering cell migration;
(b) inhibiting production of at least one cytokine selected from TNF-α, IL-12, IL-23, IFN-γ, GM-CSF, and IL-6; (c) inhibiting maturation of a dendritic cell; (d) impairing development of a naïve T cell into a Th1 effector cell; (e) inhibiting activation of the immune cell wherein the immune cell expresses SIRP-α on the cell surface; (f) inhibiting production of a chemokine; and (g) suppressing a proinflammatory response by the immune cell.

26. The fusion polypeptide according to claim 1 wherein the immune cell expresses SIRP-α on the cell surface, and wherein altering immunoresponsiveness of the immune cell comprises at least one of (a) inhibiting production of at least one cytokine in the immune cell wherein the cytokine is selected from TNF-α, IL-12, and IL-23; (b) inhibiting immune complex-induced cytokine production in the immune cell; and (c) inhibiting production of at least one chemokine, wherein the chemokine is MIP-1α.

27. The fusion polypeptide according to claim 26 wherein the immune cell is a dendritic cell.

28.-29. (canceled)

30. A polynucleotide encoding the fusion polypeptide according to claim 1.

31. A recombinant expression construct comprising the polynucleotide of claim 30 operatively linked to an expression control sequence.

32. A host cell transformed or transfected with the recombinant expression construct according to claim 31.

33. The host cell according to claim 32 wherein the host cell is a eukaryotic cell.

34. A composition comprising the fusion polypeptide according to claim 20 and a pharmaceutically suitable carrier.

35. A method of altering an immune response in a subject comprising administering to the subject the composition according to claim 34, thereby altering the immune response in the subject.

36. The method according to claim 35 wherein altering the immune response comprises suppressing the immune response.

37. A method of activating an immune cell comprising contacting the immune cell with the fusion polypeptide according to claim 20, under conditions and a time sufficient to permit the immune cell and the fusion polypeptide to interact, wherein a CD47 ligand is present on the cell surface of the immune cell, thereby activating the immune cell.

38. The method according to claim 37 wherein the CD47 ligand is selected from SIRP-α, SIRP-beta-2, thrombospondin-1, αᵥβ₃ integrin, and α₂β₁ integrin.

39. The method according to claim 38 wherein the CD47 ligand is SIRP-α.

40. The method according to claim 37 wherein activating the immune cell comprises inhibiting Fc-mediated cytokine production.

41. The method according to claim 40 wherein inhibiting Fc-mediated chemokine production comprises inhibiting immune complex-induced cytokine production.

42. The method according to claim 37 wherein activating the immune cell comprises inhibiting Fc-mediated chemokine production.

43. The method according to claim 37 wherein the immune cell is a dendritic cell, a monocyte, a granulocyte, or a bone marrow stem cell.

44. A method of inhibiting immune complex-induced cytokine production in an immune cell comprising contacting an immune cell with the fusion polypeptide according to claim 20 under conditions and for a time sufficient to inhibit immune complex-induced cytokine production in the immune cell.

45. The method according to claim 36 wherein the immune cell expresses a CD47 ligand on the cell surface.

46. The method according to claim 45 wherein the CD47 ligand is selected from SIRP-α, SIRP-beta-2, thrombospondin-1, αᵥβ₃ integrin, and α₂β₁ integrin.

47. The method according to claim 44 wherein the immune cell is a dendritic cell, a monocyte, a granulocyte, or a bone marrow stem cell.

48. The method according to claim 44 wherein the immune cell is a dendritic cell and the CD47 ligand is SIRP-α.

49. The method according to claim 44 wherein production of at least one cytokine selected from IL-23, IL-12, IL-6, and TNF-α is inhibited.

50. The method according to claim 44 wherein the fusion polypeptide inhibits binding of the immune complex to the immune cell.

51. A method of treating an immunological disease or disorder in a subject who has or who is at risk of developing the immunological disease or disorder, said method comprising administering to the subject the composition according to claim 34.

52.-54. (canceled)

55. The method according to claim 52 wherein the immunological disease or disorder is caused by or exacerbated by binding of an immune complex to an immune cell.

56. The method according to claim 55 wherein the immune cell is a dendritic cell.

57. A method of manufacture for producing the fusion polypeptide according to claim 20.

58. An isolated antibody, or antigen-binding fragment thereof, (a) that specifically binds to CD47 and (b) that competitively inhibits binding of a CD47 ligand (i) to CD47 and (ii) to a viral CD47-like polypeptide.

59. The antibody, or antigen-binding fragment thereof, according to claim 58 wherein the viral CD47-like polypeptide is a poxvirus CD47-like polypeptide.

60.-61. (canceled)

62. The antibody, or antigen-binding fragment thereof, according to claim 58 wherein the antibody, or antigen-binding fragment thereof, inhibits Fc-mediated cytokine production or chemokine production by an immune cell.

63. The antibody, or antigen-binding fragment thereof, according to claim 62, wherein inhibiting Fc-mediated cytokine production or chemokine production comprises inhibiting immune complex-induced cytokine production or chemokine production by an immune cell.

64.-70. (canceled)

71. A host cell that expresses the antibody of claim 58.

72. The host cell of claim 71 that is a hybridoma cell.

73.-75. (canceled)

76. An agent that specifically binds to CD47 and that inhibits binding of a viral CD47-like polypeptide to at least one CD47 ligand.

77.-80. (canceled)

81. A composition comprising the antibody, or antigen-binding fragment thereof, according to claim 58 and a pharmaceutically suitable carrier.

82. (canceled)

83. A method for identifying an agent that alters immunoresponsiveness of an immune cell comprising:
(a) contacting (i) a candidate agent; (ii) a viral CD47-like polypeptide; and (iii) a CD47 ligand, under conditions and for a time sufficient to permit interaction between the CD47 ligand and the viral CD47-like polypeptide;
(b) determining a level of binding of the viral CD47-like polypeptide to the CD47 ligand in the presence of the candidate agent and comparing a level of binding of the viral CD47-like polypeptide to the CD47 ligand in the absence of the candidate agent, wherein a decrease in the level of binding of the viral CD47-like polypeptide to the CD47 ligand in the presence of the candidate agent indicates that the candidate agent inhibits binding of the viral CD47-like polypeptide to the CD47 ligand;
(c) contacting (i) the candidate agent; (ii) a CD47 ligand; and (iii) an immune cell that expresses CD47, under conditions and for a time sufficient to permit interaction between a CD47 ligand and CD47; and
(d) determining a level of binding of the CD47 ligand to the immune cell in the presence of the candidate agent and comparing a level of binding of the CD47 ligand to the immune cell in the absence of the candidate agent, wherein a decrease in the level of binding of the CD47 ligand to the immune cell in the presence of the candidate agent indicates that the candidate agent alters immunoresponsiveness of the immune cell.

84.-86. (canceled)

87. A method of altering an immune response in a subject comprising administering to the subject the composition according to claim 81, thereby altering the immune response of the subject.

88. (canceled)

89. A method of altering an immune response in a subject comprising administering to the subject the composition according to claim 82, thereby altering the immune response of the subject.

90. (canceled)

91. A method for treating an immunological disease or disorder in a subject comprising administering to the subject the composition according to claim 81.

92.-94. (canceled)

95. A method for treating an immunological disease or disorder in a subject comprising administering to the subject a composition that comprises (a) a pharmaceutically suitable carrier; and either (i) an agent that specifically binds to CD47 and that impairs binding of a viral CD47-like polypeptide to a CD47 ligand or (ii) an agent that specifically binds to a CD47 ligand and specifically impairs binding of a viral CD47-like polypeptide to the CD47 ligand.

96.-99. (canceled)

100. A method of treating a disease or disorder associated with alteration of at least one of cell migration, cell proliferation, and cell differentiation in a subject, wherein the method comprises administering to the subject a pharmaceutically suitable carrier and either (a) an agent that specifically binds to CD47 and that impairs binding of a viral CD47-like polypeptide to a CD47 ligand or (b) an agent that specifically binds to a CD47 ligand and specifically impairs binding of a viral CD47-like polypeptide to the CD47 ligand.

101. The method according to claim 100 wherein the viral CD47-like polypeptide is a poxvirus CD47-like polypeptide.

102.-110. (canceled)

111. A method of activating an immune cell comprising contacting the immune cell with either (a) an agent that specifically binds to CD47 and that impairs binding of a viral CD47-like polypeptide to a CD47 ligand, or (b) an agent that specifically binds to a CD47 ligand and that impairs binding of a viral CD47-like polypeptide to a CD47 ligand under conditions and a time sufficient to permit the immune cell and the agent to interact, wherein the CD47 ligand is present on the cell surface of the immune cell, thereby activating the immune cell.

112.-119. (canceled)

120. A method of manufacture for producing an agent that suppresses immunoresponsiveness of an immune cell, comprising:
(a) identifying an agent that alters immunoresponsiveness of an immune cell, wherein the step of identifying comprises:
(I) contacting (i) a candidate agent; (ii) a viral CD47-like polypeptide; and
(iii) a CD47 ligand, under conditions and for a time sufficient to permit interaction between the CD47 ligand and the viral CD47-like polypeptide; and
(II) determining a level of binding of the viral CD47-like polypeptide to the CD47 ligand in the presence of the candidate agent and comparing a level of binding of the viral CD47-like polypeptide to the CD47 ligand in the absence of the candidate agent, wherein a decrease in the level of binding of the viral CD47-like polypeptide to the CD47 ligand in the presence of the candidate agent indicates that the candidate agent inhibits binding of the viral CD47-like polypeptide to the CD47 ligand;
(III) contacting (i) the candidate agent; (ii) a CD47 ligand; and (2) an immune cell that expresses CD47, under conditions and for a time sufficient to permit interaction between the CD47 ligand and CD47; and
(IV) determining a level of binding of the CD47 ligand to the immune cell in the presence of the candidate agent and comparing a level of binding of the CD47 ligand to the immune cell in the absence of the candidate agent, wherein a decrease in the level of binding of the CD47 ligand to the immune cell in the presence of the candidate agent indicates that the candidate agent alters immunoresponsiveness of the immune cell; and
(b) producing the agent identified in step (a).

121.-126. (canceled)

127. A method of manufacture for producing the antibody, or antigen-binding fragment thereof, according to claim 58.

128. A fusion polypeptide comprising an extracellular domain of a viral CD47 polypeptide fused to an Fc polypeptide.

129.-131. (canceled)
